SEQUENCE LISTING (17 sequences)

SEQ ID NO: 1 - protein, 88 aa, Homo sapiens
KSGVLFLLGIILLVLIGVQGTPVVRKGRCSCISTNQGTIHLQSLKDLKQFAPSPSCEKIEIIATLKNGVQTCLNPDSADVKELIKKWE

SEQ ID NO: 2 - protein, 88 aa, Mus musculus
KSAVLFLLGIIFLEQCGVRGTLVIRNARCSCISTSRGTIHYKSLKDLKQFAPSPNCNKTEIIATLKNGDQTCLDPDSANVKKLMKEWE

SEQ ID NO: 3 - protein, 88 aa, Artificial Sequence
  Modified human CXCL9 chemokin
KSXVLFLLGIIXLXXXGVXGTXVXRXXRCSCISTXXGTIHXXSLKDLKQFAPSPXCXKXEIIATLKNGXQTCLXPDSAXVKXLXKXWE

SEQ ID NO: 4 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 5 - protein, 98 aa, Mus musculus
MNPSAAVIFCLILLGLSGTQGIPLARTVRCNCIHIDDGPVRMRAIGKLEIIPASLSCPRVEIIATMKKNDEQRCLNPESKTIKNLMKAFSQKRSKRAP

SEQ ID NO: 6 - protein, 98 aa, Artificial Sequence
  Modified human CXCL10 chemokin
MNXXAXXIXCLIXLXLSGXQGXPLXRTVRCXCIXIXXXPVXXRXXXKLEIIPASXXCPRVEIIATMKKXXEXRCLNPESKXIKNLXKAXSXXRSKRXP

SEQ ID NO: 7 - protein, 75 aa, Homo sapiens
QGFPMFKRGRCLCIGPGVKAVKVADIEKASIMYPSNNCDKIEVIITLKENKGQRCLNPKSKQARLIIKKVERKNF

SEQ ID NO: 8 - protein, 75 aa, Mus musculus
QGFLMFKQGRCLCIGPGMKAVKMAEIEKASVIYPSNGCDKVEVIVTMKAHKRQRCLDPRSKQARLIMQAIEKKNF

SEQ ID NO: 9 - protein, 75 aa, Artificial Sequence
  Modified human CXCL11 chemokin
QGFXMFKXGRCLCIGPGXKAVKXAXIEKASXXYPSNXCDKXEVIXTXKXXKXQRCLXPXSKQARLIXXXXEXKNF

SEQ ID NO: 10 - protein, 297 aa, Bacillus anthracis
MKAKTLSRHLREGVKNLSRNGWMTFASVSAVTVTLLLVGVFLTAIMNMNHFATKVEQDVEIRVHIDPAAKEADQKKLEDDMSKIAKVESIKYSSKEEELKRLIKSLGDSGKTFELFEQDNPLKNVFVVKAKEPTDTATIAKKIEKMQFVSNVQYGKGQVERLFDTVKTGRNIGIVLIAGLLFTAMFLISNTIKITIYARSTEIEIMKLVGATNWFIRWPFLLEGLFLGVLGSIIPIGLILVTYNSLQGMFNEKLGGTIFELLPYSPFVFQLAGLLVLIGALIGMWGSVMSIRRFLKV

SEQ ID NO: 11 - DNA, 894 bp, Bacillus anthracis
atgaaggctaagacccttagtcgacatttgcgagaaggtgtgaaaaatctatcccgtaacggatggatgacgtttgcttctgttagtgcagtaacagttacactattacttgtaggtgtctttttaacagcgattatgaatatgaaccattttgcgacgaaagtagagcaagatgttgagattcgtgtacacattgatccagcagcaaaagaagctgatcaaaagaaattagaagatgatatgagtaagattgcaaaagtagaatctattaaatattcttctaaagaagaagagttaaaacgtttaattaaaagcttaggcgatagcggaaagacgtttgagttatttgaacaagataacccactgaaaaacgtgttcgttgtaaaagcgaaagaaccaacagatacagcaacaattgcgaaaaagattgaaaaaatgcagtttgtaagtaatgttcagtacggaaaagggcaagttgaacgattatttgatactgtaaaaactggtcgtaacattggtattgtgttaattgctggtcttttattcacagcgatgttcttaatctctaacacaattaaaattacaatttatgctcgtagtacagaaatcgaaattatgaaacttgtaggtgcaacaaactggtttattcgttggccgttcttgttagagggattattcctaggagtattaggatcaattattccaattggcttaattcttgttacgtataattcactacaaggtatgtttaacgaaaaacttggcggaacaattttcgaacttctaccatatagtccgttcgtattccaattagctggtttactagtattaattggggctttaatcggtatgtggggaagcgtaatgtcaattcgtcgtttcttaaaagtataa

SEQ ID NO: 12 - protein, 88 aa, Artificial Sequence; identical to SEQ ID NO: 3
  Modified human CXCL9 chemokin

SEQ ID NO: 13 - protein, 98 aa, Artificial Sequence; identical to SEQ ID NO: 6
  Modified human CXCL10 chemokin

SEQ ID NO: 14 - protein, 75 aa, Artificial Sequence; identical to SEQ ID NO: 9
  Modified human CXCL11 chemokin

SEQ ID NO: 15 - protein, 88 aa, Artificial Sequence
  Modified human CXCL9 chemokin
KSGVLFLLGIILLVLIGVXGTPVXRKGRCSCISTXXGTIHLXSLKDLKQFAPSPXCEKIEIIATLKNGVQTCLXPDSAXVKXLXKXWE

SEQ ID NO: 16 - protein, 98 aa, Artificial Sequence
  Modified human CXCL10 chemokin
MNQXAIXICCLIFLTLSGIQGXPLXRTVRCTCISISXQPVNPRXXEKLEIIPASQFCPRVEIIATMKKKGEXRCLNPESKAIKNLXKAVSXXRSKRXP

SEQ ID NO: 17 - protein, 75 aa, Artificial Sequence
  Modified human CXCL11 chemokin
QGFPMFKXGRCLCIGPGXKAVKXAXIEKASXXYPSNNCDKXEVIXTXKEXKGQRCLXPXSKQARLIXXKXEXKNF

(19) United States
(12) Patent Application Publication
(10) Pub. No.: US 2012/0321687 A1
(43) Pub. Date: Dec. 20, 2012

(54) COMPOSITIONS AND METHODS FOR USING AND IDENTIFYING ANTIMICROBIAL AGENTS

(75) Inventors: Molly A. Hughes, Charlottesville, VA (US); Robert M. Strieter, Charlottesville, VA (US)

(73) Assignee: UNIVERSITY OF VIRGINIA PATENT FOUNDATION, Charlottesville, VA (US)

(21) Appl. No.: 13/579,581

(22) PCT Filed: Feb. 18, 2011
(86) PCT No.: PCT/US11/25473

§ 371 Date: Aug. 17, 2012

Related U.S. Application Data
(60) Provisional application No. 61/306,106, filed on Feb. 19, 2010.
(60) Provisional application No. 61/437,371, filed on Jan. 28, 2011.

(51) Int. Cl. A01N 37/46; A61K 39/07; A01N 25/34; A61K 38/19; A01P 1/00; A61P 31/04; A61K 9/00; C07K 14/32

(52) U.S. Cl. 424/405; 424/400; 424/190.1; 530/350; 514/2.3; 514/2.4

(57) ABSTRACT

The present invention provides proteins with antimicrobial activity, and methods for treating subjects by administering the proteins. In particular, the invention provides methods for treating and/or preventing microbial diseases and infections. The present invention further provides the target for these antimicrobial agents, as well as assays for identifying regulators of the target.

### CROSS REFERENCE TO RELATED APPLICATIONS

This application claims priority under 35 USC §119(e) to U.S. provisional application Ser. Nos. 61/306,106, filed Feb. 19, 2010 and 61/437,371, filed Jan. 28, 2011, the disclosures of which are incorporated herein by reference.

### INCORPORATION OF SEQUENCE LISTING

The sequence listing with file name SEQList_ST25.txt, created on Feb. 18, 2011 (32.9 KB) is expressly incorporated by reference in its entirety.

### BACKGROUND

Pathogenic microbes are increasingly becoming resistant to established antibiotic drugs. Only three new structural classes of antibiotics have been introduced into medical practice in the past 40 years and certain pathogenic bacteria have become resistant to all these classes. Moreover, all antimicrobial drugs on the market have some relative degree of host toxicity that is concentration dependent.

B. anthracis is a Gram-positive, spore-forming bacterium that is the causative agent of anthrax. There are three clinical forms of anthrax that reflect the route by which the bacterial spores are introduced in the host: cutaneous, gastrointestinal, and inhalational. Inhalational anthrax is a disease that has been described as a biphasic clinical illness characterized by a 1- to 4-day initial phase of malaise, fatigue, fever, myalgias, and nonproductive cough. The initial phase is then followed by a rapidly fulminant phase of respiratory distress, cyanosis, and diaphoresis. Death typically follows the onset of the fulminant phase in 1 to 2 days. Inhalational anthrax typically causes severe necrotizing pneumonia, mediastinal invasive disease with resultant massive hemorrhagic mediastinitis and lymphadenitis, and dissemination to other organs, including the central nervous system, gastrointestinal tract, lymph nodes, and vascular system. Since the initial phase of illness can be confused with a non-specific viral respiratory infection, a diagnosis of anthrax is often not entertained. Mortality is high (>85%) if the diagnosis is delayed. In fact, even in the 2001 human anthrax cases mortality was still high (45%) in spite of early recognition of inhalational anthrax infection in the index case, early awareness of the nefarious distribution of anthrax spores through the United States postal system, and alerts raised amongst health care providers, leading to empirical pre-emptive antibiotic intervention.

Because B. anthracis exists in two distinct forms (spores and vegetative cells, i.e., bacilli) and causes human pulmonary infection with disseminated disease, studies on B. anthracis have a broader applicability for understanding host-pathogen interactions and host response to bacterial pulmonary pathogens. The spores of B. anthracis are the infectious form of the organism and are responsible for initiating all forms of clinical anthrax. Spores are extremely hardy and can withstand extremes of heat, mechanical disruption, ultraviolet irradiation, and lytic enzymes. Spores are comprised of multiple protective layers that consist, from the inside to the outside, of a nucleic acid core surrounded by an inner spore membrane, cortex, outer spore membrane, spore coat, and exosporium.

The dominant model of inhalational anthrax involves the uptake of spores by alveolar macrophages or other phagocytic cells with subsequent transport by the phagocytic cells to the mediastinal lymph nodes. Spore germination and outgrowth of vegetative bacilli occur primarily in the host cell cytosol, and the organisms eventually escape from the host cell and disseminate throughout the host. All known virulence factors of B. anthracis are produced by the vegetative bacilli. The virulence factors include two bipartite toxins (lethal toxin and edema toxin) and a poly-gamma-D-glutamic acid capsule.

The B. anthracis Ames strain possesses two plasmids that encode the genes for the synthesis of the toxins and capsule (plasmids pXO1 and pXO2, respectively). Sterne strain possesses pXO1 but not pXO2; thus, Sterne strain is a toxigenic, unencapsulated B. anthracis strain. The majority of our data has been generated using Sterne strain, although select experiments have also been performed with Ames strain to confirm that Sterne strain is a suitable model organism for our studies.

Much work has been devoted in the anthrax field towards understanding critical host factors in anthrax infection. In vitro experiments and in vivo work in mice have revealed the genetic locus for Nalp1b appears to be a determinant of susceptibility, and defects in Nalp1b in mouse macrophages lead to decreased in vitro release of the pro-inflammatory cytokine, IL-1β. Furthermore, there are distinct differences in mouse strain susceptibility to anthrax. For example, the CS-deficient A/J mice are highly susceptible to inhalational (or subcutaneous) infection with B. anthracis Sterne strain. In contrast, the majority of mouse strains tested, including C57BL/6 mice, are resistant to inhalational anthrax infection with B. anthracis Sterne strain. However, C57BL/6 mice are susceptible to B. anthracis administered by other routes of inoculation (e.g., subcutaneous injection), which would suggest that important host defense factors are present or generated in the lungs that are otherwise bypassed when the organisms are introduced by another route.

In terms of the human host, susceptibility factors have not been identified although age and diminished host immune response are likely candidates, based on the observation that mortality from inhalational anthrax in the 1972 accidental release of spores in the city of Sverdlovsk in the former Soviet Union, as well as the 2001 anthrax cases that occurred from intentional delivery of spores through the U.S. postal system, occurred solely in adults and primarily, adults over the age of 35.

In a follow-up study of persons exposed to B. anthracis spores in the U.S. Capitol building in 2001, adults in the “definite exposure” group (based on exposure zone and positive nasopharyngeal swab cultures for B. anthracis), but who did not subsequently develop or succumb to clinical anthrax, had markers indicative of a cell-mediated immune response with elevated levels of TNF-α, IL-1β, IL-6, and CXCL9. Although it remains unclear which host factors play a role in susceptibility in humans, an early cell-mediated host immune response is likely a critical factor in this pulmonary infection that has a rapidly fatal course if untreated, especially given that there is no time for the host to mount an antibody-mediated immune response. An unfortunate twist is that lethal toxin and to a lesser extent, edema toxin are now recognized to play an important role in altering the host immune response—this is an evolving story of toxin-mediated host immune suppression, and the effects appear to be quite complex, depending on whether one is studying toxin effects in vitro or in vivo. It appears, however, that B. anthracis may be particularly skillful at surviving in the host, likely through toxin suppression of the immune response. Additionally, bacterial toxins are not affected by antibiotics and as such, once the toxins are being produced, the window of opportunity rapidly closes for containing and fighting this infection with standard treatment modalities (i.e., antibiotics).

Another critically important issue is that B. anthracis, like many other organisms, can acquire or develop antibiotic resistance such that antibiotic choices will become limited. Thus, critically important factors that can facilitate successful recovery of the host from this infection include a combination of appropriate therapeutic intervention plus an effective host immune response.

Chemokines are chemotactic cytokines that are important regulators of leukocyte-mediated inflammation and immunity in response to a variety of diseases and infectious processes in the host. Chemokines are a superfamily of homologous 8-10 kDa heparin-binding proteins, originally identified for their role in mediating leukocyte recruitment.

The four major families of chemokine ligands are classified on the basis of a conserved amino acid sequence at their amino terminus, and are designated CXC, CC, C, and CX3C sub-families (where “X” is a nonconserved amino acid residue; reviewed in references 76, 78).

The interferon-inducible (ELR−) CXC chemokines are one of the largest families of chemokines, and each member of this group contains four cysteine residues. Most chemokines are small proteins (8-10 kDa in size), have a net positive charge at neutral pH, and share considerable amino acid sequence homology. Structurally, the defining feature of the CXC chemokine family is a motif of four conserved cysteine residues, the first two of which are separated by a non-conserved amino acid, thus constituting the Cys-X-Cys or ‘CXC’ motif This family is further subdivided on the basis of the presence or absence of another three amino acid sequence, glutamic acid-leucine-arginine (the ‘ELR’ motif), immediately proximal to the CXC sequence (see references 75, 119). The ELR− positive (ELR+) CXC chemokines, which include IL-8/CXCL8, are potent neutrophil chemoattractants and promote angiogenesis. Among the ELR− negative (ELR−) CXC chemokines, CXCL9, CXCL10 and CXCL11, are potently induced by both type 1 and type 2 interferons (IFN-α/β and IFN-γ, respectively). These Interferon-inducible (ELR−) CXC chemokines are generated by a variety of cell types (including monocytes, macrophages, lymphocytes, and epithelial cells), and are extremely potent chemoattractants for recruiting mononuclear leukocytes, including activated Th1 CD4 T cells, natural killer (NK) cells, NKT cells, and dendritic cells to sites of inflammation and inhibiting angiogenesis.

The chemokine receptors are a family of related receptors that are expressed on the surface of all leukocytes. The shared receptor for CXCL9, CXCL10, and CXCL11 is CXCR3 (see references 69, 72, 92, 97, 111). Through their interaction with CXCR3, the ligands CXCL9, CXCL10 and CXCL11 are the major recruiters of specific leukocytes, including CD4 T cells, NK cells, and myeloid dendritic cells. Importantly, this chemokine ligand-receptor system is at the core of a positive feedback loop escalating Th1 immunity, whereby cytokines such as interleukin (IL)-12 and IL-18 (released by myeloid accessory cells) activate local NK cells to produce IFN-γ, which then induces generation of CXCL9, CXCL10, and CXCL11, which then recruits CXCR3-expressing cells that act as a further source of IFN-γ, which then induces further production of CXCL9, CXCL10, and CXCL11. Consistent with the importance of these interferon-inducible (ELR−) CXC chemokines in promoting Thl-mediated immunity, CXCR3 and its ligands have been documented to play a critical role in host defense against many micro-organisms, including viruses, Mycobacterium tuberculosis, other bacteria, and protozoa.

Independent of their role in CXCR3-dependent leukocyte recruitment, CXCL9, CXCL10, and CXCL11 have recently been found to display direct antimicrobial properties that resemble those of defensins (see references 33, 40). These antimicrobial effects were first demonstrated in 2001 against Escherichia coli and Listeria monocytogenes. Subsequently, an increasing number of chemokines have been shown to have antimicrobial activity against various strains of bacteria and fungi, including E. coli, S. aureus, Candida albicans, and Cryptococcus neoformans (see references 112, 114).

There is a long felt need in the art for new compositions and methods useful as antimicrobial agents, as well as targets for antimicrobial agents. The present invention satisfies these needs.

### SUMMARY OF THE INVENTION

The present disclosure provides methods for treating and/or preventing microbial diseases. The invention also provides methods for treating and/or preventing microbial infections. In accordance with one embodiment compositions comprising interferon-inducible (ELR−) CXC chemokines, including for example chemokines CXCL9, CXCL10 and CXCL11, can be used to neutralize actively growing, as well as stationary phase, pathogenic bacteria. Furthermore, the chemokine compositions of the present invention have been discovered to be surprisingly effective in neutralizing the spores of pathogenic bacteria, including spores of Bacillus anthracis. The compositions disclosed herein can be used as a therapeutic intervention and innovative approach for treating pulmonary and gastrointestinal bacterial pathogens, especially at a time when it is becoming increasingly clear that expanding antibiotic resistance in bacterial pathogens is moving the medical field into a post-antibiotic era.

In some embodiments, the methods of the invention comprise administering to a subject a therapeutically effective amount of at least one compound of the invention. In one aspect, the compound is a peptide, or a fragment, homolog, or modification thereof. In one aspect, an isolated nucleic acid comprising a nucleic acid sequence encoding a peptide of the invention is administered.

The present invention encompasses the theory disclosed herein that, inter alia, interferon-inducible (ELR−) CXC chemokines exhibit antimicrobial activity. In one aspect, the microbes are bacteria. In another aspect, the bacteria include Gram-positive and Gram-negative bacteria.

It is also disclosed herein that FtsX is the putative bacterial target for interferon-inducible (ELR−) CXC chemokines in B. anthracis. The present invention therefore encompasses targeting FtsX either directly or indirectly for use as an antimicrobial agent or target of an antimicrobial agent. The present invention further provides compositions and methods useful for identifying regulators of FtsX, and therefore, identifying antimicrobial agents. In one aspect, the present invention provides compositions, methods, and assays utilizing FtsX to identify compounds that regulate FtsX function or levels or downstream activity. In one aspect, the regulation is inhibition. In one aspect, compounds identified in these assays exhibit anti-microbial activity as described herein. The types of compounds useful in the invention include, but are not limited to, proteins and peptides, as well as active fragments and homologs thereof, drugs, and peptide mimetics. In one aspect, the active fragments, homologs, and mimetics are fragments, homologs, and mimetics or agonists of the chemokines described herein.

It is disclosed herein that FtsX is a target of interferon-inducible (ELR−) chemokines and that these chemokines have antimicrobial activity against bacteria expressing FtsX. Therefore, the present invention encompasses the use of isolated FtsX as a vaccine or therapeutic immunogenic agent useful for preventing or treating infections or diseases involving FtsX-expressing microbes. In one aspect, an isolated nucleic acid comprising a sequence encoding FtsX or a fragment or homolog thereof can be administered to a subject in need thereof In another aspect, an immunogenic amount of an isolated FtsX protein, or a fragment of homolog thereof can be administered to a subject in need thereof.

Further embodiments of the invention include therapeutic kits that comprise, in suitable container means, a pharmaceutical formulation of at least one antimicrobial peptide of the invention. Some embodiments provide kits comprising a pharmaceutical formulation comprising at least one peptide of the invention and a pharmaceutical formulation of at least one antimicrobial agent or antibiotic. The antimicrobial peptide and antimicrobial agent or antibiotic may be contained within a single container means, or a plurality of distinct containers may be employed.

Various aspects and embodiments of the invention are described in further detail below.

### BRIEF DESCRIPTION OF THE DRAWINGS

FIG. 1 is a schematic drawing of the three dimensional structure of an interferon-inducible (ELR−) CXC chemokine (figure taken from Frederick, M. J., and G. L. Clayman (2001) Expert Rev. Mol. Med. (01)00330-1a.pdf (short code: txt001mfh); 18 Jul. 2001).

FIGS. 2A & 2B. EM images of control and CXCL10 treated Sterne strain vegetative bacilli. Vegetative cells were incubated with buffer (FIG. 2A) or CXCL10 (FIG. 2B; 48 μg/ml) for 30 min, fixed, permeabilized, processed for CXCL10 immunogold labeling with silver enhancement, and imaged via EM at 30,000× magnification. CXCL10 localization appears primarily along the cell membrane (FIG. 2B, black arrows) of bacilli, which have also begun to lose structural integrity even at this early time point. Scale bar represents 0.5 μm.

FIG. 3A. Human CXCL10 has direct effects against B. anthracis Ames strain encapsulated bacilli. Under BSL-3 conditions, encapsulated Ames bacilli were incubated with buffer alone or CXCL10 (48 μg/ml) for 6 hr. Aliquots of samples were then plated onto BHI plates. CFU determination was performed after overnight incubation. ***p-value<0.001, compared to untreated (buffer) control sample (FIG. 3A). Initial bacilli inoculum is indicated with a dashed line. The presence of capsule was verified for each starting sample using India ink stain and visualization under light microscopy. Data represent two separate experiments performed in triplicate each time.

FIG. 3B. Human interferon-inducible (ELR−) CXC chemokines, CXCL9, CXCL10, and CXCL11 exhibit antimicrobial activity against B. anthracis Sterne strain. FIG. 3B is a bar graph of data demonstrating that the recombinant human interferon-inducible (ELR−) CXC chemokines, CXCL9, CXCL10, and CXCL11 (48 μg/ml) incubated with B. anthracis Sterne strain bacilli for 6 hr have activity as anti-microbial agents; in contrast, two CC chemokines (CCL2 and CCL5), which have similar charge and molecular mass to those of the interferon-inducible (ELR−) CXC chemokines, do not exhibit antimicrobial activity against B. anthracis. ***p-value<0.001, dashed line represents initial inoculum concentration, (n.d.)=none detected, n=3 experiments. Human CXCL10 was more effective than human CXCL9, which was more effective than human CXCL11.

FIG. 3C. Murine interferon-inducible (ELR−) CXC chemokines, CXCL9, CXCL10, and CXCL11 exhibit antimicrobial activity against B. anthracis Sterne strain. Susceptibility of B. anthracis Sterne strain bacilli to recombinant murine interferon-inducible (ELR−) CXC chemokines, CXCL9, CXCL10, and CXCL11 (48 ug/ml for 6 hr) was tested using an Alamar Blue assay (see FIG. 3C). Murine CXCL9 was more effective than murine CXCL10, which was approximately as effective as murine CXCL11. **p-value<0.01, ***p-value<0.001, (n.d.)=none detected, n=3 experiments.

FIGS. 4A & 4B. Isolation and confirmation of CXCL10-resistant isolates from B. anthracis transposon mutagenesis library screen. Using a mariner transposon mutagenesis library of B. anthracis Sterne strain, a pool of vegetative cells grown from the library (>50,000 CFU's, representing ˜10× genome coverage) was incubated with 48 μg/ml CXCL10 or buffer only (untreated) for 1 hr at 37° C. Vegetative cells were plated onto BHI plates+erythromycin (selection marker for library). For untreated cells, a lawn of colonies was obtained, but for a CXCL10-treated library, 13 colonies were obtained from one screen (FIG. 4A), and a total of 18 colonies were obtained from two separate screens. Each of the 18 isolates (TNX1-18) was tested for resistance to CXCL10 using an Alamar Blue assay (FIG. 4B). **p-value<0.01, ***p-value<0.001 compared to the B. anthracis Sterne strain 7702 (wildtype strain, designated “7702”) and compared to CXCL10-treated library (designated “Library”); n.d.=not detectable.

FIGS. 5A & 5B. Schematic of prototypical ABC transporters that function as importers or exporters. The prototype in Gram-negative bacteria is highlighted in the schematic drawing of FIG. 5A and the prototype in Gram-positive bacteria is highlighted in the schematic drawing of FIG. 5B. The typical components of the ABC transporter consist of a substrate binding protein (SBP), a membrane-spanning domain (MSD) as a heterodimer, and an ATPase or nucleotide binding protein (NBP). Figure taken from Braibant M., P. Gilot, and J. Content (2000) FEMS Microbiol. Rev. 24:449-467.

FIG. 6. Predicted topology of the B. anthracis FtsX, generated using program software available at the website for Center for Biological Sequence Analysis of the Technical University of Denmark. Negatively- and positively-charged amino acids are shaded, and the negatively-charged amino acids are designated with an asterisk.

FIG. 7. B. anthracis ftsX mutant strain is resistant to CXCL10. Susceptibility to human CXCL10 (48 ug/ml for 6 hr) was tested using an Alamar Blue assay. Strains tested were: the transposon mutagenesis library, TNX18 isolated from the screen, and the B. anthracis ftsX mutant strain (with ftsX deleted; this strain is also designated in the text as 4ftsX strain). Both TNX18 and the ftsX mutant strain exhibited resistance to CXCL10.

FIG. 8. The B. anthracis ftsX mutant is also resistant to CXCL9 and CXCL11. Susceptibilities to human CXCL9 and CXCL11 (48 ug/ml for 6 hr each) were tested using an Alamar Blue assay. Strains tested were: B. anthracis Sterne strain 7702 parent strain, transposon mutagenesis library, TNX18, and the ftsX mutant. TNX18 and the ftsX mutant were resistant to CXCL9. All strains were resistant to CXCL11, which is consistent with the less effective antimicrobial activity observed for human CXCL11.

FIG. 9. Neutralization of CXCL9, CXCL9/CXCL10, or CXCL9/10/11 but not CXCR3 renders C57BL/6 mice susceptible to B. anthracis infection. C57BL/6 mice received injections of anti-CXCL9, CXCL10, and/or CXCL11 antibodies or anti-CXCR3 antibodies or control goat serum, as indicated in the figure, one day prior to intranasal inoculation with B. anthracis Sterne strain spores and then daily throughout the experiment. Mice were monitored for survival over an 18-day period. *p-value<0.05; **p-value<0.01 compared to spore-inoculated animals that received control goat serum.

FIGS. 10A & 10B Susceptibility of B. anthracis Sterne strain 7702 spores to CXCL10. By treating B. anthracis cultures with an interferon-inducible (ELR−) CXC chemokine in the presence and absence of a heat treatment, one can determine the effectiveness of the interferon-inducible (ELR−) CXC chemokine on spore viability. Thererfore, CFU counts were determined for B. anthracis in the presence and absence of heat treatment at 65° C. for 30 minutes. Cultures not exposed to heat treatment when plated will indicate the number of vegetative and viable spores that were present in the culture, whereas the heat treated culture will only produce CFUs representative of the number of viable spores that were in the culture. As shown in FIG. 10A, treatment with human CXCL9, CXCL10 or CXCL11 (48 ug/ml each for 6 hr) reduced (CXCL11) or eliminated (CXCL9, CXCL10) vegetative outgrowth and disrupted spore germination (CXCL9, CXCL 10). Similar results were obtained for murine interferon-inducible (ELR−) CXC chemokines (see FIG. 10B using an Alamar Blue assay).

FIG. 11A-11C. Susceptibility of exponential versus stationary phase B. anthracis Sterne strain 7702 to CXCL10. Overnight cultures were either diluted back in fresh medium and grown to exponential phase prior to addition of buffer control or CXCL10 at 8 μg/ml (ie, ˜EC₅₀ value; for exponential phase organisms as shown in FIG. 12B below) or used directly from overnight cultures by spinning down, reconstituting in same volume fresh medium plus buffer control or CXCL10 at 8 μg/ml. Aliquots were plated out for CFU determination after an incubation of 30 min or 1 hr. The data from exponential phase B. anthracis are shown in FIG. 11A and data from stationary phase B. anthracis are shown in FIG. 11B. A concentration curve for CXCL10 against stationary phase organisms is shown in (FIG. 11C) with an EC₅₀ value determined to be 0.33 +/−0.05 μg/ml. Each experiment was performed 3 separate times in triplicates. n.d., not detected.

FIG. 12A provides a graph showing the growth curve of ΔftsX compared to the parent B. anthracis Sterne strain 7702 (FIG. 12A). Growth curves were generated using an Alamar Blue assay; RFU=relative fluorescence units.

FIG. 12B provides a graph showing resistance of the B. anthracis ΔftsX strain to CXCL10-mediated killing compared to B. anthracis Sterne strain 7702 (FIG. 12B; designated “7702 wt” in graph). Susceptibility to CXCL10 was determined by a 1 hr incubation with 0-24 ug/ml CXCL10; viability was determined using an Alamar Blue assay; n=3-5 expts.

FIG. 13 Susceptibility of E. coli multi-drug resistant clinical isolate to CXCL10. A carbapenamase-producing E. coli clinical isolate (resistant to penicillins, cephalosporins, carbapenems) was incubated with buffer only or CXCL10 (48 μg/ml) for 3 hrs. Aliquots were plated for overnight CFU determination. **p-value<0.01 compared to buffer-treated control.

FIG. 14 Resistance of E. coli ΔftsEX strain to CXCL10. E. coli wildtype (WT) or ΔftsEXwas incubated with 24 μg/ml CXCL10 for 3 hrs. Aliquots were plated for CFU determination. Data represent log10 kill compared to untreated strain control. ***p-value<0.001 compared to respective untreated strain control.

### DETAILED DESCRIPTION

Abbreviations and Acronyms

diplicolinic acid (DPA)

monokine-induced by interferon-γ (CXCL9)

interferon-γ inducible protein of 10 kd (CXCL10)

interferon-inducible T-cell-activated chemokine (CXCL11)

monocyte chemotactic protein-1 (CCL2); RANTES (CCL5)

half maximal effective concentration (EC50)

transmission electron microscopy (TEM)

In Vivo Imaging System (IVIS)

brain-heart infusion (BHI)

phosphate buffer (PB)

charge coupled device (CCD)

colony forming unit (CFU)

interferon (IFN)

membrane-spanning domain (MSD)

nucleotide binding protein (NBP)

substrate binding protein (SBP)

toll-like receptor (TLR)

glutamic acid-leucine-arginine motif (ELR motif)

Definitions

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about” as used herein means greater or lesser than the value or range of values stated by 10 percent, but is not intended to designate any value or range of values to only this broader definition.

A disease or disorder is “alleviated” if the severity of a symptom of the disease, condition, or disorder, or the frequency with which such a symptom is experienced by a subject, or both, are reduced.

As used herein, the term “subject” refers to an individual (e.g., human, animal, or other organism) to be treated by the methods or compositions of the present invention. Subjects include, but are not limited to, mammals (e.g., murines, simians, equines, bovines, porcines, canines, felines, and the like), and includes humans. In the context of the invention, the term “subject” generally refers to an individual who will receive or who has received treatment for a condition characterized by the presence of bacteria (e.g., Bacillus anthracis (e.g., in any stage of its growth cycle), or in anticipation of possible exposure to bacteria. As used herein, the terms “subject” and “patient” are used interchangeably, unless otherwise noted.

As used herein, the terms “neutralize” and “neutralization” when used in reference to bacterial cells or spores (e.g. B. anthracis cells and spores) refers to a reduction in the ability of the spores to germinate and/or cells to proliferate.

As used herein the term “bacterial spore” or “spore” is used to refer to any dormant, non-reproductive structure produced by some bacteria (e.g., Bacillus and Clostridium) in response to adverse environmental conditions.

As used herein, the term “treating a surface” refers to the act of exposing a surface to one or more compositions of the present invention. Methods of treating a surface include, but are not limited to, spraying, misting, submerging, wiping, and coating. Surfaces include organic surfaces (e.g., food products, surfaces of animals, skin, etc.) and inorganic surfaces (e.g., medical devices, countertops, instruments, articles of commerce, clothing, etc.).

As used herein, the term “therapeutically effective amount” refers to the amount that provides a therapeutic effect, e.g., an amount of a composition that is effective to treat or prevent pathological conditions, including signs and/or symptoms of disease, associated with a pathogenic organism infection (e.g., germination, growth, toxin production, etc.) in a subject.

The terms “bacteria” and “bacterium” refer to all prokaryotic organisms, including those within all of the phyla in the Kingdom Procaryotae. As used herein, the term “microorganism” refers to any species or type of microorganism, including but not limited to, bacteria, archaea, fungi, protozoans, mycoplasma, and parasitic organisms.

As used herein the term “colonization” refers to the presence of bacteria in a subject that are either not found in healthy subjects, or the presence of an abnormal quantity and/or location of bacteria in a subject relative to a healthy patient.

The term “stationary growth phase” as used herein defines the growth characteristics of a given population of microorganisms. During a stationary growth phase the population of bacteria remains stable with the rate of bacterial division being approximately equal to the rate of bacterial death. This may be due to increased generation time of the bacteria. Accordingly “stationary phase bacteria” are bacteria that are in a stationary growth phase. “Exponential phase bacteria” are bacteria that are rapidly proliferating at a rate wherein the population approximately doubles with each round of division. When the growth rate (number of cells vs. time) of exponential phase bacteria is graphed, the plotted data produces an exponential or logarithmic curve.

As used herein a “multi-drug resistant” microorganism or bacteria is an organism that has an enhanced ability, relative to non-resistant strains, to resist distinct drugs or chemicals (of a wide variety of structure and function) targeted at eradicating the organism. Typically the term refers to resistance to at least 3 classes of antibiotics.

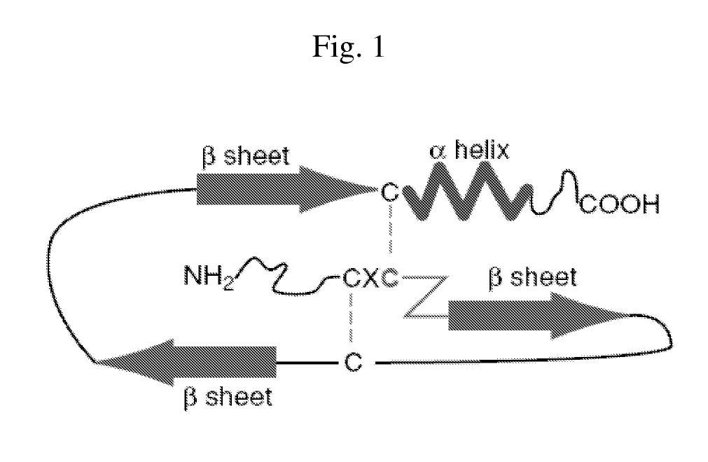

Chemokines are small proteins secreted by cells that have the ability to induce directed chemotaxis in responsive cells. As used herein the term “interferon-inducible (ELR−) CXC chemokine” refers to a chemokine protein, or corresponding peptidomimetic, having a motif of four conserved cysteine residues, the first two of which are separated by a non-conserved amino acid (thus constituting the Cys-X-Cys or ‘CXC’ motif; see FIG. 1) and devoid of a three amino acid sequence, glutamic acid-leucine-arginine (the ‘ELR’ motif), immediately proximal to the CXC sequence. Examples of interferon-inducible (ELR−)CXC chemokines include human CXCL9 (SEQ ID NO: 1), murine CXCL9 (SEQ ID NO: 2), human CXCL10 (SEQ ID NO: 4), murine CXCL10 (SEQ ID NO: 5), human CXCL11 (SEQ ID NO: 7) and murine CXCL11 (SEQ ID NO: 8). CXCL9, CXCL10 and CXCL11 are potently induced by both type 1 and type 2 interferons (IFN-α/β and IFN-γ, respectively).

As used herein the term “lipid vesicle” refers to any spherical shaped structure formed from amphipathic lipids that surround and enclose an interior space. The term lipid vesicle encompasses both micelles as well as liposomes. A micelle is an aggregate of amphipathic lipids with the hydrophilic “head” regions in contact with surrounding solvent, sequestering the hydrophobic tail regions in the micelle centre. A liposome as used herein refers to lipid vesicles comprised of one or more concentrically ordered lipid bilayers encapsulating an aqueous phase. Suitable vesicle-forming lipids may be selected from a variety of amphiphatic lipids, typically including phospho lipids such as phosphatidylcho line (PC) and, sphingo lipids such as sphingomyelin.

As used herein, the term “adjuvant” as used herein refers to an agent which enhances the pharmaceutical effect of another agent.

As used herein, “amino acids” are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

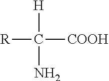

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, charged residues and their amides:
Asp, Asn, Glu, Gln, His, Arg, Lys;

III. Large, aliphatic, nonpolar residues:
Met Leu, Ile, Val, Cys

IV. Large, aromatic residues:
Phe, Tyr, Trp

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be derived from natural sources or from recombinant sources and may be intact immunoglobulins, or immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antimicrobial agent”, as used herein, refers to any entity that exhibits antimicrobial activity, i.e. the ability to inhibit the growth and/or kill bacteria, including for example the ability to inhibit growth or reduce viability of bacteria by at least about 30%, at least about 40%, at least about 50%, at least about 60%, at least about 70% or more than 70%, as compared to bacteria not exposed to the antimicrobial agent. The antimicrobial agent can exert its effect either directly or indirectly and can be selected from a library of diverse compounds, including for example antibiotics. For example, various antimicrobial agents act, inter alia, by interfering with (1) cell wall synthesis, (2) plasma membrane integrity, (3) nucleic acid synthesis, (4) ribosomal function, and (5) folate synthesis. One of ordinary skill in the art will appreciate that a number of “antimicrobial susceptibility” tests can be used to determine the efficacy of a candidate antimicrobial agent.

As used herein, the term “antisense oligonucleotide” means a nucleic acid polymer, at least a portion of which is complementary to a nucleic acid which is present in a normal cell or in an affected cell. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (U.S. Pat. No: 5,034,506; Nielsen et al., 1991, Science 254: 1497). “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

As used herein, the term “biologically active fragments” or “bio active fragment” of the polypeptides encompasses natural or synthetic portions of the full-length protein that are capable of specific binding to their natural ligand or of performing the function of the protein.

A “pathogenic” cell is a cell which, when present in a tissue, causes or contributes to a disease or disorder in the animal in which the tissue is located (or from which the tissue was obtained).

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

Similarly, it is known that a cytosine residue of a first nucleic acid strand is capable of base pairing with a residue of a second nucleic acid strand which is antiparallel to the first strand if the residue is guanine A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “cell” and “cell line,” as used herein, may be used interchangeably. All of these terms also include their progeny, which are any and all subsequent generations. It is understood that all progeny may not be identical due to deliberate or inadvertent mutations.

The terms “cell culture” and “culture,” as used herein, refer to the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

The phrases “cell culture medium,” “culture medium” (plural “media” in each case) and “medium formulation” refer to a nutritive solution for cultivating cells and may be used interchangeably.

A “conditioned medium” is one prepared by culturing a first population of cells or tissue in a medium, and then harvesting the medium. The conditioned medium (along with anything secreted into the medium by the cells) may then be used to support the growth or differentiation of a second population of cells.

The term “complex”, as used herein in reference to proteins, refers to binding or interaction of two or more proteins. Complex formation or interaction can include such things as binding, changes in tertiary structure, and modification of one protein by another, such as phosphorylation.

A “compound”, as used herein, refers to any type of substance or agent that is commonly considered a chemical, drug, or a candidate for use as a drug, as well as combinations and mixtures of the above. The term compound further encompasses molecules such as peptides and nucleic acids.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

As used herein, a “derivative” of a compound refers to a chemical compound that may be produced from another compound of similar structure in one or more steps, as in replacement of H by an alkyl, acyl, or amino group.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

The terms “formula” and “structure” are used interchangeably herein.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “inhibit,” as used herein, refers to the ability of a compound or any agent to reduce or impede a described function or pathway. For example, inhibition can be by at least 10%, by at least 25%, by at least 50%, and even by at least 75%.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated” compound/moiety is a compound/moeity that has been removed from components naturally associated with the compound/moiety. For example, an “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “modulate”, as used herein, refers to changing the level of an activity, function, or process. The term “modulate” encompasses both inhibiting and stimulating an activity, function, or process.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “Oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “peptide” typically refers to short polypeptides.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

A “recombinant polypeptide” is one which is produced upon expression of a recombinant polynucleotide.

A peptide encompasses a sequence of 2 or more amino acids wherein the amino acids are naturally occurring or synthetic (non-naturally occurring) amino acids.

The term “linked” or like terms refers to a connection between two entities. The linkage may comprise a covalent, ionic, or hydrogen bond or other interaction that binds two compounds or substances to one another.

As used herein the term “peptidomimetic” refers to a chemical compound having a structure that is different from the general structure of an existing peptide, but that functions in a manner similar to the existing peptide, e.g., by mimicking the biological activity of that peptide. Peptidomimetics typically comprise naturally-occurring amino acids and/or unnatural amino acids, but can also comprise modifications to the peptide backbone. For example a peptidomimetic may include one or more of the following modifications:

1. peptides wherein one or more of the peptidyl —C(O)NR— linkages (bonds) have been replaced by a non-peptidyl linkage such as a —CH2-carbamate linkage (—CH2OC(O)NR—), a phosphonate linkage, a —CH2-sulfonamide (—CH2—S(O)2NR—) linkage, a urea (—NHC(O)NH—) linkage, a —CH2-secondary amine linkage, an azapeptide bond (CO substituted by NH), or an ester bond (e.g., depsipeptides, wherein one or more of the amide (—CONHR—) bonds are replaced by ester (COOR) bonds) or with an alkylated peptidyl linkage (—C(O)NR—) wherein R is C1-C4 alkyl;

2. peptides wherein the N-terminus is derivatized to a —NRR1 group, to a —NRC(O)R group, to a —NRC(O)OR group, to a —NRS(O)2R group, to a —NHC(O)NHR group where R and R1 are hydrogen or C1-C4 alkyl with the proviso that R and R1 are not both hydrogen;

3. peptides wherein the C terminus is derivatized to —C(O)R2 where R2 is selected from the group consisting of C1-C4 alkoxy, and —NR3R4 where R3 and R4 are independently selected from the group consisting of hydrogen and C1-C4 alkyl;

4. modification of a sequence of naturally-occurring amino acids with the insertion or substitution of a non-peptide moiety, e.g. a retroinverso fragment.

The term “permeability,” as used herein, refers to transit of fluid, cell, or debris between or through cells and tissues.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds,” as used herein, is meant a compound which recognizes and binds a specific protein, but does not substantially recognize or bind other molecules in a sample, or it means binding between two or more proteins as in part of a cellular regulatory process, where said proteins do not substantially recognize or bind other proteins in a sample. The term “standard,” as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “symptom,” as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

As used herein an “amino acid modification” refers to a substitution, addition or deletion of an amino acid, and includes substitution with, or addition of, any of the 20 amino acids commonly found in human proteins, as well as unusual or non-naturally occurring amino acids. Commercial sources of unusual amino acids include Sigma-Aldrich (Milwaukee, Wis.), ChemPep Inc. (Miami, Fla.), and Genzyme Pharmaceuticals (Cambridge, Mass.). Unusual amino acids may be purchased from commercial suppliers, synthesized de novo, or chemically modified or derivatized from naturally occurring amino acids. Amino acid modifications include linkage of an amino acid to a conjugate moiety, such as a hydrophilic polymer, acylation, alkylation, and/or other chemical derivatization of an amino acid.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substitutions may be designed based on, for example, the model of Dayhoff, et al. (in Atlas of Protein Sequence and Structure 1978, Nat'l Biomed. Res. Found., Washington D.C.).

Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., Science 247:1306-1310 (1990).

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride, or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the a-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resincoupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

### Embodiments

In accordance with one embodiment compositions and methods are provided for neutralizing pathogenic organisms. More particularly, applicants have found that interferon-inducible ELR− CXC chemokines have efficacy against pathogenic bacteria including Bacillus anthraci. In accordance with one embodiment a composition is provided for neutralizing pathogenic bacteria in all growth phases including sporulated forms. The compositions can be formulated for treatment of external surfaces including hard surfaces such as, medical equipment and medical devices, or the compositions can be formulated for topical or internal administration to subjects, including humans.

In accordance with one embodiment a composition is provided comprising a non-native peptide, or a peptidomimetic derivative, comprising a sequence selected from the group consisting of SEQ ID NO: 3, SEQ ID NO: 6 and SEQ ID NO: 9 or a sequence that differs from SEQ ID NO: 3, SEQ ID NO: 6 or SEQ ID NO: 9 by 1, 2, 3, 4 or 5 amino acids. In one embodiment the peptide differs from SEQ ID NO: 3, SEQ ID NO: 6 or SEQ ID NO: 9 by 1, 2, 3, 4 or 5 conservative amino acid substitutions. In accordance with one embodiment a composition is provided comprising a peptide, or a peptidomimetic derivative, comprising a sequence selected from the group consisting of SEQ ID NO: 3, SEQ ID NO: 6 and SEQ ID NO: 9. In a further embodiment a composition is provided comprising a non-native peptide, or a peptidomimetic derivative thereof, comprising a sequence selected from the group consisting of SEQ ID NO: 6, SEQ ID NO: 13 or SEQ ID NO: 16.

In another embodiment the non-native peptide, or peptidomimetic derivative thereof, comprises a sequence selected from the group consisting of SEQ ID NO: 12, SEQ ID NO: 13 and SEQ ID NO: 14 or a sequence that differs from SEQ ID NO: 12, SEQ ID NO: 13 or SEQ ID NO: 14 by 1, 2, 3, 4 or 5 amino acids. In another embodiment the peptide, or peptidomimetic derivative, comprises a sequence selected from the group consisting of SEQ ID NO: 15, SEQ ID NO: 16 and SEQ ID NO: 17 or a sequence that differs from SEQ ID NO: 15, SEQ ID NO: 16 or SEQ ID NO: 17 by 1, 2, 3, 4 or 5 amino acids. In another embodiment a composition is provided comprising a non-native peptide, or a peptidomimetic derivative, comprising a sequence selected from the group consisting of SEQ ID NO: 1, SEQ ID NO: 4 or SEQ ID NO: 7, and in further embodiment the sequence comprises the amino acid sequence of SEQ ID NO: 4 or SEQ ID NO: 16, or a peptidomimetic derivative thereof.

In one embodiment a composition comprising an interferon-inducible (ELR−) CXC chemokine is provided wherein the chemokine comprises a peptidomimetic derivative or non-native peptide sequence selected from the group consisting of i) SEQ ID NO: 3 or SEQ ID NO: 6 or a peptide having at least 95% amino acid sequence identity with SEQ ID NO: 3 or SEQ ID NO: 6 or SEQ ID NO: 9. In a further embodiment the interferon-inducible (ELR−) CXC chemokine comprises a sequence of SEQ ID NO: 15 or SEQ ID NO: 16. In a further embodiment the interferon-inducible (ELR−) CXC chemokine comprises a peptide sequence that differs from SEQ ID NO: 4 by no more than 1, 2, 3, 4 or 5 amino acid modifications at one or more positions selected from amino acid positions 3, 4, 6, 7, 9, 13, 15, 19, 22, 25, 31, 34, 36, 37, 38, 41, 42, 44, 45, 46, 55, 56, 69, 70, 72, 81, 86, 89, 92 or 97. In one embodiment the amino acid modifications are amino acid substitution, and in one embodiment the substitutions are conservative amino acid substitutions.

In some embodiments, the peptide of the present disclosures comprises a non-native amino acid sequence which has at least 75%, 80%, 85%, 90% or 95% sequence identity to an amino acid sequence of SEQ ID NO: 3, SEQ ID NO: 6 or SEQ ID NO: 9, or a peptidomimetic derivative of SEQ ID NO: 3, SEQ ID NO: 6 or SEQ ID NO: 9. The statement that the peptide is a non-native is intended to exclude the native peptides of SEQ ID NO: 1, SEQ ID NO: 4 and SEQ ID NO: 7. In some embodiments, the peptide of the present disclosures comprises a non-native amino acid sequence which has at least 75%, 80%, 85%, 90% or 95% sequence identity to an amino acid sequence of SEQ ID NO: 15, SEQ ID NO: 16 or SEQ ID NO: 17, or peptidomimetic derivative of SEQ ID NO: 15, SEQ ID NO: 16 or SEQ ID NO: 17. In some embodiments, the peptide of the present disclosure comprises a non-native amino acid sequence which has at least 75%, 80%, 85%, 90% or 95% sequence identity to an amino acid sequence of SEQ ID NO: 1, SEQ ID NO: 4 or SEQ ID NO: 7, or a peptidomimetic derivative of SEQ ID NO: 1, SEQ ID NO: 4 or SEQ ID NO: 7. In some embodiments, the peptide of the present disclosure comprises an amino acid sequence which has at least a 90% amino acid sequence identity with SEQ ID NO: 1, SEQ ID NO: 4 or SEQ ID NO: 7, with the proviso that the peptide is not SEQ ID NO: 1, SEQ ID NO: 4 or SEQ ID NO: 7. In some embodiments, the peptide of the present disclosure comprises a non-native amino acid sequence which has at least a 95% amino acid sequence identity with SEQ ID NO: 1, SEQ ID NO: 2, SEQ ID NO: 4 or SEQ ID NO: 16. In some embodiments, the peptide of the present disclosure comprises a non-native amino acid sequence which has at least a 95% amino acid sequence identity with SEQ ID NO: 16.

In some embodiments, the peptide of the present disclosures comprises an amino acid sequence which has at least 95% sequence identity to an amino acid sequence of SEQ ID NO: 1, SEQ ID NO: 4 or SEQ ID NO: 7. In some embodiments, the peptide of the present disclosures comprises an amino acid sequence which is at least 70%, at least 80%, at least 85%, at least 90% or has greater than 95% sequence identity to SEQ ID NO: 4. In some embodiments, the amino acid sequence of the presently disclosed peptide which has the above-referenced % sequence identity is the full-length amino acid sequence of the presently disclosed peptide.

In one embodiment an antimicrobial composition is provided comprising two or more interferon-inducible (ELR−) CXC chemokines. In one embodiment the composition comprises a purified first peptide having the sequence of SEQ ID NO; 12 or SEQ ID NO: 15, and a purified second peptide having the sequence of SEQ ID NO: 13 or SEQ ID NO: 16. In one embodiment the composition comprises a non-native first peptide having the sequence of SEQ ID NO: 15, and a non-native second peptide having the sequence of SEQ ID NO: 16.

It is further contemplated that the antimicrobial interferon-inducible (ELR−) CXC chemokines disclosed herein may be used in combination with, or to enhance the activity of, other antimicrobial agents or antibiotics. In one embodiment a composition is provided comprising an interferon-inducible (ELR−) CXC chemokine and a second antimicrobial agent. In one embodiment the second antimicrobial agent is an antibiotic. Combinations of an interferon-inducible (ELR−) CXC chemokine peptide (or other compounds identified by the methods disclosed herein) with other agents may be useful to allow antibiotics to be used at lower doses responsive to toxicity concerns, to enhance the activity of antibiotics whose efficacy has been reduced or to effectuate a synergism between the components such that the combination is more effective than the sum of the efficacy of either component independently.

In some embodiments, the antimicrobial agent is a quinolone antimicrobial agent, including for example but not limited to, ciprofloxacin, levofloxacin, and ofloxacin, gatifloxacin, norfloxacin, lomefloxacin, trovafloxacin, moxifloxacin, sparfloxacin, gemifloxacin, pazufloxacin or variants or analogues thereof. In some embodiments, the second antimicrobial agent is ofloxacin or variants or analogues thereof.

In some embodiments, the second antimicrobial agent is an aminoglycoside antimicrobial agent, including for example but not limited to, amikacin, gentamycin, tobramycin, netromycin, streptomycin, kanamycin, paromomycin, neomycin or variants or analogues thereof In some embodiments, the second antimicrobial agent is gentamicin or variants or analogues thereof.

In some embodiments, the second antimicrobial agent is a beta-lactam antibiotic antimicrobial agent, including for example but not limited to, penicillin, ampicillin, penicillin derivatives, cephalosporins, monobactams, carbapenems, beta-lactamase inhibitors or variants or analogues thereof In some embodiments, the second antimicrobial agent is ampicillin or variants or analogues thereof In accordance with one embodiment the second antimicrobial agent is selected from a group consisting of penicillin, ampicillin, penicillin derivatives, cephalosporins, monobactams, carbapenems, or beta-lactamase inhibitors.

The compositions disclosed herein may include additional components that enhance their efficacy based on their desired use. In one embodiment the compositions are formulated as a pharmaceutical composition. The pharmaceutical compositions can be prepared for systemic (parenteral), inhalational (or inhaled), and topical applications using formulations and techniques known to those skilled in the art. Such pharmaceutical compositions include one or more isolated or purified interferon-inducible (ELR−) CXC chemokines, or pharmaceutically acceptable salts thereof, and a pharmaceutically acceptable carrier.

The pharmaceutical composition can comprise any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

In one embodiment the interferon-inducible (ELR−) CXC chemokine may be coupled, bonded, bound, conjugated, or chemically-linked to one or more agents via linkers, polylinkers, or derivatized amino acids. In accordance with one embodiment the composition further comprises a lipid vesicle delivery vehicle. In one embodiment the lipid vesicle is a liposome or micelle. Suitable lipids for liposomal and/or micelle formulation include, without limitation, monoglycerides, diglycerides, sulfatides, lysolecithin, phospholipid, saponin, bile acids, and the like. The preparation of liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, the disclosures of which are incorporated herein by reference. In accordance with one embodiment a composition is provided comprising an interferon-inducible (ELR−) CXC chemokine and a lipid vesicle, wherein the interferon-inducible (ELR−) CXC chemokine is encapsulated within the lipid vesicle, or linked to the surface of said lipid vesicle. In a further embodiment the composition may include additional active agents encapsulated or linked to the surface of the lipid vesicle delivery vehicle, including for example an anti-microbial agent such as an antibiotic. In one embodiment the lipid vesicle is a liposome, and in a further embodiment the liposome comprises interferon-inducible (ELR−) CXC chemokines linked to the exterior surface of the liposome. In one embodiment the interferon-inducible (ELR−) CXC chemokines are covalently bound to the exterior surface of the liposome, optionally with additional active antimicrobial agents encapsulated within or linked to the exterior surface of the liposome.

In some embodiments, the pharmaceutical composition comprises an interferon-inducible (ELR−) CXC chemokine and an antibiotic. Antibiotics suitable for use in accordance with the present description include for example, but are not limited to, a lantibiotic (e.g. nisin or epidermin), almecillin, amdinocillin, amikacin, amoxicillin, amphomycin, amphotericin B, ampicillin, azacitidine, azaserine, azithromycin, azlocillin, aztreonam; bacampicillin, bacitracin, benzyl penicilloyl-polylysine, bleomycin, candicidin, capreomycin, carbenicillin, cefaclor, cefadroxil, cefamandole, cefazo line, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, eveminomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezo lid, linocomycin, loracarbef, magainin, meclocycline, meropenem, methacycline, methicillin, mezlocillin, minocycline, mitomycin, moenomycin, moxalactam, moxifloxacin, mycophenolic acid, nafcillin, natamycin, neomycin, netilmicin, niphimycin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamycin, BMS-284,756, L-749,345, ER-35,786, S-4661, L-786,392, MC-02479, PepS, RP 59500, and TD-6424. In some embodiments, two or more antimicrobial agents (e.g., a composition comprising an interferon-inducible (ELR−) CXC chemokine and an antibiotic) may be used together or sequentially. In some embodiments, another antibiotic may comprise bacteriocins, type A lantibiotics, type B lantibiotics, liposidomycins, mureidomycins, alanoylcholines, quinolines, eveminomycins, glycylcyclines, carbapenems, cephalosporins, streptogramins, oxazolidonones, tetracyclines, cyclothialidines, bioxalomycins, cationic peptides, and/or protegrins.

In one embodiment the antibiotics that are combined with the interferon-inducible (ELR−) CXC chemokine include but are not limited to penicillin, ampicillin, amoxycillin, vancomycin, cycloserine, bacitracin, cephalolsporin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraminosalicylic acid, and ethambutol. In some embodiments, the antibiotic comprises one or more anti-anthrax agents (e.g., an antibiotic used in the art for treating B. anthracis (e.g., penicillin, ciprofloxacin, doxycycline, erythromycin, and vancomycin)).

In one embodiment a kit is provided for neutralizing pathogenic organisms. In one embodiment the kit comprises an interferon-inducible (ELR−) CXC chemokine (as disclosed herein) and additional known antimicrobial agents, including one or more antibiotics. In a further embodiment the kit comprises a type 1 and/or type 2 interferons (e.g., IFN-α/β and IFN-γ, respectively).

### Neutralizing Stationary Phase Bacteria

Many antibiotics are only poorly effective against non-growing or stationary phase bacteria. During the stationary period bacterial cells frequently have a thicker peptidoglycan cell wall and typically have differences in metabolism and protein synthesis. Many complications that arise during the course of treating bacterial infections are due to stationary phase or dormant bacteria, which as noted above resist conventional antibiotic treatments. The formation of bio films on temporary (e.g., catheters) or more permanent implants and the colonizations seen in patients afflicted with certain diseases cannot be effectively treated with the antimicrobial agents currently available. In terms of bacterial colonization and diseases that can arise from it, the airways and the GI tract are the major areas affected. The presence of inappropriate bacterial colonizations is believed to cause complications associated with inflammatory bowel diseases (including ulcerative colitis and Crohn's disease) and irritable bowel syndrome. In addition with regards to the airways alone, the major diseases that can arise from, or can be exacerbated by, bacterial colonization include: sinus infections, respiratory infections such as pneumonia (this is especially applicable to ventilator-associated pneumonias but also applies to community-acquired pneumonias), chronic obstructive pulmonary disease (COPD), and cystic fibrosis (CF).

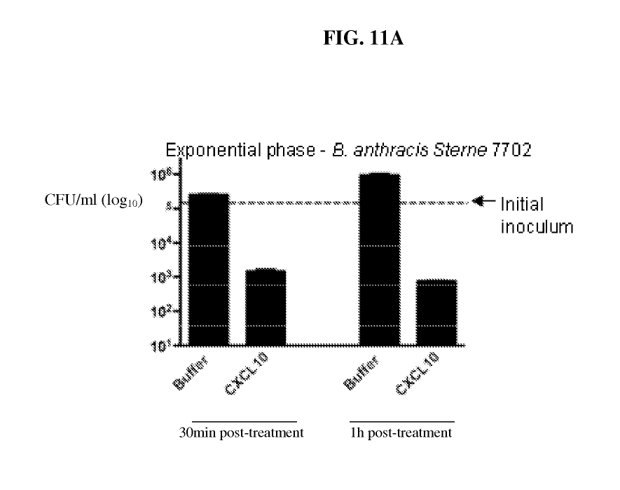

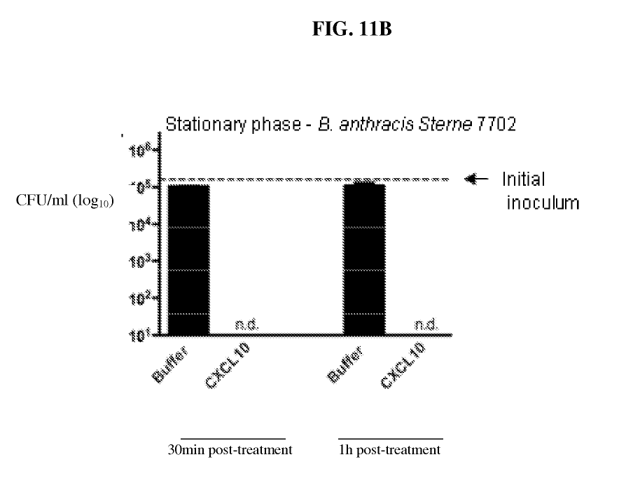

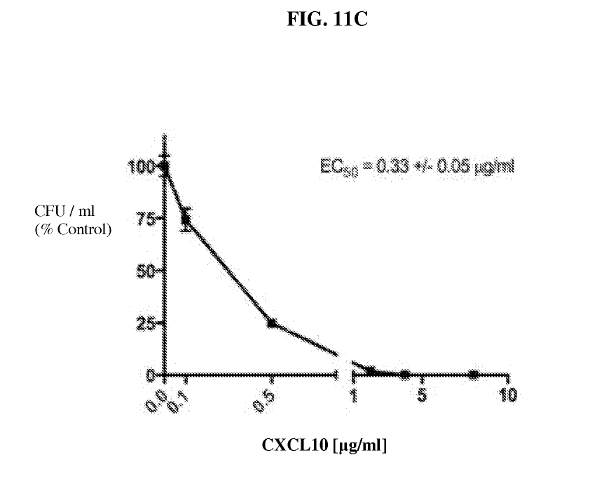

Surprisingly, applicants have discovered that the interferon-inducible (ELR−) CXC chemokines have activity in neutralizing stationary phase bacteria as well as actively growing bacteria (see FIGS. 11A-11C). In accordance with one embodiment a method is provided for neutralizing prokaryotic pathogenic organisms that have colonized a host organism and have entered into a stationary growth phase. It is also anticipated that the interferon-inducible (ELR−) CXC chemokine containing compositions may have efficacy in neutralizing biofilms. The method comprises the step of contacting the pathogenic organisms with a composition comprising an interferon-inducible (ELR−) CXC chemokine

In accordance with one embodiment the method comprises the steps of contacting the pathogenic organisms with an effective amount of a peptide selected from the group consisting of i) CXCL-9 (SEQ ID NO: 1), CXCL-10 (SEQ ID NO: 4) or CXCL 11 (SEQ ID NO: 7), ii) a peptide fragment of CXCL-9, CXCL-10 or CXCL 11, or a peptide having at least 90% amino acid sequence identity with i) or ii). In one embodiment the peptide comprises the sequence of SEQ ID NO: 3, SEQ ID NO: 6 and SEQ ID NO: 9, or a peptidomimetic derivative thereof. In another embodiment the peptide comprises a sequence selected from the group consisting of i) SEQ ID NO: 3 or SEQ ID NO: 6 or a peptide having at least 95% amino acid sequence identity with SEQ ID NO: 3 or SEQ ID NO: 6 or SEQ ID NO: 9. In one embodiment the peptide comprises the sequence of SEQ ID NO: 15 or SEQ ID NO: 16. In another embodiment the peptide comprises a peptide sequence that differs from SEQ ID NO: 4 by no more than 1, 2, 3, 4 or 5 amino acid modifications at positions selected from amino acid positions 3, 4, 6, 7, 9, 13, 15, 19, 22, 25, 31, 34, 36, 37, 38, 41, 42, 44, 45, 46, 55, 56, 69, 70, 72, 81, 86, 89, 92 or 97. In one embodiment the amino acid modifications are amino acid substitutions including for example conservative amino acid substitutions. In one embodiment the peptide sequence differs from SEQ ID NO: 4 by 1, 2, 3, 4 or 5 amino acid modifications at positions selected from amino acid positions 3, 4, 6, 7, 9, 13, 15, 19, 22, 25, 31, 34, 36, 37, 38, 41, 42, 44, 45, 46, 55, 56, 69, 70, 72, 81, 86, 89, 92 or 97.

Since interferons are known to induce expression of native CXCL9, CXCL10 and CXCL11, in one embodiment the method of treatment comprises the co-administration of one or more interferons, including for example interferon-alpha, interferon-beta and/or interferon-gamma as an adjuvant to promote production of native CXCL9, CXCL10 and CXCL11 chemokines in vivo. Co-aministration can be accomplished by simultaneously administering the chemokine and the interferon, or the two active agents can be administered one after the other within 1, 2, 3, 4, 5, 6, 12, 24 or 48 hours of each other.

The pathogenic organisms are placed in contact using an appropriate route of administration. For example, for treating skin, the composition can be formulated as a topical cream, ointment or in a rinsing solution. Such composition can be used sterilize external body parts that may have come in contact with pathogenic organisms such as Bacillus anthracis. Alternatively, formulations for oral administration can be prepared for treating bacterial colonization of the digestive tract. In another embodiment the composition can be formulated as an aerosol for administration to the lungs and air pathways of a subject. Such formulations can be prepared using standard formulations and techniques known to the skilled practitioner.

The interferon-inducible (ELR−) CXC chemokine compositions will be administered in an amount effective to neutralize the bacteria. An “effective” amount or a “therapeutically effective amount” of the interferon-inducible (ELR−) CXC chemokine refers to a nontoxic but sufficient amount of the compound to provide the desired effect. The amount that is “effective” will vary based on the organism to be neutralized, whether an external surface is to be treated or whether the composition is to be administered as a pharmaceutical, the mode of administration, and the like. Thus, it is not always possible to specify an exact “effective amount.” However, an appropriate “effective” amount in any individual case may be determined by one of ordinary skill in the art using routine experimentation.

In one embodiment the method comprises contacting the bacteria with an interferon-inducible (ELR−) CXC chemokine at a concentration of about 1 to about 100 μg/ml, about 1 to about 75 μg/ml, about 1 to about 50 μg/ml, 1 to about 30 μg/ml, 1 to about 15 μg/ml, 2 to about 10 μg/ml, 4 to about 8 μg/ml, 6 to about 10 μg/ml or about 8 μg/ml. Typically the bacteria are contacted with an effective amount of the interferon-inducible (ELR−) CXC chemokines for a time ranging from 1 to 6, 2 to 8, 4 to 12 or 12 to 24 hours.

In accordance with one embodiment the administered anti-microbial composition comprises an interferon-inducible (ELR−) CXC chemokine having a peptide sequence selected from the group consisting of SEQ ID NO: 3, SEQ ID NO: 6 and SEQ ID NO: 9, or a peptidomimetic derivative thereof. In one embodiment such a composition is used to neutralize and/or kill both active and stationary phase pathogenic bacteria, including for example pathogenic organism is selected from the group consisting of Streptococcus pneumoniae, Staphylococcus aureus, Moraxella catarrhalis, Hemophilus influenzae, Enterobacteriaceae, Pseudomonas aeruginosa, Stenotrophomonas maltophilia, Streptococcus viridans, Neisseria spp., and Corynebacterium spp.

Several disease states are associated with large populations of stationary phase bacteria, and currently there are not effective treatments for removing such bacterial colonizations of patients. These diseases include pneumonia (this is especially applicable to ventilator-associated pneumonias but also applies to community-acquired pneumonias), and pulmonary infections associated with, chronic obstructive pulmonary disease (COPD), cystic fibrosis (CF), wherein populations of bacteria remain resident in the host organism. Major contributors to pathogenic infections of patient airways include both Gram-positive and Gram-negative bacteria and include, but are not limited to the following as major contributors Gram-positive cocci such as Streptococcus and Staphylococcus species, including for example Streptococcus pneumoniae and Staphylococcus aureus, Gram-negative cocci such as Moraxella catarrhalis, Gram-negative rods such as Hemophilus influenzae, Enterobacteriaceae, and Pseudomonas aeruginosa. Additional organisms that might play a role in immunocompromised hosts (in addition to the above listed organisms) may include Streptococcus viridans group, coagulase-negative staphylococci, Neisseria spp., and Corynebacterium spp. Yeast such as Candida spp. can also play a role. In cystic fibrosis patients, Stenotrophomonas maltophilia is an ever more problematic Gram negative pathogen that colonizes the airways along with the above listed organisms (especially Pseudomonas aeruginosa and S. aureus). One aspect of the present disclosure is the use of the interferon-inducible (ELR−) CXC chemokines to treat subjects suffering from a disease or condition that is exacerbated by the presence of inappropriate bacteria such as those listed above.

In accordance with one embodiment the method of treating a pathogenic colonization of a patient is provided wherein a composition comprising an interferon-inducible (ELR−) CXC chemokine peptide, or peptidomimetic derivative thereof is administered to the patient. In one embodiment the composition comprises a peptide selected from the group i) SEQ ID NO: 1, SEQ ID NO: 2, SEQ ID NO: 4 SEQ ID NO: 5, SEQ ID NO: 7 and SEQ ID NO: 8, ii) a peptide fragment of SEQ ID NO: 1, SEQ ID NO: 2, SEQ ID NO: 4, SEQ ID NO: 5, SEQ ID NO: 7 and SEQ ID NO: 8, or a peptide having at least 90% amino acid sequence identity with i) or ii). In a further embodiment the composition comprises an interferon-inducible (ELR−) CXC chemokine peptide, or peptidomimetic derivative thereof, wherein the peptide comprises a sequence selected from the group consisting of SEQ ID NO: 1, SEQ ID NO: 2 and SEQ ID NO: 4. In a further embodiment the composition comprises two or three peptides selected from the group consisting of SEQ ID NO: 1, SEQ ID NO: 2, and SEQ ID NO: 4. In one embodiment the composition comprises a peptide comprising the sequence of SEQ ID NO: 1, SEQ ID NO: 2 and SEQ ID NO: 4 or a sequence that is 95% identical in sequence with SEQ ID NO: 1, SEQ ID NO: 2 or SEQ ID NO: 4.

In accordance with one embodiment the method of treating a pathogenic colonization of a patient is provided wherein a composition comprising an interferon-inducible (ELR−) CXC chemokine peptide selected from the group consisting of SEQ ID NO: 15 or SEQ ID NO: 16 is administered to the patient. In accordance with one embodiment the composition comprises the sequence of SEQ ID NO: 4 or a sequence that differs from SEQ ID NO: 4 by 1, 2, 3, 4, 5, 6, 7, 8, 9, or 10 amino acid modifications at positions independently selected from positions 4, 7, 22, 25, 37, 44, 45, 72, 86, 91, 92, 97. In one embodiment the differences represent amino acid substitutions and in one embodiment the substitutions are conservative amino acid substitutions. In one embodiment the peptide sequence differs from SEQ ID NO: 4 by 1, 2, 3, 4 or 5 amino acid modifications at positions selected from amino acid positions 3, 4, 6, 7, 9, 13, 15, 19, 22, 25, 31, 34, 36, 37, 38, 41, 42, 44, 45, 46, 55, 56, 69, 70, 72, 81, 86, 89, 92 or 97.

In one embodiment the compositions further comprise additional anti-microbial agents including, for example, one or more antibiotics. In another embodiment the method comprises administering one or more interferon-inducible (ELR−) CXC chemokine peptides wherein the interferon-inducible (ELR−) CXC chemokine is linked, optionally via covalent bonding and optionally via a linker, to a conjugate moiety. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other.

The peptide can be linked to conjugate moieties via direct covalent linkage by reacting targeted amino acid residues of the peptide with an organic derivatizing agent that is capable of reacting with selected side chains or the N- or C-terminal residues of these targeted amino acids. Reactive groups on the peptide or conjugate include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, the conjugate moieties can be linked to the peptide indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier.

Exemplary conjugate moieties that can be linked to any of the glucagon peptides described herein include but are not limited to a heterologous peptide or polypeptide (including for example, a plasma protein), a targeting agent, an immunoglobulin or portion thereof (e.g. variable region, CDR, or Fc region), a diagnostic label such as a radioisotope, fluorophore or enzymatic label, a polymer including water soluble polymers, or other therapeutic or diagnostic agents.

In accordance with one embodiment a method of treating a pathogenic colonization of a patient is provided wherein a composition comprising the interferon-inducible (ELR−) CXC chemokine linked to a lipid vesicle is administered to a subject in need thereof. In one embodiment the interferon-inducible (ELR−) CXC chemokine is linked to the external surface of the lipid vesicle, and in one embodiment the interferon-inducible (ELR−) CXC chemokine is covalently bound to the lipids comprising the lipid vesicle. In an alternative embodiment the interferon-inducible (ELR−) CXC chemokine is entrapped within the lipid vesicle. In one embodiment the lipid vesicle is a liposome. In a further embodiment the composition comprises additional anti-microbial agents, including for example one or more antibiotics. It is anticipated that the administration of the interferon-inducible (ELR−) CXC chemokine will enhance the efficacy of the known anti-microbial agent. The known anti-microbial agents can be co-administered with the interferon-inducible (ELR−) CXC chemokine either in a single dosage form or the therapeutic agents can be administered sequentially, within 5, 10, 15, 30, 60, 120, 180, 240 minutes or 12, 24 or 48 hours, to one another. In one embodiment the interferon-inducible (ELR−) CXC chemokine is linked to a liposome, optionally with the known anti-microbial agents also linked to the same liposome.

### Neutralizing Multi-Drug Resistant Strains

During the last several decades bacterial resistance has emerged as a new trend, contributing to morbidity and mortality caused by bacterial infections. A troubling percentage of bacterial pathogens causing infections encountered in clinical settings are resistant to some form of antibiotic therapy. Due to the excessive and not always appropriate use of antibiotics in humans and animal feed, bacterial resistance currently constitutes a major public health crisis. The World Health Organization (WHO) reported that drug resistant strains of microbes had a negative impact on their fight against tuberculosis, cholera, diarrhea and pneumonia, which together killed more than ten million people worldwide in 1995.

Multi-drug resistant strains of bacteria such as methicillin-resistant Staphylococcal aureus (MRSA) and vancomycin-resistant enterococci (VRE) were first encountered in hospital settings, but many of them can now be found infecting healthy individuals in larger communities. The spread of VRE is particularly concerning when it is taken into account that vancomycin is generally regarded as the last line of defense in the antibiotic arsenal. Additionally, the extensive use of beta-lactam antibiotics such as penicillin and ampicillin has also resulted in significant numbers of resistant strains among both Gram-positive and Gram-negative bacteria. Furthermore, strains can be deliberately engineered to have multi-drug resistance as part of “weaponization” of wild type strains, including for example Bacillus anthracis.

Currently, the choices for treatment of antibiotic-resistant and multi-drug resistant bacteria are limited in scope even though the molecular mechanisms of resistance are fairly well understood. The four main mechanisms by which microorganisms exhibit resistance to antimicrobials are:

1) Drug inactivation or modification: e.g. enzymatic deactivation of Penicillin G in some penicillin-resistant bacteria through the production of β-lactamases.

2) Alteration of target site: e.g. alteration of PBP—the binding target site of penicillins—in MRSA and other penicillin-resistant bacteria.

3) Alteration of metabolic pathway: e.g. some sulfonamide-resistant bacteria do not require para-aminobenzoic acid (PABA), an important precursor for the synthesis of folic acid and nucleic acids in bacteria inhibited by sulfonamides. Instead, like mammalian cells, they turn to utilizing preformed folic acid.

4) Reduced drug accumulation: by decreasing drug permeability and/or increasing active efflux (pumping out) of the drugs across the cell surface. In many cases, antibiotic-resistant and multi-drug resistant bacteria such as MRSA and VRE encode the antibiotic resistance genes on plasmids. These plasmids can be laterally transferred between bacteria and hence account for the rapid dissemination of antibiotic resistance genes into diverse bacterial populations.

Surprisingly, applicants have found that compositions comprising the interferon-inducible (ELR−) CXC chemokines disclosed herein have efficacy in neutralizing multi-drug resistant bacteria. Accordingly, one aspect of the present disclosure is the use of the interferon-inducible (ELR−) CXC chemokines either alone or in combination with other anitmicrobial agents to neutralize multi-drug resistant bacteria. In one embodiment a method for inhibiting the proliferation of a multi-drug resistant bacteria comprises contacting a multi-drug resistant bacteria with an effective amount of the compound of an interferon-inducible (ELR−) CXC chemokine of the present disclosure.

In accordance with one embodiment the method comprises the steps of contacting the multi-drug resistant organisms with an effective amount of a peptide selected from the group consisting of i) CXCL-9 (SEQ ID NO: 1), CXCL-10 (SEQ ID NO: 4) or CXCL 11 (SEQ ID NO: 7), ii) a peptide fragment of CXCL-9, CXCL-10 or CXCL 11, or a peptide having at least 90% amino acid sequence identity with i) or ii). In one embodiment the peptide comprises the sequence of SEQ ID NO: 3, SEQ ID NO: 6 and SEQ ID NO: 9, or a peptidomimetic derivative thereof In another embodiment the peptide comprises a sequence selected from the group consisting of i) SEQ ID NO: 3 or SEQ ID NO: 6 or a peptide having at least 95% amino acid sequence identity with SEQ ID NO: 3 or SEQ ID NO: 6. In one embodiment the peptide comprises the sequence of SEQ ID NO: 15 or SEQ ID NO: 16. In a further embodiment the multi-drug resistant organisms are contacted with a composition comprising an interferon-inducible (ELR−) CXC chemokine peptide, or peptidomimetic derivative thereof, wherein the peptide comprises a sequence selected from the group consisting of SEQ ID NO: 1, SEQ ID NO: 2 and SEQ ID NO: 4. In a further embodiment the composition comprises two or three peptides selected from the group consisting of SEQ ID NO: 1, SEQ ID NO: 2, and SEQ ID NO: 4. In one embodiment the composition comprises a peptide comprising the sequence of SEQ ID NO: 1, SEQ ID NO: 2, SEQ ID NO: 4 or a sequence that is 95% identical in sequence with SEQ ID NO: 1, SEQ ID NO: 2 or SEQ ID NO: 4. In another embodiment the peptide comprises a peptide sequence that differs from SEQ ID NO: 4 by no more than 1, 2, 3, 4 or 5 amino acid modifications at positions selected from amino acid positions 3, 4, 6, 7, 9, 13, 15, 19, 22, 25, 31, 34, 36, 37, 38, 41, 42, 44, 45, 46, 55, 56, 69, 70, 72, 81, 86, 89, 92 or 97. In one embodiment the amino acid modifications are amino acid substitutions, and in one embodiment the substitutions are conservative amino acid substitutions. In one embodiment the peptide sequence differs from SEQ ID NO: 4 by 1, 2, 3, 4 or 5 amino acid modifications at positions selected from amino acid positions 3, 4, 6, 7, 9, 13, 15, 19, 22, 25, 31, 34, 36, 37, 38, 41, 42, 44, 45, 46, 55, 56, 69, 70, 72, 81, 86, 89, 92 or 97.

In one embodiment the method of neutralizing multi-drug resistant bacteria comprises contacting the bacteria with an interferon-inducible (ELR−) CXC chemokine at a concentration of about 1 to about 100 μg/ml, about 1 to about 75 μg/ml, about 1 to about 50 μg/ml, 1 to about 30 μg/ml, 1 to about 15 μg/ml, 2 to about 10 μg/ml, 4 to about 8 μg/ml, 6 to about 10 μg/ml or about 8 μg/ml.

Since interferons are known to induce expression of native CXCL9, CXCL10 and CXCL11, in one embodiment the method of treatment comprises the co-administration to a subject in need thereof one or more interferons, including for example interferon-alpha, interferon-beta and/or interferon-gamma as an adjuvant to promote production of native

CXCL9, CXCL10 and CXCL11 chemokines in vivo. Co-aministration can be accomplished by simultaneously administering the chemokine and the interferon, or the two active agents can be administered one after the other within 1, 2, 3, 4, 5, 6, 12, 24 or 48 hours of each other.

### Neutralizing Bacterial Spores

Spores are resistant to most agents that would normally kill the vegetative cells they formed from. Household cleaning products generally have no effect, nor do most alcohols, quaternary ammonium compounds or detergents. Currently, treatments are not available that are designed to decontaminate (e.g., neutralize and/or prevent the growth or germination of) spores on human skin or other human surfaces (e.g., lungs or hair). Thus, there is a need for compositions and methods that can neutralize and prevent the outgrowth of spores of pathogenic bacteria such as Bacillus anthracis. Such an agent would ideally be easily disseminated, not be harmful to human surfaces (e.g., skin or lungs) and would be capable of altering (e.g., inhibiting) spore germination and growth potential (e.g., thereby leaving the spores inert and non-infectious).

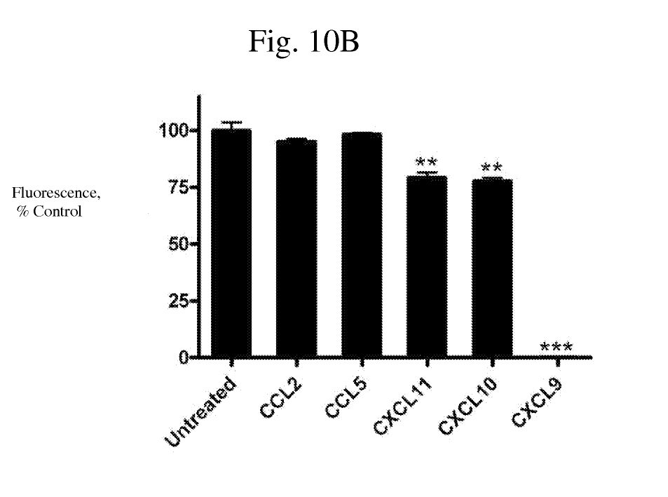

Surprisingly, applicants have discovered that interferon-inducible (ELR−) CXC chemokines are effective in neutralizing spores. Specifically, recombinant CXCL9, CXCL10, and CXCL11 exhibit direct inhibitory effects on spore germination and directly kill vegetative cells of B. anthracis (See FIGS. 10A & 10B). Furthermore, selective in vivo neutralization of CXCL9 or CXCL9/CXCL10, or CXCL9/CXCL10/CXCL11 rendered normally resistant C57BL/6 mice susceptible to pulmonary anthrax, whereas neutralization of their shared receptor, CXCR3 (i.e., the common receptor expressed on leukocytes recruited to the site of infection by CXCL9, CXCL10, CXCL11), had no impact on survival. These findings support the notion that interferon-inducible (ELR−) CXC chemokines have direct antimicrobial effects against B. anthracis in vitro and during in vivo infection.

In accordance with one embodiment a method of neutralizing spores, particularly of pathogenic bacteria such as B. anthracis and C. difficile is provided, wherein the method comprises contacting the spores with a composition comprising an interferon-inducible (ELR−) CXC chemokine In accordance with one embodiment the method comprises the steps of contacting the spores with an effective amount of a peptide selected from the group consisting of i) CXCL-9 (SEQ ID NO: 1), CXCL-10 (SEQ ID NO: 4) or CXCL 11 (SEQ ID NO: 7), ii) a peptide fragment of CXCL-9, CXCL-10 or CXCL 11, or a peptide having at least 90% amino acid sequence identity with i) or ii). In one embodiment the peptide comprises the sequence of SEQ ID NO: 3, SEQ ID NO: 6 and SEQ ID NO: 9, or a peptidomimetic derivative thereof. It is anticipated that compositions comprising the interferon-inducible (ELR−) CXC chemokines disclosed herein can be formulated for treating external surfaces (e g skin or hair) or can be formulated as pharmaceuticals for administration (e.g. inhaled formulations) to subjects to neutralize internalized (e.g., the lungs) spores in vivo.

In another embodiment the peptide comprises a sequence selected from the group consisting of i) SEQ ID NO: 3 or SEQ ID NO: 6 or a peptide having at least 95% amino acid sequence identity with SEQ ID NO: 3 or SEQ ID NO: 6 or SEQ ID NO: 9. In one embodiment the peptide comprises the sequence of SEQ ID NO: 15 or SEQ ID NO: 16. In a further embodiment the spores are contacted with a composition comprising an interferon-inducible (ELR−) CXC chemokine peptide, or peptidomimetic derivative thereof, wherein the peptide comprises a sequence selected from the group consisting of SEQ ID NO: 1, SEQ ID NO: 2 and SEQ ID NO: 4. In a further embodiment the composition comprises two or three peptides selected from the group consisting of SEQ ID NO: 1, SEQ ID NO: 2, and SEQ ID NO: 4. In one embodiment the composition comprises a peptide comprising the sequence of SEQ ID NO: 1, SEQ ID NO: 2 and SEQ ID NO: 4 or a sequence that is 95% identical in sequence with SEQ ID NO: 1, SEQ ID NO: 2 or SEQ ID NO: 4. In another embodiment the peptide comprises a peptide sequence that differs from SEQ ID NO: 4 by no more than 1, 2, 3, 4 or 5 amino acid modifications at positions selected from amino acid positions 3, 4, 6, 7, 9, 13, 15, 19, 22, 25, 31, 34, 36, 37, 38, 41, 42, 44, 45, 46, 55, 56, 69, 70, 72, 81, 86, 89, 92 or 97. In one embodiment the amino acid modifications are amino acid substitutions, and in a further embodiment the substitutions are conservative amino acid substitutions. In one embodiment the peptide sequence differs from SEQ ID NO: 4 by 1, 2, 3, 4 or 5 amino acid modifications at positions selected from amino acid positions 3, 4, 6, 7, 9, 13, 15, 19, 22, 25, 31, 34, 36, 37, 38, 41, 42, 44, 45, 46, 55, 56, 69, 70, 72, 81, 86, 89, 92 or 97.

The present invention is not limited by the type of bacterial spore neutralized. In some embodiments, the spore is a Bacillus spore, including for example a Bacillus anthracis spore. The Bacillus anthracis spore may be a naturally occurring spore or a genetically or mechanically engineered form. The spore may also be from an antibiotic resistant strain of B. anthracis (e.g., ciprofloxacin resistant). In some embodiments, the interferon-inducible (ELR−) CXC chemokine is administered to a subject under conditions such that spore germination or growth is prohibited and/or attenuated. In some embodiments, greater than 70%, 80%, or 90% of bacterial spores are neutralized (e.g., killed). In some embodiments, there is greater than 2 log (e.g., greater than 3 log, 4 log, 5 log, . . .) reduction in bacterial spore outgrowth. In some embodiments, reduction in spore outgrowth occurs within hours (e.g., with 1 hour (e.g., in 20-40 minutes or less), within 2 hours, within 3 hours, within 6 hours or within 12 hours). In some embodiments, neutralization of the spore (e.g., the inability of the spore to germinate) lasts for at least 3 days, at least 7 days, at least 14 days, at least 21 days, at least 28 days, or at least 56 days.

In one embodiment the method comprises contacting the spores with an interferon-inducible (ELR−) CXC chemokine at a concentration of about 1 to about 100 μg/ml, about 1 to about 75 μg/ml, about 1 to about 50 μg/ml, 1 to about 30 μg/ml, 1 to about 15 μg/ml, 2 to about 10 μg/ml, 4 to about 8 μg/ml, 6 to about 10 μg/ml or about 8 μg/ml.

Examples of spore-forming bacteria include the genera: Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Coxiella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, and Vulcanobacillus. However, the list of significant spore-forming pathogens is more limited.

Examples of the more problematic pathogens in the clinical setting include:

1) Bacillus anthracis: causative agent in pulmonary infection with dissemination and high mortality; cutaneous infection; gastrointestinal infections;

2) Bacillus cereus: causative agent in food poisoning from refried or re-heated rice, etc.;

3) Clostridium difficile: causative agent in diarrhea, megacolon, colonic perforation, etc.;

4) Clostridium botulinum (botulism);

5) Clostridium perfringens: causative agent in gastrointestinal infections and/or bloodstream infections;

6) Clostridium tetani (tetanus);

7) Clostridium sordellii: causative agent in gastrointestinal infections and/or bloodstream infections.

Of note, C. difficile is one of the most problematic spore-forming pathogens in hospitalized patients since it can cause severe diarrhea and even colonic rupture. Emergence of hypervirluent strains has occurred over the past few years with an observed higher mortality.

Surprisingly, applicants have found that the interferon-inducible (ELR−) CXC chemokines have activity in neutralizing spores under physiological conditions. In accordance with one embodiment a method is provided for neutralizing spores of a prokaryotic pathogenic organism. The method comprises contacting the spores with a composition comprising an interferon-inducible (ELR−) CXC chemokine. In accordance with one embodiment a method is provided for neutralizing spores from an organism selected from the group consisting of Bacillus anthracis, Bacillus cereus, Clostridium difficile, Clostridium botulinum, Clostridium perfringens, Clostridium tetani and Clostridium sordellii. In one embodiment the method comprises neutralizing spores from an organism selected from the group consisting of Bacillus anthracis and Clostridium difficile, and in one specific embodiment the method comprises neutralizing Bacillus anthracis spores.

In one embodiment the method of neutralizing bacterial spores comprises contacting the spores with a composition comprising an interferon-inducible (ELR−) CXC chemokine having a peptide sequence selected from the group consisting of SEQ ID NO: 3, SEQ ID NO: 6 and SEQ ID NO: 9, or a peptidomimetic derivative thereof. In another embodiment the spores are contacted with an interferon-inducible (ELR−) CXC chemokine having a peptide sequence selected from the group consisting of i) SEQ ID NO: 1, SEQ ID NO: 2, SEQ ID NO: 4 SEQ ID NO: 5, SEQ ID NO: 7 and SEQ ID NO: 8, ii) a peptide fragment of SEQ ID NO: 1, SEQ ID NO: 2, SEQ ID NO: 4, SEQ ID NO: 5, SEQ ID NO: 7 and SEQ ID NO: 8, or a peptide having at least 90% amino acid sequence identity with i) or ii). In a further embodiment the composition comprises an interferon-inducible (ELR−) CXC chemokine peptide, or peptidomimetic derivative thereof, wherein the peptide comprises a sequence selected from the group consisting of SEQ ID NO: 1, SEQ ID NO: 2, SEQ ID NO: 4 and SEQ ID NO: 7. In a further embodiment the composition comprises two or three peptides selected from the group consisting of SEQ ID NO: 1, SEQ ID NO: 2, SEQ ID NO: 4 and SEQ ID NO: 7. In one embodiment the composition comprises a peptide comprising the sequence of SEQ ID NO: 1, SEQ ID NO: 2, SEQ ID NO: 4 and SEQ ID NO: 7 or a sequence that is 95% identical in sequence with SEQ ID NO: 1, SEQ ID NO: 2, SEQ ID NO: 4 and SEQ ID NO: 7. In accordance with one embodiment the composition comprises the sequence of SEQ ID NO: 4 or a sequence that differs from SEQ ID NO: 4 by 1, 2, 3, 4, 5, 6, 7, 8, 9, or 10 amino acids at positions independently selected from positions 4, 7, 22, 25, 37, 44, 45, 72, 86, 91, 92, 97. In one embodiment the differences represent conservative amino acid substitutions. In one embodiment the peptide sequence differs from SEQ ID NO: 4 by 1, 2, 3, 4 or 5 amino acid modifications at positions selected from amino acid positions 3, 4, 6, 7, 9, 13, 15, 19, 22, 25, 31, 34, 36, 37, 38, 41, 42, 44, 45, 46, 55, 56, 69, 70, 72, 81, 86, 89, 92 or 97.

Since interferons are known to induce expression of native CXCL9, CXCL10 and CXCL11, in one embodiment the method of treating a patient who has come in contact with spores comprises the co-administration of one or more interferons, including for example interferon-alpha, interferon-beta and/or interferon-gamma as an adjuvant to promote production of native CXCL9, CXCL10 and CXCL11 chemokines in vivo. Co-aministration can be accomplished by simultaneously administering the chemokine and the interferon, or the two active agents can be administered one after the other within 1, 2, 3, 4, 5, 6, 12, 24 or 48 hours of each other.

In one embodiment the compositions further comprise additional anti-microbial agents including, for example, one or more antibiotics. In another embodiment the method comprises administering one or more interferon-inducible (ELR−) CXC chemokine peptides wherein the interferon-inducible (ELR−) CXC chemokine is linked, optionally via covalent bonding and optionally via a linker, to a conjugate moiety. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other.

The peptide can be linked to conjugate moieties via direct covalent linkage by reacting targeted amino acid residues of the peptide with an organic derivatizing agent that is capable of reacting with selected side chains or the N- or C-terminal residues of these targeted amino acids. Reactive groups on the peptide or conjugate include, e.g., an aldehyde, amino, ester, thiol, a-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, the conjugate moieties can be linked to the peptide indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier.

Exemplary conjugate moieties that can be linked to any of the glucagon peptides described herein include but are not limited to a heterologous peptide or polypeptide (including for example, a plasma protein), a targeting agent, an immunoglobulin or portion thereof (e.g. variable region, CDR, or Fc region), a diagnostic label such as a radioisotope, fluorophore or enzymatic label, a polymer including water soluble polymers, or other therapeutic or diagnostic agents.

In accordance with one embodiment a method of neutralizing spores is provided wherein a composition comprising an interferon-inducible (ELR−) CXC chemokine linked to a lipid vesicle is administered to a subject in need thereof. In one embodiment the interferon-inducible (ELR−) CXC chemokine is linked to the external surface of the lipid vesicle, and in one embodiment the interferon-inducible (ELR−) CXC chemokine is covalently bound to the lipids comprising the lipid vesicle. In an alternative embodiment the interferon-inducible (ELR−) CXC chemokine is entrapped within the lipid vesicle. In one embodiment the lipid vesicle is a liposome. In a further embodiment the composition comprises additional anti-microbial agents, including for example one or more antibiotics. It is anticipated that the administration of the interferon-inducible (ELR−) CXC chemokine will enhance the efficacy of the known anti-microbial agent. The known anti-microbial agents can be co-administered with the interferon-inducible (ELR−) CXC chemokine either in a single dosage form or the therapeutic agents can be administered sequentially, within 5, 10, 15, 30, 60, 120, 180, 440 minutes or 12, 24 or 48 hours, to one another. In one embodiment the interferon-inducible (ELR−) CXC chemokine is linked to a liposome, optionally with the known anti-microbial agents also linked to the same liposome.

In accordance with one embodiment the interferon-inducible (ELR−) CXC chemokine compositions disclosed herein are used to treat solid surfaces to neutralize spore contaminated surfaces. In one embodiment the compositions disclosed herein are used to decontaminate organic materials including food or the external surfaces of animals including human skin. In another embodiment the methods for neutralizing spores comprises administering a pharmaceutical composition comprising an interferon-inducible (ELR−) CXC chemokine to neutralize spores that have been internalized by a subject. In one embodiment the composition is formulated as an aerosol, mist, fine powder or other formulation known to those skilled in the art for administration to pulmonary system. In one embodiment the composition is formulated for oral delivery using formulations known to those skilled in the art for administration to the digestive tract.

### Methods of Identifying Antagonists and Inhibitors of FtsX

As used herein, an antagonist or inhibiting agent may comprise, without limitation, a drug, a small molecule, an antibody, an antigen binding portion thereof or a biosynthetic antibody binding site that binds a particular target protein; an antisense molecule that hybridizes in vivo to a nucleic acid encoding a target protein or a regulatory element associated therewith, or a ribozyme, aptamer, a phylomer or small molecule that binds to and/or inhibits a target protein, or that binds to and/or inhibits, reduces or otherwise modulates expression of nucleic acid encoding a target protein, including for example RNA interference (e.g., use of small interfering RNA (siRNA)).

This invention encompasses methods of screening compounds to identify those compounds that act as agonists (stimulate) or antagonists (inhibit) of the protein interactions and pathways described herein. Screening assays for antagonist compound candidates are designed to identify compounds that bind or complex with the peptides described herein, or otherwise interfere with the interaction of the peptides with other proteins. Such screening assays will include assays amenable to high-throughput screening of chemical libraries, making them particularly suitable for identifying small molecule drug candidates.

FtsX assays also include those described in detail herein, such as far-western, co-immunoprecipitation, immunoassays, immunocytochemical/immuno localization, interaction with FtsX protein, fertilization, contraception, and immunogenicity.

The assays can be performed in a variety of formats, including protein-protein binding assays, biochemical screening assays, high-throughput assays, immunoassays, and cell-based assays, which are well characterized in the art.

All assays for antagonists are common in that they call for contacting the compound or drug candidate with a peptide identified herein under conditions and for a time sufficient to allow these two components to interact.

In binding assays, the interaction is binding and the complex formed can be isolated or detected in the reaction mixture. In a particular embodiment, one of the peptides of the complexes described herein, or the test compound or drug candidate is immobilized on a solid phase, e.g., on a microtiter plate, by covalent or non-covalent attachments. Non-covalent attachment generally is accomplished by coating the solid surface with a solution of the peptide and drying. Alternatively, an immobilized antibody, e.g., a monoclonal antibody, specific for the peptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with, but does not bind to a particular peptide identified herein, its interaction with that peptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Complete kits for identifying protein-protein interactions between two specific proteins using the two-hybrid technique are available. This system can also be extended to map protein domains involved in specific protein interactions as well as to pinpoint amino acid residues that are crucial for these interactions.

Compounds that interfere with the interaction of a peptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the peptide may be added to a cell along with the compound to be screened for a particular activity and the ability of the compound to inhibit the activity of interest in the presence of the peptide indicates that the compound is an antagonist to the peptide. The peptide can be labeled, such as by radioactivity.

Other assays and libraries are encompassed within the invention, such as the use of phylomers® and reverse yeast two-hybrid assays (see Watt, 2006, Nature Biotechnology, 24:177; Watt, U.S. Pat. No. 6,994,982; Watt, U.S. Pat. Pub. No. 2005/0287580; Watt, U.S. Pat. No. 6,510,495; Barr et al., 2004, J. Biol. Chem., 279:41:43178-43189; the contents of each of these publications is hereby incorporated by reference herein in their entirety). Phylomers® are derived from sub domains of natural proteins, which makes them potentially more stable than conventional short random peptides. Phylomers® are sourced from biological genomes that are not human in origin. This feature significantly enhances the potency associated with Phylomers® against human protein targets. Phylogica's current Phylomer® library has a complexity of 50 million clones, which is comparable with the numerical complexity of random peptide or antibody Fab fragment libraries. An Interacting Peptide Library, consisting of 63 million peptides fused to the B42 activation domain, can be used to isolate peptides capable of binding to a target protein in a forward yeast two hybrid screen. The second is a Blocking Peptide Library made up of over 2 million peptides that can be used to screen for peptides capable of disrupting a specific protein interaction using the reverse two-hybrid system.

The Phylomer® library consists of protein fragments, which have been sourced from a diverse range of bacterial genomes. The libraries are highly enriched for stable subdomains (15-50 amino acids long). This technology can be integrated with high throughput screening techniques such as phage display and reverse yeast two-hybrid traps.

The present application discloses compositions and methods for inhibiting the proteins described herein, and those not disclosed which are known in the art are encompassed within the invention. For example, various modulators/effectors are known, e.g. antibodies, biologically active nucleic acids, such as antisense molecules, RNAi molecules, or ribozymes, aptamers, peptides or low-molecular weight organic compounds recognizing said polynucleotides or polypeptides.

The present application also encompasses pharmaceutical and therapeutic compositions comprising the compounds of the present invention.

The present application is also directed to pharmaceutical compositions comprising the peptides of the present invention. More particularly, such compounds can be formulated as pharmaceutical compositions using standard pharmaceutically acceptable carriers, fillers, solublizing agents and stabilizers known to those skilled in the art. The pharmaceutical compositions can be formulated to be administered using standard routes of administration including for example, oral, parenteral, topical and as an inhaled formulation, using standard formulations and techniques known to those skilled in the art.

Pharmaceutically-acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl)amines, tri(substituted alkyl)amines, alkenyl amines, dialkenyl amines, trialkenyl amines, substituted alkenyl amines, di(substituted alkenyl)amines, tri(substituted alkenyl)amines, cycloalkyl amines, di(cycloalkyl)amines, tri(cycloalkyl)amines, substituted cycloalkyl amines, disubstituted cycloalkyl amine, trisubstituted cycloalkyl amines, cycloalkenyl amines, di(cycloalkenyl)amines, tri(cycloalkenyl)amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri(iso-propyl)amine, tri(n-propyl)amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the practice of this invention, for example, carboxylic acid amides, including carboxamides, lower alkyl carboxamides, dialkyl carboxamides, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

Also encompassed by the present disclosures are antibodies raised against the proteins and peptides disclosed herein. The generation of polyclonal antibodies is accomplished by inoculating the desired animal with the antigen and isolating antibodies which specifically bind the antigen therefrom.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

The present disclosure also encompasses the use pharmaceutical compositions of an appropriate compound, homo log, fragment, analog, or derivative thereof to practice the methods of the invention, the composition comprising at least one appropriate compound, homo log, fragment, analog, or derivative thereof and a pharmaceutically-acceptable carrier.

The pharmaceutical compositions useful for practicing the invention may be administered to deliver a dose of between 1 ng/kg/day and 100 mg/kg/day. Pharmaceutical compositions that are useful in the methods of the invention may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the invention.

Compounds which are identified using any of the methods described herein may be formulated and administered to a mammal for treatment of the diseases disclosed herein are now described.

The invention encompasses the preparation and use of pharmaceutical compositions comprising a compound useful for treatment of the conditions, disorders, and diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations. The pharmaceutical compositions of the present invention can be processed into a tablet form, capsule form, or suspension that is suited for oral administration or can be reconstituted in an aqueous solvent (e.g., DI water or saline) for oral, IV, or inhalation (e.g., nebulizer) administration.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology. A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulo se. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may also be prepared, packaged, or sold in a formulation suitable for rectal administration, vaginal administration, parenteral administration

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parenterally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self propelling solvent/powder dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension (e.g., use of a nebulizer). Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1/1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the subject.

The compound may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type, and age of the subject, etc.

The invention also includes a kit comprising a compound of the invention and an instructional material which describes administering the composition to a cell or a tissue of a subject. In another embodiment, this kit comprises a (preferably sterile) solvent suitable for dissolving or suspending the composition of the invention prior to administering the compound to the subject. The invention also provides a kit for identifying a regulator of the target molecule of the invention.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

In one embodiment, the present method of immunization comprises the administration of a source of immunogenically active polypeptide fragments, said polypeptide fragments being selected from FtsX protein fragments and/or homologs thereof as defined herein before, said polypeptide fragments comprising dominant CTL and/or HTL epitopes and which fragments are between 18 and 45 amino acids in length. Peptides having a length between 18 and 45 amino acids have been observed to provide superior immunogenic properties as is described in WO 02/070006. In accordance with one embodiment an antigenic composition is provided comprising an isolated peptide having the sequence of SEQ ID NO: 10 or a contiguous 8 amino acid fragment of SEQ ID NO: 10. In accordance with one embodiment the antigenic composition further comprises an adjuvant.

Peptides may advantageously be chemically synthesized and may optionally be (partially) overlapping and/or may also be ligated to other molecules, peptides, or proteins. Peptides may also be fused to form synthetic proteins, as in Welters et al. (Vaccine. 2004 Dec. 2; 23(3):305-11). It may also be advantageous to add to the amino- or carboxy-terminus of the peptide chemical moieties or additional (modified or D-) amino acids in order to increase the stability and/or decrease the biodegradability of the peptide. To improve immunogenicity, immuno-stimulating moieties may be attached, e.g. by lipidation or glycosylation. To enhance the solubility of the peptide, addition of charged or polar amino acids may be used, in order to enhance solubility and increase stability in vivo.

For immunization purposes, the aforementioned immunogenic polypeptides of the invention may also be fused with proteins, such as, but not limited to, tetanus toxin/toxoid, diphtheria toxin/toxoid or other carrier molecules. The polypeptides according to the invention may also be advantageously fused to heatshock proteins, such as recombinant endogenous (murine) gp96 (GRP94) as a carrier for immunodominant peptides as described in (references: Rapp U K and Kaufmann S H, Int Immunol. 2004 April; 16(4):597-605; Zugel U, Infect Immun. 2001 June; 69(6):4164-7) or fusion proteins with Hsp70 (Triebel et al; WO9954464).

The individual amino acid residues of the present immunogenic (poly)peptides of the invention can be incorporated in the peptide by a peptide bond or peptide bond mimetic. A peptide bond mimetic of the invention includes peptide backbone modifications well known to those skilled in the art. Such modifications include modifications of the amide nitrogen, the alpha carbon, amide carbonyl, complete replacement of the amide bond, extensions, deletions, or backbone cross-links. See, generally, Spatola, Chemistry and Biochemistry of Amino Acids, Peptides and Proteins, Vol. VII (Weinstein ed., 1983). Several peptide backbone modifications are known and can be used in the practice of the invention.

Amino acid mimetics may also be incorporated in the polypeptides. An “amino acid mimetic” as used here is a moiety other than a naturally occurring amino acid that conformationally and functionally serves as a substitute for an amino acid in a polypeptide of the present invention. Such a moiety serves as a substitute for an amino acid residue if it does not interfere with the ability of the peptide to elicit an immune response against the native FtsX T cell epitopes. Amino acid mimetics may include non-protein amino acids. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the present invention are discussed by Morgan and Gainor, (1989) Ann. Repts. Med. Chem. 24:243-252.

In one embodiment, the present method comprises the administration of a composition comprising one or more of the present immunogenic polypeptides as defined herein above, and at least one excipient. Excipients are well known in the art of pharmacy and may for instance be found in textbooks such as Remington's pharmaceutical sciences, Mack Publishing, 1995.

The present method for immunization may further comprise the administration, and in one aspect, the co-administration, of at least one adjuvant. Adjuvants may comprise any adjuvant known in the art of vaccination and may be selected using textbooks like Current Protocols in Immunology, Wiley Interscience, 2004.

Adjuvants are herein intended to include any substance or compound that, when used, in combination with an antigen, to immunize a human or an animal, stimulates the immune system, thereby provoking, enhancing or facilitating the immune response against the antigen, preferably without generating a specific immune response to the adjuvant itself. In one aspect, adjuvants can enhance the immune response against a given antigen by at least a factor of 1.5, 2, 2.5, 5, 10, or 20, as compared to the immune response generated against the antigen under the same conditions but in the absence of the adjuvant. Tests for determining the statistical average enhancement of the immune response against a given antigen as produced by an adjuvant in a group of animals or humans over a corresponding control group are available in the art. The adjuvant preferably is capable of enhancing the immune response against at least two different antigens. The adjuvant of the invention will usually be a compound that is foreign to a human, thereby excluding immunostimulatory compounds that are endogenous to humans, such as e.g. interleukins, interferons, and other hormones.

A number of adjuvants are well known to one of ordinary skill in the art. Suitable adjuvants include, e.g., incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxy-phosphoryloxy)-ethylamine (CGP 19835A, referred to as MTP-PE), DDA (2 dimethyldioctadecylammonium bromide), polyIC, Poly-A-poly-U, RIBI™, GERBU™, Pam3™, Carbopol™, Specol™, Titermax™, tetanus toxoid, diphtheria toxoid, meningococcal outer membrane proteins, diphtheria protein CRM₁₉₇. Preferred adjuvants comprise a ligand that is recognized by a Toll-like-receptor (TLR) present on antigen presenting cells. Various ligands recognized by TLR's are known in the art and include e.g. lipopeptides (see, e.g., WO 04/110486), lipopolysaccharides, peptidoglycans, liopteichoic acids, lipoarabinomannans, lipoproteins (from mycoplasma or spirochetes), double-stranded RNA (poly I:C), unmethylated DNA, flagellin, CpG-containing DNA, and imidazoquinolines, as well derivatives of these ligands having chemical modifications.

The methods of immunization of the present application further encompass the administration, including the co-administration, of a CD40 binding molecule in order to enhance a CTL response and thereby enhance the therapeutic effects of the methods and compositions of the invention. The use of CD40 binding molecules is described in WO 99/61065, incorporated herein by reference. The CD40 binding molecule is preferably an antibody or fragment thereof or a CD40 Ligand or a variant thereof, and may be added separately or may be comprised within a composition according to the current invention. Such effective dosages will depend on a variety of factors including the condition and general state of health of the patient. Thus, dosage regimens can be determined and adjusted by trained medical personnel to provide the optimum therapeutic or prophylactic effect.

In the present method, the one or more immunogenic polypeptides are typically administered at a dosage of about 1 ug/kg patient body weight or more at least once. Often dosages are greater than 10 ug/kg. According to the present invention, the dosages preferably range from 1 ug/kg to 1 mg/kg.

In one embodiment typical dosage regimens comprise administering a dosage of 1-1000 ug/kg, more preferably 10-500 ug/kg, still more preferably 10-150 ug/kg, once, twice or three times a week for a period of one, two, three, four or five weeks. According to one embodiment, 10-100 ug/kg is administered once a week for a period of one or two weeks.

The present method, in one aspect, comprises administration of the present immunogenic polypeptides and compositions comprising them via the injection, transdermal, or oral route. In another, embodiment of the invention, the present method comprises vaginal administration of the present immunogenic polypeptides and compositions comprising them.

Another aspect of this disclosure relates to a pharmaceutical preparation comprising as the active ingredient the present source of a polypeptide as defined herein before. More particularly pharmaceutical preparation comprises as the active ingredient one or more of the aforementioned immunogenic polypeptides selected from the group of FtsX proteins, homologues thereof and fragments of said FtsX proteins and homologs thereof, or, alternatively, a gene therapy vector as defined herein above.

The present invention further provides a pharmaceutical preparation comprising one or more of the immunogenic polypeptides of the invention. The concentration of said polypeptide in the pharmaceutical composition can vary widely, i.e., from less than about 0.1% by weight, usually being at least about 1% by weight to as much as 20% by weight or more.

The composition may comprise a pharmaceutically acceptable carrier in addition to the active ingredient. The pharmaceutical carrier can be any compatible, non-toxic substance suitable to deliver the immunogenic polypeptides or gene therapy vectors to the patient. For polypeptides, sterile water, alcohol, fats, waxes, and inert solids may be used as the carrier. Pharmaceutically acceptable adjuvants, buffering agents, dispersing agents, and the like, may also be incorporated into the pharmaceutical compositions.

In one embodiment, the present pharmaceutical composition comprises an adjuvant, as defined in more detail herein before. Adjuvants useful for incorporation in the present composition are preferably selected from the group of ligands that are recognized by a Toll-like-receptor (TLR) present on antigen presenting cells, including lipopeptides, lipopolysaccharides, peptidoglycans, liopteichoic acids, lipoarabinomannans, lipoproteins (from mycoplasma or spirochetes), double-stranded RNA (poly I:C), unmethylated DNA, flagellin, CpG-containing DNA, and imidazoquinolines, as well derivatives of these ligands having chemical modifications. The routineer will be able to determine the exact amounts of anyone of these adjuvants to be incorporated in the present pharmaceutical preparations in order to render them sufficiently immunogenic. According to another preferred embodiment, the present pharmaceutical preparation may comprise one or more additional ingredients that are used to enhance CTL immunity as explained herein before. According to a particularly preferred embodiment, the present pharmaceutical preparation comprises a CD40 binding molecule.

Methods of producing pharmaceutical compositions comprising polypeptides are described in U.S. Pat. Nos. 5,789,543 and 6,207,718. The preferred form depends on the intended mode of administration and therapeutic application.

In one embodiment, the present immunogenic proteins or polypeptides are administered by injection. The parenteral route for administration of the polypeptide is in accordance with known methods, e.g. injection or infusion by intravenous, intraperitoneal, intramuscular, intra-arterial, subcutaneous, or intralesional routes. The protein or polypeptide may be administered continuously by infusion or by bolus injection. A typical composition for intravenous infusion could be made up to contain 10 to 50 ml of sterile 0.9% NaCl or 5% glucose optionally supplemented with a 20% albumin solution and between 10 ug and 50 mg, preferably between 50 ug and 10 mg, of the polypeptide. A typical pharmaceutical composition for intramuscular injection would be made up to contain, for example, 1-10 ml of sterile buffered water and between 10 ug and 50 mg, preferably between 50 ug and 10 mg, of the polypeptide of the present invention. Methods for preparing parenterally administrable compositions are well known in the art and described in more detail in various sources, including, for example, Remington's Pharmaceutical Science (15th ed., Mack Publishing, Easton, Pa., 1980) (incorporated by reference in its entirety for all purposes).

For convenience, immune responses are often described in the present invention as being either “primary” or “secondary” immune responses. A primary immune response, which is also described as a “protective” immune response, refers to an immune response produced in an individual as a result of some initial exposure (e.g., the initial “immunization”) to a particular antigen. Such an immunization can occur, for example, as the result of some natural exposure to the antigen (for example, from initial infection by some pathogen that exhibits or presents the antigen). Alternatively, the immunization can occur because of vaccinating the individual with a vaccine containing the antigen. For example, the vaccine can be a vaccine comprising one or more antigenic epitopes or fragments of FtsX.

The vaccine can also be modified to express other immune activators such as IL2, and co-stimulatory molecules, among others.

Another type of vaccine that can be combined with antibodies to an antigen is a vaccine prepared from a cell lysate of interest, in conjunction with an immunological adjuvant, or a mixture of lysates from cells of interest plus DETOX™ immunological adjuvant. Vaccine treatment can be boosted with anti-antigen antibodies, with or without additional chemotherapeutic treatment.

When used in vivo for therapy, the antibodies of the subject invention are administered to the subject in therapeutically effective amounts (i.e., amounts that have desired therapeutic effect). They will normally be administered parenterally. The dose and dosage regimen will depend upon the degree of the infection, the characteristics of the particular antibody or immunotoxin used, e.g., its therapeutic index, the patient, and the patient's history. Advantageously the antibody or immunotoxin is administered continuously over a period of 1-2 weeks or longer as indicated or needed. Optionally, the administration is made during the course of adjunct therapy such as antimicrobial treatment, or administration of tumor necrosis factor, interferon, or other cytoprotective or immunomodulatory agent.

For parenteral administration, the antibodies will be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1.0 mg/ml to about 10 mg/ml.

Use of IgM antibodies can be preferred for certain applications; however, IgG molecules by being smaller can be more able than IgM molecules to localize to certain types of infected cells.

There is evidence that complement activation in vivo leads to a variety of biological effects, including the induction of an inflammatory response and the activation of macrophages (Unanue and Benecerraf, Textbook of Immunology, 2nd Edition, Williams & Wilkins, p. 218 (1984)). The increased vasodilation accompanying inflammation can increase the ability of various agents to localize. Therefore, antigen-antibody combinations of the type specified by this invention can be used in many ways. Additionally, purified antigens (Hakomori, Ann. Rev. Immunol. 2:103, 1984) or anti-idiotypic antibodies (Nepom et al., Proc. Natl. Acad. Sci. USA 81: 2864, 1985; Koprowski et al., Proc. Natl. Acad. Sci. USA 81: 216, 1984) relating to such antigens could be used to induce an active immune response in human patients.

The antibody compositions used are formulated and dosages established in a fashion consistent with good medical practice taking into account the condition or disorder to be treated, the condition of the individual patient, the site of delivery of the composition, the method of administration, and other factors known to practitioners. The antibody compositions are prepared for administration according to the description of preparation of polypeptides for administration, infra.

As is well understood in the art, biospecific capture reagents include antibodies, binding fragments of antibodies which bind to activated integrin receptors on metastatic cells (e.g., single chain antibodies, Fab′ fragments, F(ab)′2 fragments, and scFv proteins and affibodies (Affibody, Teknikringen 30, floor 6, Box 700 04, Stockholm SE-10044, Sweden; See U.S. Pat. No. 5,831,012, incorporated herein by reference in its entirety and for all purposes)). Depending on intended use, they also can include receptors and other proteins that specifically bind another biomolecule.

The hybrid antibodies and hybrid antibody fragments include complete antibody molecules having full length heavy and light chains, or any fragment thereof, such as Fab, Fab′, F(ab′)2, Fd, scFv, antibody light chains and antibody heavy chains. Chimeric antibodies which have variable regions as described herein and constant regions from various species are also suitable. See for example, U.S. Application No. 20030022244.

Initially, a predetermined target object is chosen to which an antibody can be raised. Techniques for generating monoclonal antibodies directed to target objects are well known to those skilled in the art. Examples of such techniques include, but are not limited to, those involving display libraries, xeno or humab mice, hybridomas, and the like. Target objects include any substance which is capable of exhibiting antigenicity and are usually proteins or protein polysaccharides. Examples include receptors, enzymes, hormones, growth factors, peptides and the like. It should be understood that not only are naturally occurring antibodies suitable for use in accordance with the present disclosure, but engineered antibodies and antibody fragments which are directed to a predetermined object are also suitable.

The present disclosure also encompasses a kit comprising the compounds of the invention or assay components of the invention and an instructional material that describes administration of the compounds or the assay. In another embodiment, this kit comprises a (preferably sterile) solvent suitable for dissolving or suspending the composition of the invention prior to administering the compound to the mammal.

Various aspects and embodiments of the invention are described in further detail below.

### EXAMPLE 1

Chemokines CXCL9, CXCL10, and CXCL11 Antimicrobial Activity

We tested whether human CXCL9, CXCL10, and CXCL11 exhibited antimicrobial activity against B. anthracis. These interferon-inducible (ELR−) CXC chemokines exhibited not only antimicrobial activity against the vegetative form of the organism, but also the spore form such that spore germination was blocked or reduced. An effect on spores is unprecedented, even for any of the traditional antibiotics. We found a hierarchy of activity with human CXCL10>CXCL9>CXCL11 in their ability to kill bacilli and block spore germination. We also tested the effects of recombinant murine CXCL9, CXCL10, and CXCL11 and found similar effects but with a different hierarchy of activity: CXCL9>CXCL10>CXCL11 (of note, human CXCL10 and murine CXCL9 exhibit very similar antimicrobial and anti-spore effects at the same concentrations).

Unless otherwise stated, recombinant human Interferon-inducible (ELR−) CXC chemokines were used for the in vitro studies herein. As controls, we used two recombinant human or mouse C-C family chemokines (CCL2 and CCL5) that have a similar molecular mass and charge (isoelectric point) as CXCL9, CXCL10, and CXCL11, but had no antimicrobial activity against B. anthracis spores or bacilli. The initial concentration of the interferon-inducible (ELR−) CXC chemokines used in our in vitro studies was 48 ug/ml. The 50% effective concentration (EC50) is 4-6 ug/ml for human CXCL10 or murine CXCL9, based on concentration curves using 0-72 ug/ml of interferon-inducible (ELR−) CXC chemokine. Although these concentrations may seem high based on the recognized potency of these interferon-inducible (ELR−) CXC chemokines as chemoattractants for recruitment of cells from distant locations, the local concentrations generated by and around cells in the lungs are likely higher. In this vein, these concentrations are commensurate with concentrations recovered from nasal secretions and stimulated by interferon-y in cell culture.

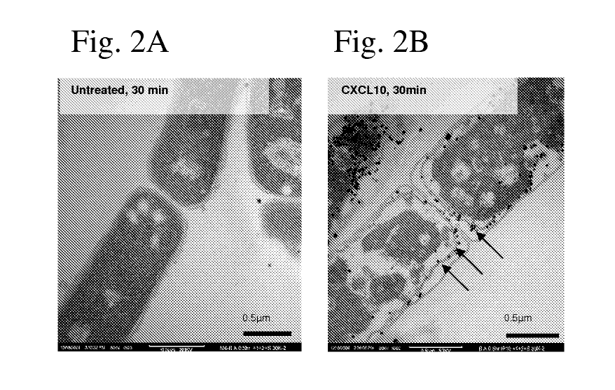

Immunogold electron microscopy (EM) studies of spores treated with CXCL10 demonstrated that CXCL10 localized internal to (not outside) the protective exosporium layer of the spores. In vegetative cells, CXCL10 localized primarily to the cell membrane (FIG. 2). These findings suggest that interaction of these interferon-inducible (ELR−) CXC chemokines with B. anthracis spores and vegetative cells is not simply due to a charge-charge interaction with random distribution at the outer surface of the organisms. Preliminary studies performed with stationary phase vegetative bacilli revealed >10-fold more potent CXCL10 killing effect with an EC50 value of 0.33 μg/m1 (FIG. 11C), compared to our previously reported EC50 value of 4-6 μg/ml for CXCL10 against exponential phase organisms.

These findings suggest that the interaction of these interferon-inducible (ELR−) CXC chemokines with B. anthracis spores and bacilli is not simply due to a charge-charge interaction with random distribution at the outer surface of the organisms.

### EXAMPLE 2

In vivo Activity of CXCL9, CXCL10, and CXCL11

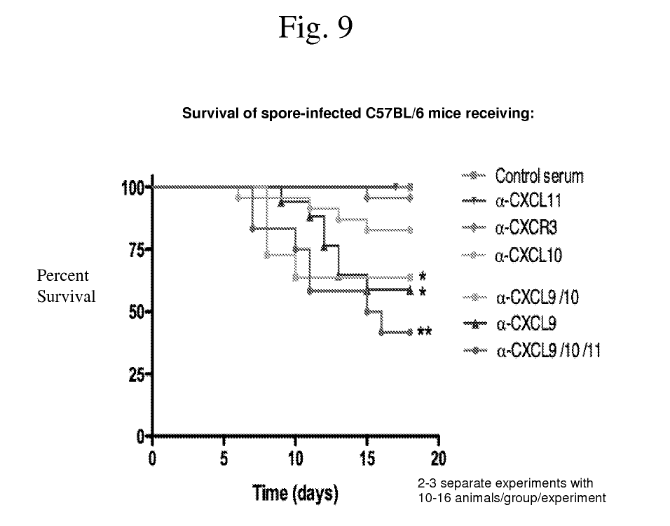

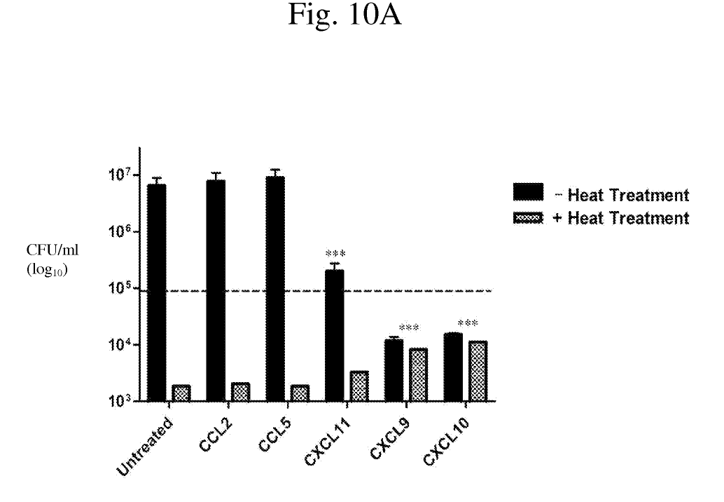

To test the biological relevance of CXCL9, CXCL10, and CXCL11 in vivo, we initially conducted a study comparing susceptible A/J and resistant C57BL/6 mice inoculated with B. anthracis Sterne strain spores that luminesce when undergoing germination. Using an in vivo Imaging System (IVIS), spore germination was monitored over time after intranasal inoculation of spores; little to no spore germination occurred in the lungs of the resistant C57BL/6 mice while highly detectable levels of germination were detected in the lungs of the A/J mice. Measurement of CXCL9, CXCL10, and CXCL11 levels in lung homogenates from these animals revealed that C57BL/6 mice had significantly higher levels of CXCL9 and CXCL10 after spore inoculation than did A/J mice. In vivo neutralization studies to further test the biological significance of these interferon-inducible (ELR−) CXC chemokines revealed (FIG. 9) that antibody neutralization of CXCL9, CXCL9/ CXCL10, or CXCL9/CXCL10/CXCL11, but not CXCR3, rendered the C57BL/6 mice significantly more susceptible to B. anthracis Sterne strain infection than the serum control-treated animals. We obtained similar data using CXCR3 knockout mice as well. These data support that there is a direct antimicrobial effect of these interferon-inducible (ELR−) CXC chemokines in vivo as well as in vitro. Furthermore the antimicrobial activity of both human and murine CXCL9, CXCL10, CXCL11 has been established using physiological salt concentrations against B. anthracis Sterne strain spores and bacilli.

Our observation that these interferon-inducible (ELR−) CXC chemokines have antimicrobial activities against spores and bacilli is strikingly novel and opens up an exciting avenue of research for studying host interferon-inducible (ELR−) CXC chemokines as direct antimicrobial agents and for developing novel therapeutic strategies using these interferon-inducible (ELR−) CXC chemokines. To begin to determine the mechanism of action of these interferon-inducible (ELR−) CXC chemokines against this bacterial pathogen, we have used a highly innovative genetic screening approach and identified a putative bacterial target of CXCL10; this target is annotated in the B. anthracis genome as FtsX, the permease component of an ATP-binding cassette (ABC) transporter that is widely conserved among Gram-positive and Gram-negative bacterial species. The identification of a putative bacterial target opens up exciting possibilities for novel therapeutic targets using the interferon-inducible (ELR−) CXC chemokines

### EXAMPLE 3

Determination that FtsX is the Target for CXCL9, CXCL10, and CXCL11

FtsX Sequence Information

Protein accession number: YP_{—}031272.1

297 amino acids:

mkaktlsrhl regvknlsrn gwmtfasysa vtvtlllvgv fltaimnmnh fatkveqdve irvhidpaak eadqkkledd mskiakvesi kysskeeelk rlikslgdsg ktfelfeqdnplknvfvvka keptdtatia kkiekmqfvs nvqygkgqve rlfdtvktgr nigivliagllftamflisn tikitiyars teieimklvg atnwfirwpf lleglflgvl gsiipiglil vtynslqgmf neklggtife llpyspfvfq lagllvliga ligmwgsvms irrflkv (SEQ ID NO: 10)

Other Relevant Information:

1: BAS5033 cell division ABC transporter, permease protein FtsX [Bacillus anthracis str. Sterne]:

GeneID: 2852087; Gene symbol-BAS5033; Gene description-cell division ABC transporter, permease protein FtsX; Locus tag-BAS5033; Gene type- protein coding; Organism-Bacillus anthracis str. Sterne (strain: Sterne); Lineage-Bacteria; Firmicutes; Bacillales; Bacillaceae; Bacillus; Bacillus cereus group;

NCBI Reference Sequence-NC_{—}005945.1; Bacillus anthracis str. Sterne, complete genome; >gi|49183039:c4906725-4905832 Bacillus anthracis str. Sterne, complete genome:

(SEQ ID NO: 11)
ATGAAGGCTAAGACCCTTAGTCGACATTTGCGAGAAGGTGTGAAAAATCT
ATCCCGTAACGGATGGATGACGTTTGCTTCTGTTAGTGCAGTAACAGTTA
CACTATTACTTGTAGGTGTCTTTTTAACAGCGATTATGAATATGAACCAT
TTTGCGACGAAAGTAGAGCAAGATGTTGAGATTCGTGTACACATTGATCC
AGCAGCAAAAGAAGCTGATCAAAAGAAATTAGAAGATGATATGAGTAAGA
TTGCAAAAGTAGAATCTATTAAATATTCTTCTAAAGAAGAAGAGTTAAAA
CGTTTAATTAAAAGCTTAGGCGATAGCGGAAAGACGTTTGAGTTATTTGA
ACAAGATAACCCACTGAAAAACGTGTTCGTTGTAAAAGCGAAAGAACCAA
CAGATACAGCAACAATTGCGAAAAAGATTGAAAAAATGCAGTTTGTAAGT
AATGTTCAGTACGGAAAAGGGCAAGTTGAACGATTATTTGATACTGTAAA
AACTGGTCGTAACATTGGTATTGTGTTAATTGCTGGTCTTTTATTCACAG
CGATGTTCTTAATCTCTAACACAATTAAAATTACAATTTATGCTCGTAGT
ACAGAAATCGAAATTATGAAACTTGTAGGTGCAACAAACTGGTTTATTCG
TTGGCCGTTCTTGTTAGAGGGATTATTCCTAGGAGTATTAGGATCAATTA
TTCCAATTGGCTTAATTCTTGTTACGTATAATTCACTACAAGGTATGTTT
AACGAAAAACTTGGCGGAACAATTTTCGAACTTCTACCATATAGTCCGTT
CGTATTCCAATTAGCTGGTTTACTAGTATTAATTGGGGCTTTAATCGGTA
TGTGGGGAAGCGTAATGTCAATTCGTCGTTTCTTAAAAGTATAA

The interferon-inducible (ELR−) CXC chemokines, CXCL9, CXCL10 and CXCL11, are important components of host defense in a variety of infections. We now have evidence that interferon-inducible (ELR−) CXC chemokines have direct in vitro antimicrobial activity against B. anthracis spores and bacilli.

1) Human and murine CXCL9, CXCL10, CXCL11 have direct antimicrobial activity at physiological salt concentrations against B. anthracis Sterne strain spores and bacilli in vitro, albeit with different hierarchies of activity: humanCXCL10>humanCXCL9>humanCXCLl11versus murineCXCL9>murineCXCL10>murineCXCL11 (notably, humanCXCL10 and murineCXCL9 have equivalent in vitro antimicrobial effects).

2) By immunogold EM imaging, CXCL10 localizes to spore structures within and internal to the exosporium, namely, to the spore coat and spore cortex; in vegetative cells, CXCL10 localizes to the cell membrane (FIG. 2).

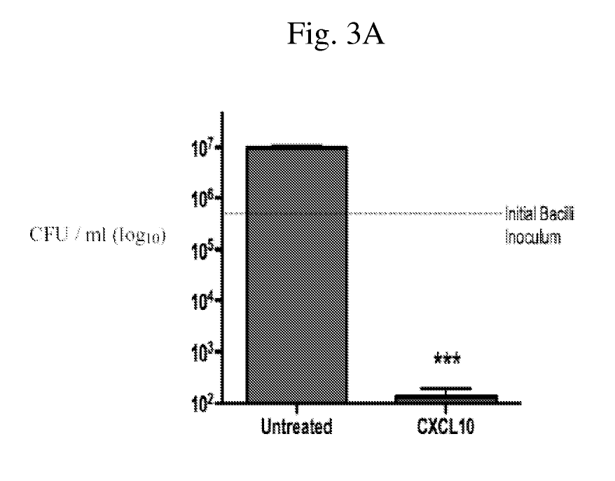

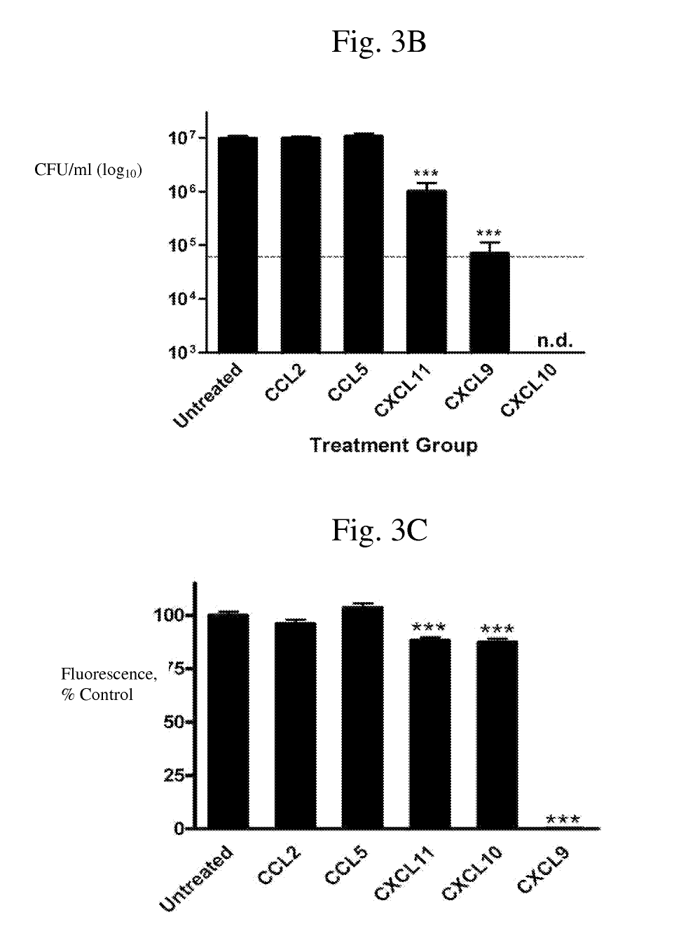

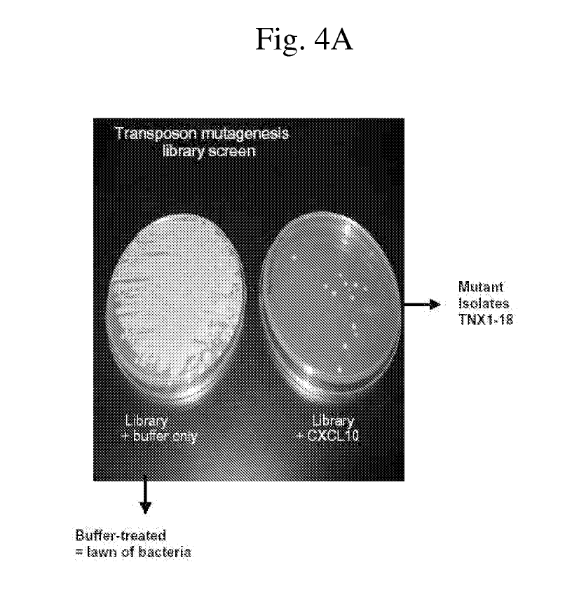

3) CXCL10 exhibits direct antimicrobial activity against spores and encapsulated cells of B. anthracis Ames strain (FIG. 3A) and against B. anthracis Sterne strain (FIGS. 3B & 3C). These data indicate that the CXC chemokines have antimicrobial effects against both unencapsulated and encapsulated strains of B. anthracis and support the use of Sterne strain as a model organism for the proposed studies.

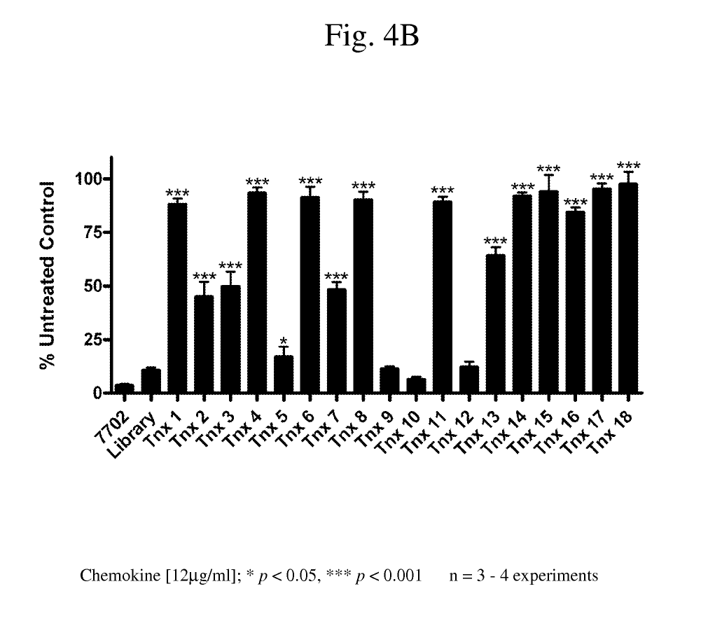

4) Initial screen of a B. anthracis Sterne strain transposon mutagenesis library using CXCL10 yielded a number of resistant bacterial isolates that are clones—the disrupted gene is annotated as ftsX and encodes the permease component of a prokaryotic ABC transporter (FIG. 4).

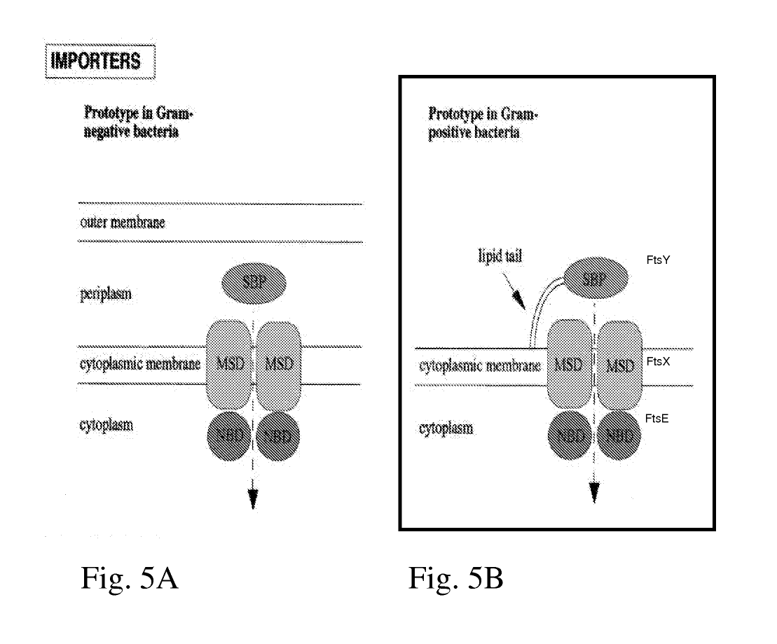

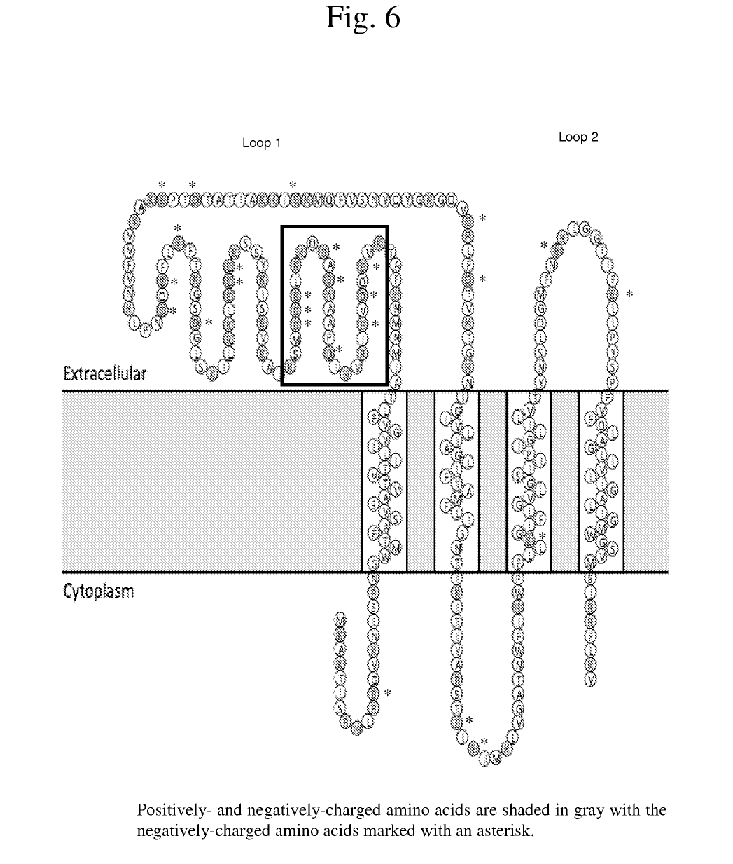

Identification of a putative bacterial target of CXCL10. We used an innovative genetic approach to identify a chemokine target in B. anthracis bacilli. This approach entailed use of a mariner-based transposon mutagenesis library adapted for B. anthracis Sterne strain from Listeria monocytogenes and developed by investigators at University of California, Berkeley (Zemansky, (2009) J. Bacteriol. 191:3950-3964). The transposon randomly inserts into the chromosomal and plasmid DNA and is designed to allow sequencing of regions flanking the transposon insertion, thus enabling rapid identification of the disrupted gene. We screened the B. anthracis transposon mutagenesis library for mutants that were resistant (or less susceptible) to CXCL10 and identified eighteen bacterial isolates (TNX1-18) resistant to CXCL10 in two independent screens; 10 of these 18 isolates were confirmed to be resistant to CXCL10 using an Alamar Blue viability assay (FIG. 4). In multiple isolates, the disrupted gene was identified by PCR and DNA sequencing as BAS5033, annotated as ftsX. This gene has a high degree of homology to the gene that encodes the Bacillus subtilis FtsX, an integral membrane protein component of an ABC transporter (FIG. 5) that functions by importing signals involved in the initiation of sporulation. This finding raises intriguing questions about whether the B. anthracis homologue of FtsX plays a role in transporting components/nutrients related to the maintenance of viability and is a direct (or indirect) target of CXCL10, or alternatively is involved in the uptake of CXCL10 into the organism. A predicted topology of the B. anthracis FtsX is shown in FIG. 6.

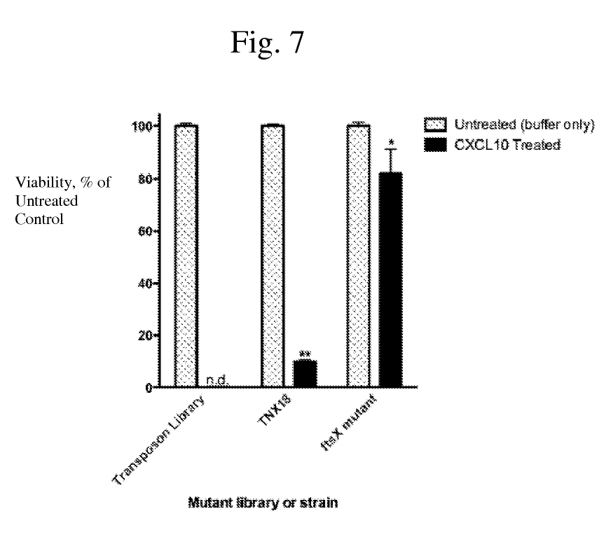

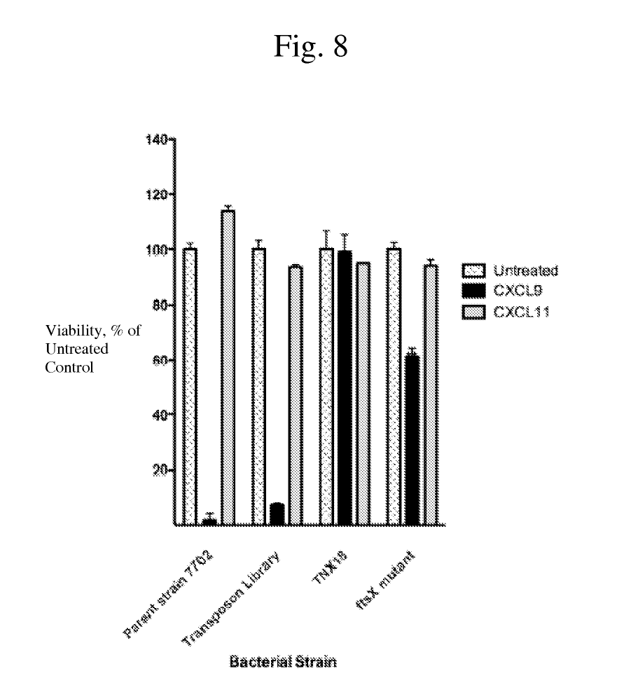

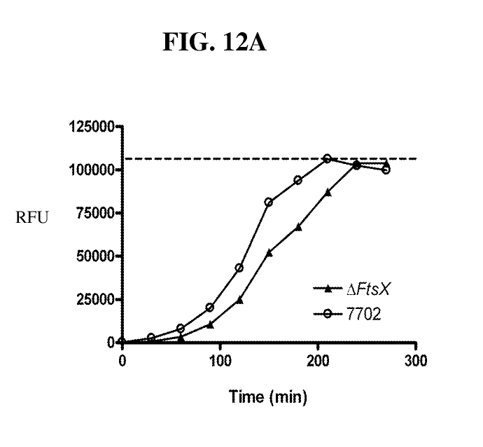

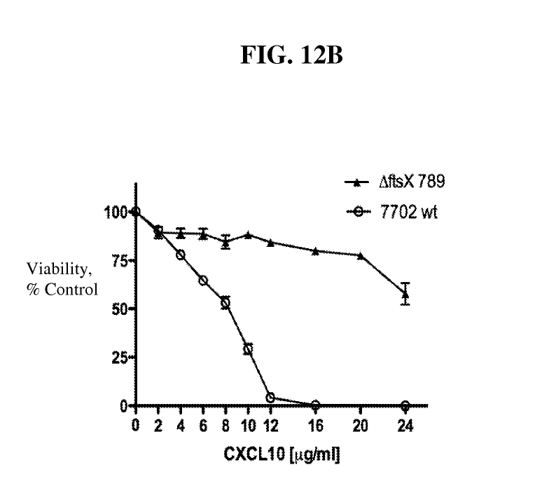

We successfully created a knockout mutant of ftsX by bacteriophage transduction (designated as the “ftsX mutant” or ΔftsX mutant or ΔftsX) using published protocols (43) and confirmed resistance of this mutant to CXCL10 (FIG. 7 and FIG. 12B). Furthermore, we have found that this mutant strain is also resistant to CXCL9 and CXCL11 (FIG. 8), which supports our hypothesis that CXCL9, CXCL10, and CXCL11 have a common target in vegetative bacteria.

Generation of a clean deletion mutant (designated “ΔftsX”) will allow for gene complementation analysis to verify that the original (susceptible) phenotype is restored. Once validated, the ΔftsX strain will be tested in vitro for its resistance to various concentrations CXCL9, CXCL10, and/or CXCL11. EM will be used to assess the structural integrity of the 4ftsX bacilli treated with CXCL9, CXCL10, or CXCL11, and immunogold EM will be used to assess interferon-inducible (ELR−) CXC chemokine localization in ΔftsX bacilli compared to that in wildtype Sterne 7702 bacilli.

Co-localization studies. To address the hypothesis that CXCL10 interacts directly with FtsX rather than indirectly (by affecting a molecule that interacts with FtsX), we will take the following approach. Because there are no FtsX-specific antibodies available at this time, we will generate a tagged version of the B. anthracis permease. In collaboration with Dr. Stibitz, we plan to generate an FtsX-GFP fusion protein with the GFP located at the carboxyl terminal end of FtsX using allelic exchange (see reference 61) methodology previously employed in B. anthracis. Our choice of GFP is based on published studies of GFP fusion proteins in B. anthracis and successful expression and use of FtsX-GFP fusion proteins generated in B. subtilis and other bacterial species. Advantages to using a GFP tag are that we will be able to monitor the location and expression of FtsX at various stages of growth during the experiments, which may prove important if B. anthracis is killed by CXCL10 by, for example, disruption of cell division by inhibiting septal ring formation (FtsX localizes to septal rings in B. subtilis (34a)). Importantly, the tag introduced into FtsX must not interfere with the function of the transporter. Since the substrate transported by FtsX is unknown, we will test for potential disruption of FtsX function by assessing bacterial growth in medium alone (no chemokine), monitor kinetics of cell division in log phase, monitor formation of septal rings using a GFP tagged version of FtsX under fluorescence microscopy as per published protocols (see references 28, 41), and assess the ability of bacilli to form spores (since FtsX in B. subtilis is thought to play a critical role in sporulation). We will perform EM to assess the structural integrity of the bacteria expressing untagged versus tagged FtsX at various stages of vegetative growth and sporulation.

Once a B. anthracis strain with a GFP-tagged FtsX is created and tested, we will examine the susceptibility of the organism to CXCL10 to ensure that addition of the tag has not altered the antimicrobial effect of CXCL10 against the bacilli. We will then perform co-localization studies with CXCL10 using immunofluorescence/confocal microscopy to study the interaction of CXCL10 with FtsX at various time points. B. anthracis cells that produce FtsX-GFP will be fixed and permeabilized using standard protocols familiar to the PI (58) and tested to ensure that the fixation process did not reduce or quench the GFP signal. If this does occur, an alternative approach would be to add anti-GFP antibodies after the bacteria are fixed and permeabilized followed by fluorescent-labeled secondary antibody. Anti-CXCL10 antibodies will be used followed by a (red) fluorescent-labeled secondary antibody.

Immunofluorescence/confocal microscopy will be performed to determine the individual locations of the CXCL10 and FtsX in the bacteria and if there is co-localization by appearance of a yellow signal (overlap of red and green signals). Similar studies will be performed using CXCL9 and CXCL11.

Site-directed mutagenesis studies. Without wishing to be bound by any particular theory, we hypothesize that CXCL10 (as well as CXCL9 and CXCL11) interacts with the predicted extracellular portions of FtsX, designated as Loops 1 and 2 in FIG. 6. To assess which portions of FtsX may interact with CXCL10, we will use allelic exchange to create deletion mutants of segments of Loop 1 and Loop 2. Depending on results obtained with mutants of Loop 1 and Loop 2, additional mutants of Loop 3 and 4 (predicted intracellular loops) will be generated. If deletion of a segment of FtsX abrogates the CXCL10 effect on the bacilli, we will narrow our studies to focus on key amino acids responsible for the interaction and/or effect of CXCL10; to do this, we will perform site-directed mutagenesis with substitution of neutral amino acids (alanine) for select amino acids in the portion of FtsX that may be responsible for the interaction or effect of CXCL10. We will initially target negatively charged amino acids that are clustered together since the net charge distribution is likely to play an important role in the interaction with CXCL10, which has a positively charged carboxyl terminus. The ability of the site-directed mutagenesis to disrupt interactions between the interferon-inducible (ELR−) CXC chemokine and FtsX will be assessed by in vitro susceptibility testing of the mutant bacterial strain to the interferon-inducible (ELR−) CXC chemokine Further, using a GFP-tagged version of FtsX, we will perform: 1) co-localization studies with immunofluorescence microscopy; and 2) immunoprecipitation coupled with Western blot analyses to determine if the mutated FtsX can be co-precipitated with antibodies to the specific interferon-inducible (ELR−) CXC chemokine

Expected results and interpretations. We expect that FtsX is the target for CXCL9, CXCL10, and CXCL11. Furthermore, we anticipate that the interaction between chemokine and FtsX is a direct interaction at the extracellular portion of the permease at a location where there is a net negative charge distribution. We anticipate that co-localization immunofluorescence experiments will reveal that the proteins interact at the cell membrane. Further, we predict that performing site-directed mutagenesis of select extracellular portions and then select (negatively charged) amino acids will abrogate the interaction and the antimicrobial effect of the interferon-inducible (ELR−) CXC chemokine against the bacilli.

Future extensions of these studies. In the studies described above, our focus has been on studying the interaction of the interferon-inducible (ELR−) CXC chemokines with vegetative bacilli with primary attention to the role of FtsX as a putative target. This represents the first description of the direct antimicrobial activity of interferon-inducible (ELR−) CXC chemokines against spores. This finding opens up the possibility of developing anti-spore therapeutics that could be used as an adjunct to conventional antimicrobials that only act against the vegetative form of the organism. Based on the structural differences and metabolic activities of these two different forms of the same organism, we hypothesize a priori that the interferon-inducible (ELR−) CXC chemokine targets and mechanisms of action differ between spores and bacilli. In fact, preliminary testing of spores produced by the ftsX mutant strain were not resistant to CXCL10. We propose as a future extension of our studies to pursue identification of spore targets of the interferon-inducible (ELR−) CXC chemokines The approach will entail screening of B. anthracis spores derived from sporulation of the vegetative transposon mutagenesis library.

Timing of chemokine-spore interaction will require careful monitoring since any spores resistant to the chemokine will germinate under germination-permissive conditions. Since there is no assurance that the resultant bacilli will be resistant to the chemokine present in the medium, the bacilli will likely be killed and not be identified as an interferon-inducible (ELR−) CXC chemokine resistant spore isolate. An alternative approach will be to incubate spores with the interferon-inducible (ELR−) CXC chemokine under germination non-permissive conditions (i.e., water or medium with no serum) for the minimal time of 60 minutes required for interferon-inducible (ELR−) CXC chemokine exposure to elicit an antimicrobial effect on the spores, based on washout experiments and then place the spores in germination permissive medium without interferon-inducible (ELR−) CXC chemokine. Bacilli derived from the chemokine-resistant spores will be isolated for further analysis and identification of mutant gene(s).

### EXAMPLE 4

Utilizing the interferon-inducible interferon-inducible (ELR−) CXC chemokines to elicit a protective effect in vivo against pulmonary anthrax infection in a mouse model.

The data described herein support the notion that the interferon-inducible interferon-inducible (ELR−) CXC chemokines play a direct and critical role in protecting the host against pulmonary anthrax. In addition to the in vitro data already presented in Examples 1-3, in vivo data provide further support as follows:

1) IFN-γ, CXCL9, CXCL10, CXCL11 are markedly induced and expressed early in the lungs of C57BL/6 mice, which are highly resistant to inhalational spore challenge.

2) CXCL10−/− mice have significantly higher numbers (CFUs) of B. anthracis spores and vegetative bacilli after spore challenge than do the wildtype parent C57BL/6 mice.

3) Antibody neutralization of CXCL9, CXCL9/CXCL10, CXCL9/CXCL10/CXCL11 significantly increased the susceptibility of C57BL/6 mice to anthrax infection but neutralization of the chemokine receptor (CXCR3) had no significant effect on C57BL/6 susceptibility to inhalational anthrax (FIG. 9).

Neutralization of CXCL9, CXCL9/CXCL10, or CXCL9/CXCL10/CXCL11 but not CXCR3 renders C57BL/6 mice susceptible to B. anthracis spore challenge. To assess the biological role of CXCL9, CXCL10, CXCL11, or their shared CXCR3 receptor (which is expressed by leukocytes recruited by CXCL9-11), we performed a survival study using C57BL/6 mice that received intraperitoneal (i.p.) injections of control serum or anti-CXCL9, anti-CXCL10, anti-CXCL11, anti-CXCL9+anti-CXCL10, anti-CXCL9+anti-CXCL10+anti-CXCL11, or anti-CXCR3 serum 24 hr prior to intranasal spore challenge and then daily throughout the experiment, using published protocols (see references 12, 13, 65, 108). The anti-CXCL9, CXCL10, and CXCL11 neutralizing antibodies have been validated in published work (see references 12, 19, 108). As shown in FIG. 9, mice that received anti-CXCL9, anti-CXCL9+anti-CXCL10, or anti-CXCL9+anti-CXCL10+anti-CXCL11 had significantly decreased survival after spore challenge. The other groups, including animals that received anti-CXCR3, had no significant difference in survival compared to normal serum controls that received spore challenge. These findings suggest that CXCL9, CXCL10, CXCL11 have significant direct antimicrobial effects against B. anthracis in vivo that may be independent of cell recruitment of CXCR3-expressing cells.

Determining that FtsX is a target of CXCL9, CXCL10, and CXCL11 using an in vivo model of infection.

Both wildtype B. anthracis and the ΔftsX chemokine-resistant mutant will be used in a mouse model of pulmonary infection to determine whether FtsX is a target for CXCL9, CXCL10, and CXCL11, leading to a protective antimicrobial effect in vivo. C57BL/6 mice are resistant to pulmonary infection with B. anthracis Sterne strain, but are susceptible to B. anthracis introduced by other routes of inoculation (e.g., subcutaneous). In contrast, A/J mice are highly susceptible to B. anthracis Sterne strain infection introduced via any of the above routes of inoculation. Thus, it would appear that C57BL/6 mice have an effective pulmonary host defense response/mechanism that is present or is generated in the lungs of mice infected with this pathogen. We previously found that lungs from C57BL/6 mice had significantly higher levels of CXCL9 and CXCL10 induced after intranasal inoculation of spores than did those from A/J mice. As noted above, we have also observed that neutralization of CXCL9, CXCL9/CXCL10, or CXCL9/CXCL10/CXCL11 rendered C57BL/7 mice susceptible to an inhalational disease (FIG. 9). Using the two mouse strains and a chemokine-resistant B. anthracis ΔftsX strain, we will further investigate the role of the interferon-inducible (ELR−) CXC chemokines during lung infection.

Without wishing to be bound by any particular theory, it is hypothesized herein that CXCL9, CXCL10, and CXCL11 have a direct antimicrobial effect both in vitro and in vivo against B. anthracis via FtsX such that absence of FtsX will render resistant mice susceptible to infection. We will determine whether the absence of FtsX causes normally resistant C57BL/6 mice to become susceptible to pulmonary infection. The study groups will be: 1) C57BL/6 mice+intranasal B. anthracis Sterne strain (parent strain) spores; 2) C57BL/6 mice+intranasal B. anthracis ΔftsX spores; and 3) C57BL/6 mice+intranasal B. anthracis Sterne strain (parent strain) spores+anti-CXCL9/CXCL10/CXCL11 neutralizing antibodies.

Mouse survival will be followed over a 20-day period following spore challenge. A minimum of 10 animals per group×3 groups=30 mice will be required for survival studies. We will assess burden of infection caused by the wildtype and the ΔftsX strain of B. anthracis by determining bacterial colony forming units (CFUs) and histopathology in the lungs as the initial site of infection and in the kidneys as a measure of bacterial dissemination to other organs. Using CFU data in conjunction with histopathology, we will determine whether there is more severe localized lung infection and/or if there is increased dissemination of bacteria to distal organs as a consequence of the absence of FtsX. The lungs and kidneys from animals will be harvested at an early and a later time point (e.g., day 2 and day 7 post-infection) for determination of bacterial CFUs from tissue samples (+/− heat treatment since spores are heat resistant whereas vegetative bacilli are heat sensitive) plated on BHI agar plates and incubated overnight at 37° C. In these studies, a minimum of three mice per group will be required per time point for these determinations (i.e., three mice per group per time point×3 groups×2 time points=18 mice). We will collect tissues (lungs, mediastinal lymph nodes, spleen, kidneys, liver) for histopathology to assess tissue damage, infiltration of leukocytes into the tissues, and spore/bacilli burden and localization/distribution within the tissues.

The samples will be reviewed and graded for the level of inflammation using the same severity scale as previously described (see references 12, 13, 108). The tissues will also be stained and examined for spores and bacilli, using published protocols (see reference 94). A minimum of 3 animals per group will be needed for histopathology=3 mice per group×3 groups×2 time points=18 mice. Thus, a total of 30+18+18=66 mice will be needed for these studies. For 3 replicate experiments, a total of 66×3=198 mice total will be required. Statistical analyses will be used to compare the data from each group to their respective control groups as well as between treatment groups.

Expected results and interpretations: Our data (FIG. 9) support the hypothesis that CXCL9, CXCL10, and CXCL11 are involved in the resistance of C57BL/6 mice to intrapulmonary B. anthracis infection such that neutralization of CXCL9, CXCL9/CXCL10, or CXCL9/CXCL10/CXCL11 renders C57BL/6 mice susceptible to pulmonary anthrax infection (FIG. 9). Furthermore, we have data showing that CXCL10−/− mice have increased spore/vegetative bacilli CFUs after spore challenge compared to that of the C57BL/6 parent strain. We expect that, in contrast to exhibiting resistance to wildtype B. anthracis, C57BL/6 mice inoculated with B. anthracis ΔftsX will succumb to infection with dissemination and mortality rates similar to or higher than the C57BL/6 mice inoculated with wildtype B. anthracis+anti-CXCL9/CXCL10/CXCL11 neutralizing serum.

Determine that interferons promote host defense against B. anthracis infection through the induction of CXCL9, CXCL10, and CXCL11.

Our data support that CXCL9, CXCL10, CXCL11, and IFN-γ are generated in the lungs as early as 1-6 hours after spore challenge; however, type 1 interferons were not measured in those experiments. We will determine which interferons are primarily responsible for inducing CXCL9, CXCL10, CXCL11 after spore challenge to determine how chemokines, as potential therapeutics, could be induced after a host has acquired the infection. Our working hypothesis is that type 1 and type 2 interferons are responsible for inducing these interferon-inducible (ELR−) CXC chemokines during anthrax infection.

Initially, we will perform intranasal spore challenges of IFN-y receptor knockout (IFN-γR KO) mice (Jackson Labs), using the C57BL/6 parent strain as a control. Lungs will be harvested at 1, 6, 24, 48 hrs post-infection (same time points as in ref. 26) for: a) CFU determinations and b) ELISAs to measure CXCL9, CXCL10, CXCL11 levels in lung homogenates. CFU determination will be performed using heated and unheated aliquots to assess spore CFUs (i.e., from heated samples) and the total number of spore+bacilli CFUs (i.e., from unheated samples). A minimum of three mice per group will be needed for tissue CFU determination and chemokine quantification at each of the four time points. Thus, a minimum of 3 mice per group×2 groups×4 time points=24 mice. After these fundamental data are obtained, mouse survival will be monitored over a 20-day period. A minimum of 10 animals per group×2 groups=20 mice for survival studies. Therefore, a total of 24+20=44 mice×3 replicate experiments=132 mice will be needed for these experiments. Statistical analyses will be used to compare data from each group to their respective control groups and between treatment groups.

We anticipate that the IFN-γR KO mice will exhibit markedly increased susceptibility to B. anthracis challenge. We predict that the levels of CXCL9, CXCL10, and CXCL11 in lung homogenates will be low compared to the C57BL/6 parent strain and that CFUs will be higher in the IFN-γR KO mice. These results would support our hypothesis that IFN-γ is key in inducing the interferon-inducible (ELR−) CXC chemokines during B. anthracis infection. If we find opposite results (i.e., that the IFN-γR KO mice remain resistant like the C57BL/6 parent strain), then it is likely that the type 1 interferons play a key role.

Develop a therapeutic strategy in a pre-clinical animal model with interferon induction of CXCL9, CXCL10, and CXCL11 to treat bacterial infections.

A pre-clinical animal model will be used to test the hypothesis that interferon-inducible (ELR−) CXC chemokines can function as therapeutics. Since CXCL9, CXCL10, and CXCL11 are potently induced by type 1 and type 2 interferons, we will focus on testing the utility of administering exogenous interferons as a therapeutic strategy for B. anthracis infection. A major advantage to the use of exogenous interferons is that type 1 interferons (IFN-α/β) and type 2 interferon (IFN-γ) are well-studied, FDA-approved drugs for human use, primarily for infectious diseases such as viral infections (type 1 interferons) and mycobacterial diseases (IFN-γ). Thus, there is a track record for clinical use of these immunomodulatory agents that we can draw upon for our proposed experiments. Especially pertinent to this proposal is the precedent in the literature that IFN-β or the Type 1 inducer (poly-ICLC) confers protection in mice infected with B. anthracis Ames strain (see reference 107).

In a pilot experiment, we injected A/J mice with recombinant murine IFN-γ (20,000 units i.p.), collected lungs at 0, 1, 6, 18, and 24 hrs for homogenization, and measured the levels of CXCL9, CXCL10, and CXCL11 in the homogenates by ELISA. We found that CXCL9, CXCL10, and CXCL11 levels peaked at 6 hours with levels of 6645±1399 pg/ml, 5503±1022 pg/ml, and 1631±356 pg/ml, respectively; these concentrations were within the range of the levels we previously observed in our studies of resistant C57BL/6 mice. Animals that were monitored for 72 hours (endpoint of the experiment) after IFN-γ administration remained healthy. Thus, we can induce CXCL9, CXCL10, and CXCL11 in the lungs using exogenous IFN-γ. We hypothesize that the results of administration of IFN-γ in a susceptible mouse strain will lead to the development of novel immunomodulatory approaches for post-exposure prophylaxis or treatment of anthrax.

We will test the effectiveness of administration of exogenous interferons, focusing initially on IFN-γ, as an immunomodulatory agent for treating B. anthracis pulmonary infection. Our pilot studies noted above with IFN-γ used an i.p. route of administration, and we will initially plan to administer via the i.p. route for survival studies since Walberg et al. found that i.p. administration of exogenous IFN-β provided greater protection that by the intranasal route (see reference 107). One caveat is that the same group found that administration of the type 1 inducer (poly-ICLC) via intranasal route was more protective than via the i.p. route, so the route of administration is an important variable that may require further testing. The arms of the study will be: 1) sham-infected mice+IFN-γ (as a control to ensure that IFN-γ is not contributing to morbidity/mortality of the mice); 2) spore-infected mice without IFN-γ (as a control to ensure that spore challenge worked); 3) spore-infected A/J mice +IFN-γ; and 4) spore-infected A/J mice+IFN-γ+anti-CXCL9/CXCL10/ CXCL11 neutralizing Abs (to test whether a protective effect conferred by IFN-γ is due to the production of CXCL9, CXCL10, and CXCL11). Measurements will include: 1) host survival (monitored for 10-15 days); 2) CXCL9, CXCL10, and CXCL11 levels in the lungs of animals at days 2, 5, and 10 after spore challenge; 3) Lung and kidney bacterial CFU determination to assess localized burden of infection as well as dissemination of organisms to other organs; 4) histopathology to assess tissue damage in the lungs. A minimum of 10 animals per group×4 groups=40 animals will be needed for survival studies. A minimum of 3 animals per group×4 groups will be needed for CFU determination, histopathology, and interferon-inducible (ELR−) CXC chemokine measurement by ELISA=12×4 outcome measurements=48 animals. Thus, a total of 40+48 mice=88 mice×3 replicates=264 mice will be needed.

With IFN-γ treatment, we anticipate that the A/J mice will have improved survival after spore challenge. In contrast, we anticipate that administration of IFN-γ plus neutralizing Abs against CXCL9/CXCL10/CXCL11 will result in the mice being highly susceptible to anthrax infection as seen with spore-challenged control A/J mice.

Walberg et al. (see reference 107) found that IFN-⊖ or the type 1 inducer poly ICLC conferred a protective effect for Swiss Webster mice infected with B. anthracis Ames strain. Mouse strain, choice of interferon, and dose/route of interferon administration are all potential variables. By measuring CXCL9, CXCL10, and CXCL11 levels generated in the lungs and assessing histopathology at various time points after spore challenge and while the animals are receiving IFN-γ will help us assess the appropriateness or potentially, the under- or over-responsiveness of the host response. It is also possible that type I interferons (e.g., IFN-α/β) or a combination of IFN-α/β and IFN-γ are the more relevant interferons (or interferon combinations) for inducing a protective effect in our pre-clinical model.

The in vitro and in vivo experimental approaches and translational nature of the proposed project will allow extensive characterization of a novel antimicrobial effect whereby CXCL9, CXCL10, and CXCL11 produced in the lungs have direct antimicrobial effects against B. anthracis spores and bacilli. We recently identified a putative bacterial target from a B. anthracis transposon mutagenesis library screen; the finding of FtsX as a target of a chemokine (directly or indirectly) is an entirely novel finding that opens up exciting avenues of investigation that should lead to innovative therapeutic strategies for treating and/or preventing pulmonary anthrax. These findings will likely extend beyond B. anthracis and have therapeutic impact on infections caused by a range of pathogenic and potentially, multi-drug resistant bacteria.

### EXAMPLE 5

Susceptibility of stationary phase organisms to interferon-inducible (ELR−) CXC chemokines

CXCL10 has been found to exert a markedly more potent effect against stationary phase B. anthracis Sterne strain 7702 (wildtype) organisms (see FIGS. 11A-B). Overnight cultures were either diluted back in fresh medium and grown to exponential phase prior to addition of buffer control or CXCL10 at 8 μg/ml (ie, ˜EC₅₀ value, see FIG. 11A) or used directly from overnight cultures by spinning down, reconstituting in same volume fresh medium plus buffer control or CXCL10 at 8 μg/ml (FIG. 11B). Aliquots were plated out for CFU determination after an incubation of 30 min or 1 hr. A concentration curve for CXCL10 against stationary phase organisms is shown in (FIG. 11C) with an EC₅₀ value determined to be 0.33+/−0.05 μg/ml. Each experiment was performed 3 separate times in triplicates. n.d., not detected.

The EC₅₀ value (0.33+/−0.05 μg/ml) for CXCL10 (FIG. 11C) is >10-fold more potent against stationary phase organisms compared to the EC₅₀ value determined for exponential phase organisms (as shown in FIG. 12B for the wildtype B. anthracis Sterne strain designated “7702 wt” in the graph). Importantly, the stationary phase organisms were placed in fresh culture medium at the time of the assay with CXCL10 so that, for the short assay incubation period of 30-60 minutes, there are nutrients present. Since the assay medium is not nutrient depleted, the finding that CXCL10 is more effective appears to not be simply due to a lack of nutrients for the organisms making them less fit or a lack of a nutrient or other component in the medium that could otherwise compete with CXCL10 for targeting FtsX or other target.

### EXAMPLE 6

Generation of a B. anthracis ΔftsX mutant strain.

Markerless allelic exchange was used to create a deletion mutant of the ftsX gene in wildtype B. anthracis Sterne strain (designated “ΔftsX”), using protocols of Dr. Stibitz (see references 30, 63, 76). Growth characteristics are shown in FIG. 12A for wildtype B. anthracis Sterne strain 7702 and ΔftsX. The ΔftsX strain grows more slowly than wildtype strain. The ΔftsX strain has a distinctive phenotype such that bacilli grow in “kinked” chains due to various angles produced at septations between individual bacilli. Sporulation occurs with ΔftsX but with a lower yield than that of the parent strain. We confirmed resistance of ΔftsX to CXCL10 (FIG. 12B). Furthermore, we found that ΔftsX was also resistant to CXCL9 and CXCL11, which supports that CXCL9, CXCL10, and CXCL11 have a common target in vegetative bacteria. Additionally, in contrast to B. anthracis Sterne 7702 strain, the ΔftsX exponential and stationary phase organisms are both resistant to CXCL10.

### EXAMPLE 7

Determining the Localization of CXCL10 in the Bacterial Cells Relative to FtsX

Co-localization studies. To assess localization of CXCL10 in the bacterial cells and whether it interacts directly with FtsX, we will take the following approach. Since there are no FtsX-specific antibodies available at this time, we will generate a tagged version of the B. anthracis FtsX. We plan to generate an FtsX-GFP fusion protein with the GFP located at the C-terminal end of FtsX using allelic exchange methodology previously employed in B. anthracis. Our choice of GFP is based on published studies of GFP fusion proteins in B. anthracis and successful expression and use of FtsX-GFP fusion proteins generated in E. coli, B. subtilis, and other bacterial species (see references 7, 31, 49, 97). Advantages to using a GFP tag are that we will be able to monitor the location and expression of FtsX at various stages of growth during the experiments, which may prove important if B. anthracis is killed by CXCL10 by, for example, disruption of cell division by inhibiting septal ring formation (FtsX localizes to septal rings in E. coli and B. subtilis).

Importantly, the tag introduced into FtsX must not interfere with the function of the transporter. Since the substrate transported by FtsX is unknown, we will test for potential disruption of FtsX function by assessing bacterial growth in medium alone (no chemokine), monitor kinetics of cell division in log phase, monitor formation of septal rings using a GFP tagged version of FtsX under fluorescence microscopy as per published protocols (see references 31, 49), and assess the ability of bacilli to form spores (since FtsX in B. subtilis is thought to play a critical role in sporulation). We will perform EM to assess the structural integrity of the strain expressing tagged FtsX versus wildtype strain at various stages of vegetative growth and sporulation.

Once a B. anthracis strain with a GFP-tagged FtsX is created and tested, we will examine the susceptibility of the organism to CXCL10 to ensure that addition of the GFP tag has not altered the antimicrobial effect of CXCL10 against the bacilli. We will perform co-localization studies with CXCL10 using immunofluorescence/ confocal microscopy to study the interaction of CXCL10 with FtsX at various time points. B. anthracis cells that produce FtsX-GFP will be fixed and permeabilized using standard protocols (see reference 60) and tested to ensure that the fixation process did not reduce or quench the GFP signal. If this does occur, an alternative approach would be to add anti-GFP antibodies after the bacteria are fixed and permeabilized followed by fluorescent-labeled secondary antibody. Commercially available anti-CXCL10 Abs will be used followed by a (red) fluorescent-labeled secondary antibody. Immunofluorescence/confocal microscopy will be performed to determine the individual locations of the CXCL10 and FtsX in the bacteria and determine if there is co-localization by appearance of a yellow signal (overlap of red and green signals).

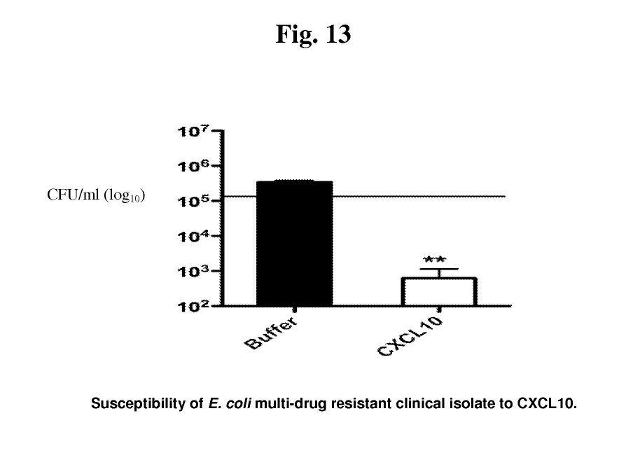

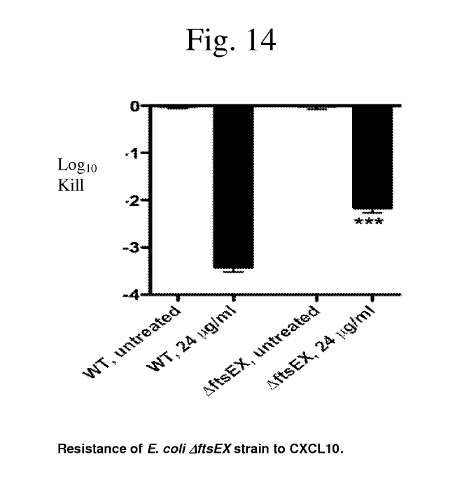

E. coli reagents. Much of the work on FtsX and its related ABC transporter components (FtsE, FtsY) has been performed in E. coli. An E. coli ΔftsEX mutant strain (see reference 7) and plasmids for complementation studies have been obtained from Dr. David Weiss (University of Iowa). Additionally, E. coli strains that express GFP-tagged FtsX or HA-tagged FtsE; plasmids for studying FtsY are also available. Importantly, it has been published by others that CXCL10 exhibits antimicrobial effects against lab strains of E. coli (see references 25, 112), and we have obtained data to support that E. coli multi-drug resistant clinical isolates are susceptible to CXCL10 antimicrobial activity (see FIG. 13). Furthermore, testing of E. coli ΔftsEX strain shows that this mutant strain exhibits increased resistance to CXCL10 (FIG. 14), supporting that FtsX (or FtsEX) is involved in susceptibility to CXCL10 in more than one bacterial species, namely in E. coli as well as B. anthracis. Complementation studies with plasmids encoding ftsX and/or ftsE are underway.

### EXAMPLE 8

Determining the Role of the Other ABC Transporter Components, FtsE and FtsY, in Susceptibility of Bacteria to CXCL10

Since ftsE is located immediately upstream of ftsX in the same operon, and both gene products (FtsE and FtsX) play a linked and pivotal role as the main components of the ABC transporter, we will investigate whether ftsE impacts the susceptibility of the organism to CXCL10. An important consideration is that FtsE is the ATP-binding component of the ABC transporter and as such, deletion of it may tell us whether CXCL 10 is actively transported by FtsX or not. Therefore, we will delineate whether CXCL10 (or a portion of it) is actively transported by FtsX or in some way requires an active functioning transporter. On the other hand, if deletion for ftsE has no impact on susceptibility to CXCL 10, then active transport seems an unlikely requirement and would indicate that CXCL10 requires FtsX for some other purpose in the cells. Although ftsY is located elsewhere in the B. anthracis genome, its gene product, FtsY, may play a role in the CXCL10 requirement for FtsX in order for it to exert its antimicrobial activity. For this reason, we will investigate this component of the ABC transporter as well.

The experimental approach will be similar to our approach for generating the B. anthracis ΔftsX mutant strain described above. In brief, we will use markerless allelic exchange to create a deletion mutant of the ftsE or ftsY gene in wildtype B. anthracis Sterne strain (designated ΔftsE or ΔftsY, respectively). Also, we will generate a double deletion mutant of ftsE and ftsX (i.e., ΔftsEX) in order to study the impact of the absence of these components of the ABC transporter. Complementation studies will be performed using plasmids with the genes for ftsE, ftsX, or ftsY, using plasmid constructs similar to those already used and validated in B. anthracis. Controls for complementation studies will include use of empty plasmid vectors as well as non-transformed wildtype (parent) bacterial strains.

For each B. anthracis mutant strain created, we will test growth characteristics and assess the viability of the organisms under various growth conditions. We will draw upon the E. coli and B. subtilis literature to assess particular growth requirements of the mutants such as salt and sucrose to help maintain viability. Additionally, these may be temperature sensitive mutants based on the literature, and temperature requirements may need to be carefully assessed; for example, the E. coli ΔftsEX mutant strain grows better at 30° C. and in the presence of salt and sucrose for osmotic stabilization. Susceptibility testing with CXCL10 will be performed using CFU determination or Alamar Blue assay. Modifications to the assay will be guided by the information gained from establishment of optimal growth conditions. To date, we have used tissue culture medium with physiological salt concentrations as well as other ions and proteins, so a requirement for salt and sucrose in the assay medium is not anticipated to affect the CXCL10 susceptibility assays that we routinely perform. We expect that, if CXCL10 requires the active transport function of FtsX, then the ΔftsE mutant strain should also be resistant to CXCL10-mediated killing as observed for ΔftsX. If, however, CXCL10 (or a portion of it) does not require the active transport by FtsX, then the ΔftsE mutant strain should remain susceptible to CXCL10. I

Fractionation and Co-Immunoprecipitation Studies.

To further study localization of CXCL10 in vegetative cells, we will perform fractionation studies using published protocols to separate bacterial cell wall, membrane, and cytosolic fractions at various time points using CXCL10-treated wildtype Sterne strain and a strain that expresses tagged FtsX. We will assess localization of FtsX and CXCL10 in the fractions using commercially available antibodies to the FtsX tag and to CXCL10. Controls will include use of wildtype Sterne strain vs. ΔftsX (the latter to compare the effect of the absence of FtsX on CXCL10 localization) plus the B. anthracis strain that expresses tagged FtsX. Gel electrophoresis followed by Western blot analysis will be used to determine which fraction, if any, contains CXCL10. Fraction purity will be determined by Western blot analysis in which the presence or absence of Protective Antigen (a cytosolic/secreted protein) or the S layer protein EA1 (which fractionates with the cell wall) is examined.

Co-immunoprecipitation studies using anti-CXCL10 antibodies will be performed to test CXCL10 interaction with FtsX and/or identify other interacting proteins. B. anthracis wildtype Sterne strain and ΔftsX will be lysed by sonication, and aliquots of whole lysates (or fractionated lysates) will be incubated with CXCL10 followed by immunoprecipitation of CXCL10 and its interacting proteins using commercially available anti-CXCL10 Abs and protein-G beads. Controls will include buffer controls and appropriate isotype Ab controls. Gel electrophoresis to separate proteins will be performed, followed by silver staining for protein visualization; candidate bacterial targets will be identified by mass spectrometry performed at our UVA Biomolecular Research Core Facility.

We anticipate that fractionation studies will reveal that CXCL10 localizes to the cell membrane fraction and that co-immunoprecipitation studies will reveal that CXCL10 interacts with FtsX. It is anticipated that other proteins may be immunoprecipitated with CXCL10, and those proteins deemed significant (i.e., not due to non-specific binding) will be identified by mass spectrometry.

Site-directed mutagenesis studies to determine key regions of FtsX required for CXCL 10 antimicrobial activity.

We hypothesize that CXCL10 interacts with the predicted extracellular portions of FtsX, designated Loops 1 & 2 in FIG. 6, with particular attention to Loop 1 based on the length of the loop, the number of negatively charged amino acids, and the region of sequence similarity to the CXCL10 receptor, CXCR3. Accordingly, we anticipate that the interaction between CXCL10 and FtsX involves a direct interaction with FtsX Loop 1 at a location where there is a net negative charge distribution (more specifically, in the region of amino acids 54-80 with similarity to the CXCR3 receptor binding region for CXCL10). We anticipate that co-localization experiments will reveal that the proteins interact at the cell membrane. We also predict that performing site-directed mutagenesis of extracellular portions will abrogate the interaction and the antimicrobial effect of CXCL10.

To assess which portions of FtsX interact with CXCL10, we will use allelic exchange to create deletion mutants of segments of Loop 1 and Loop 2. If deletion of a segment of FtsX abrogates the CXCL10 effect on the bacilli, we will narrow our studies to focus on key amino acids responsible for the interaction and/or effect of CXCL10. To do this, we will perform site-directed mutagenesis with substitution of neutral amino acids (alanine) for select amino acids in the portion of FtsX that may be responsible for the interaction or effect of CXCL10. We will initially target negatively charged amino acids that are clustered together since the net charge distribution is likely to play an important role in the interaction with CXCL10, which has a positively charged carboxyl terminus. The ability of the site-directed mutagenesis to disrupt interactions between the interferon-inducible (ELR−) CXC chemokine and FtsX will be assessed by in vitro susceptibility testing of the mutant bacterial strain to the interferon-inducible (ELR−) CXC chemokine Using a GFP-tagged version of FtsX, we will perform: 1) co-localization studies with immuno fluorescence microscopy; and 2) immunoprecipitation coupled with Western blot analyses to determine if the mutated FtsX can be co-precipitated with Abs to CXCL10.

### Identifying the Region(s) of CXCL10 Responsible for its Antimicrobial Effect

Identifying the region of CXCL10 responsible for its antimicrobial activity is an important aspect that could lead not only to development of a valuable tool for carrying out further mechanistic experiments but also potentially lead to a therapeutic reagent for testing as an antimicrobial agent. There are two very interesting and key considerations: 1) CXC10 has highly positively charged C-terminus that forms a predicted α-helix (FIG. 1) with similarity to defensins and other cationic antimicrobial peptides; and 2) Sequence alignment of B. anthracis FtsX and the known CXCL10 receptor, CXCR3, reveals that the two proteins share ˜45% amino acid sequence similarity in one region of the extracellular Loop 1 of each protein; the region in CXCR3 (amino acids 9-35) includes a key domain for binding the N-terminal region of CXCL10. We believe the C-terminal a-helical region of CXCL10 is responsible for its direct antimicrobial activity while the N-terminal portion of CXCL 10 may play a role in facilitating interaction with its target, FtsX. This could potentially be somewhat analogous to cholesterol-dependent cytolysins that bind to a cholesterol receptor and insert a different portion of the molecule into the eukaryotic membrane as an oligomer to form a pore, causing cell death.

Testing CXCL10 Effect on Membrane Integrity.

Two complementary, dye-based assays will be used to measure possible CXCL10-mediated increases in membrane permeability as compare to untreated and CC chemokine controls: propidium iodide (PI) uptake and diacetyl-fluorescein (DAF) release. PI uptake assays will be performed by including PI in the treatment sample wells. PI uptake by bacilli, which correlates to a loss of membrane integrity, will be monitored over a time course by fluorescence microscopy and/or direct measurement of sample well fluorescence. For DAF release assays, bacilli will be cultured in the presence of DAF resulting in uptake and subsequent hydrolysis to fluorescein, which is stored intracellularly. Supernatants from untreated and CXCL10-treated samples will be collected, and extracellular fluorescein released through membrane permeabilization will be measured. Heat-killed bacilli will be used as positive control for both the PI uptake and DAF release assays. Untreated bacilli will serve as negative control.

### Site-Directed Mutagenesis of CXCL10 or its Antimicrobial Peptide.

Working with CXCL10 or a peptide that retains its antimicrobial activity, we will investigate the effects of generating mutated forms of the protein/peptide in which one or more lysine residues in the region of interest (e.g., positively charged C-terminal region) has been substituted with a neutral amino acid, alanine Initially, substitutions of 1-3 lysines centrally located in the α-helix will be performed, assuming that the distribution of these amino acids in the α-helical turns leave them highly exposed such that they likely play a key role in charge distribution of the molecules. If the positively charged C-terminal region of CXCL10 is responsible for its antimicrobial activity, as predicted by the IL-8 literature, we anticipate that a C-terminal peptide will retain activity. However, if the N-terminal region of CXCL10 plays a role in the interaction of CXCL10 with FtsX or other target, then a C-terminal peptide alone may exhibit reduced or no antimicrobial activity.

### REFERENCES

1. Abbas A. K. et al., 1996. Nature 383:787-793.
2. Abramova F. A., L. M. Grinberg, O. V. Yampolskaya, D. H. Walker. 1993. Pathology of inhalational anthrax in 42 cases from the Sverdlovsk outbreak in 1979. Proc. Natl. Acad. Sci. USA 90:2291-2294.
3. Agrawal A., and B. Pulendran. 2004. Anthrax lethal toxin: A weapon of multisystem destruction. Cell. Mol. Life Sci. 61: 2859-2865.
4. Akhtar P., et al., J. Bacteriol. 191:2493-2500.
5. Alanis A. J. 2005. Resistance to antibiotics: Are we in the post-antibiotic era? Arch. Med. Res. 36:697-705.
6. Allen S., S. Crown, and T. Handel. 2007. Chemokine: receptor, structure, and antagonism. Annu Rev. Immunol. 25:787-820.
7. Aly S., T. Laskay, J. Mages, A. Malzan, R. Lang, and S. Ehlers. 2007. J. Pathol. 212:295-305
8. Baldari C. T., F. Tonello, S. R. Paccani, and C. Montecucco. 2006. Trends Immunol. 27:434-440.
9. Banks D. J., S. C. Ward, and K. A. Bradley. 2006. New insights into the functions of anthrax toxin. Exp. Rev. Mol. Med. 8:1-17.
10. Bayer, A. S., P. McNamara, M. R. Yeaman, N. Lucindo, T. Jones, A. L. Cheung, H. G. Sahl, and R. A. Proctor. 2006. J. Bacteriol. 188:211-222.
11. Beatty M. E., D. A. Ashford, P. M. Griffin, R. V. Tauxe, and J. Sobel. 2003. Gastrointestinal anthrax: review of the literature. Arch. Intern. Med. 163:2527-2531.
12. Belperio J. A. et al., 2002. J. Clin. Invest. 110:1703-1716. [PMCID: PMC151632]
13. Belperio J. A., M. P. Keane, M. D. Burdick, J. P. Lynch, III, Y. Y. Xue, K. Li, D. J. Ross, and R. M. Strieter. 2002. Critical role for CXCR3 chemokine biology in the pathogenesis of bronchiolitis obliterans syndrome. J Immunol. 169:1037-1049. [PMID: 12097412]
14. Boyden E. D. and W. F. Dietrich. 2006. Nalp1b controls mouse macrophage susceptibility to anthrax lethal toxin. Nat. Genetics 38:240-244.
15. Bozue J., K. L. Moody, C. K. Cote, B. G. Stiles, A. M. Friedlander, S. L. Welkos, and M. L. Hale. 2007. Bacillus anthracis spores of the bc1A mutant exhibit increased adherence to epithelial cells, fibroblasts, and endothelial cells but not to macrophages. Infect. Immun. 75:4498-4505. [PMCID: PMC1951178]
16. Braibant M., et al. 2000. The ATP binding cassette (ABC) transport systems of Mycobacterium tuberculosis. FEMS Microbiol. Rev. 24:449-467.
17. Brittingham K. C., G. Ruthel, R. G. Panchal, C. L. Fuller, W. J. Ribot, T. A. Hoover, H. A. Young, A. O. Anderson, and S. Bavari. 2005. Dendritic cells endocytose Bacillus anthracis spores: Implications for anthrax pathogenesis. J. Immunol. 174:5545-5552.
18. Bunai K. et al., 2004. Electrophoresis 25:141-155.
19. Burdick M. D., L. A. Murray, M. P. Keane, Y. Y. Xue, D. A. Zisman, J. A. Belperio, and R. M. Strieter. 2005. CXCL11 attenuates bleomycin-induced pulmonary fibrosis via inhibition of vascular remodeling. Am. J. Resp. Crit. Care Med. 171:261-268. [PMID: 15502109]
20. Cadieux N., C. Bradbeer, E. Reeger-Schneider, W. Koster, A. K. Mohanty, M. C. Wiener, and R. J. Kadner. 2002. Identification of the periplasmic cobalamin-binding protein BtuF of Escherichia coli. J. Bactiol. 184:706-717.
21. Chakravarty S.D., J. Xu, B. Lu, C. Gerard, J. Flynn, and J. Chan. 2007. The chemokine receptor CXCR3 attenuates the control of chronic Mycobacterium tuberculosis infection in BALB/c mice. J. Immunol. 178:1723-1735.
22. Chhibber S., S. Kaur, and S. Kumari. 2008. Therapeutic potential of bacteriophage in treating Klebsiella pneumoniae B5055-mediated lobar pneumonia in mice. J. Med. Microbiol. 57:1508-1513.
23. Cleret A., A. Quesnel-Hellman, A. Vallon-Eberhard, B. Verrier, S. Jung, D. Viddal, J. Mathieu, and J. N. Tournier. 2007. Lung dendritic cells rapidly mediate anthrax spore entry through the pulmonary route. J. Immunol. 178:7994-8001.
24. Cole A. M., T. Ganz, A. M. Liese, M. D. Burdick, L. Liu, and R. M. Strieter. 2001. Cutting edge: IFN-inducible ELR− CXC chemokines display defensin-like antimicrobial activity. J. Immunol. 167:623-627. PMID: 11441062 [PubMed - indexed for MEDLINE]
25. Cole A. M., P. Dewan, and T. Ganz. 1999. Innate antimicrobial activity of nasal secretions. Infect. Immun. 67:3267-3275. [PMCID: PMC116505]
26. Crawford M. A., Y. H. Zhu, C. S. Green, M. D. Burdick, P. Sanz, F. Alem, A. D. O'Brien, B. Mehrad, R. M. Strieter, and M. A. Hughes. 2009. Antimicrobial effects of interferon-inducible CXC chemokines against Bacillus anthracis spores and bacilli. Infect. Immun. 77:1664-1678. [PMCID: PMC2663164]
27. Cybulski Jr., R. J., P. Sanz, F. Alem, S. Stibitz, R. L. Bull, and A. D. O'Brien. 2009. Four superoxide dismutases contribute to Bacillus anthracis virulence and provide spores with redundant protection from oxidative stress. Infect. Immun. 77:274-285. [PMCID: PMC2612278]
28. De Leeuw E., B. Graham, G J. Phillips, C. M. ten Hagen-Jongman, B. Oudega, and J. Luirink 1999. Molecualr characterization of Eschericheria coli FtsE and FtsX. Mol. Microbiol. 31:983-993.
29. De Smet K., and R. Contreras. 2005. Human antimicrobial peptides: defensins, cathelicidins, and histatins. Biotech. Lett. 27:1337-1347.
30. Dixon T. C., A. A. Fadl, T. M. Koehler, J. A. Swanson, and P. C. Hanna. 2000. Early Bacillus anthracis macrophage interactions: intracellular survival survival and escape. Cell. Microbiol. 2:453-63.
31. Dixon T. C., M. Meselson, J. Guillemin, and P. C. Hanna. 1999. Anthrax. N. Engl. J. Med. 341:815-826.
32. Doolan D. L., et al. 2007. The US Capitol bioterrorism anthrax exposures: Clinical epidemiological and immunological characteristics. J. Inf. Dis. 195:174-184.
33. Dun M., and A. Peschel. 2002. Chemokines meet defensins: The merging concepts of chemoattractants and antimicrobial peptides in host defense. Infect. Immun. 70:65616-6517. [PMCID: PMC133099]
34. Egesten A., M. Eliasson, H. M. Johansson, A. I. Olin, M. Morgelin, A. Mueller, J. E. Pease, I. M. Frick, and L. Bjorck. 2007. The CXC chemokine CXCL9 is important in innate immunity against Streptococcus pyogenes. J. Infect. Dis. 195:684-693.
35. Eswarappa S. M., K. K. Panguluri, M. Hensel, and D. Chakravortty. 2008. The yejABEF operon of Salmonella confers resistance to antimicrobial peptides and contributes to its virulence. Microbiology-Sgm 154:666-678.
36. Falagas M. E. and I. A. Bliziotis. 2007. Pandrug resistant Gram-negative bacteria: the dawn of the post-antibiotic era? Intl. J. Antimicrob. Agents. 29:630-636.
37. Fensterl V., and G. C. Sen. 2009. Interferons and viral infections. Biofactors 35:14-20.
38. Gank K. D., M. R. Yeaman, S. Kojima, N. Y. Yount, H. Park, J. E. Edwards, S. G. Filler, and Y. Fu. 2008. SSD1 is integral to host defense peptide resistance in Candida albicans. Eukaryotic Cell 7:1318-1327.
39. Ganz T. 2003. Defensins: Antimicrobial peptides of innate immunity. Nat. Rev. Immunol. 3 :710-720.
40. Ganz T., and R. I. Lehrer. 1995. Defensins. Pharmacol. Ther. 66:191-205.
41. Garti-Levi S., R. Hazan, J. Kain, M. Fujita, S. Ben-Yehuda. 2008. The FtsEX ABC transporter directs cellular differentiation in Bacillus subtilis. Mol. Microbiol. 69:1018-1028
42. Giorno R., J. Bozue, C. Cote, T. Wenzel, K. S. Moody, M. Mallozzi, M. Ryan, R. Wang, R. Zielke, J. R. Maddock, A. Friedlander, S. Welkos, and A. Driks. 2007. Morphogenesis of the Bacillus anthracis spore. J. Bacteriol. 189: 691-705.
43. Giorno R., M. Mallozi, J. Bozue, K. S. Moody, A. Slack, D. Qiu, R. Wang, A. Friedlander, S. Welkos, and A. Driks. 2009. Localization and assembly of proteins comprising the outer structures of the Bacillus anthracis spore. Microbiol. SGM 155:1133-1145.
44. Glomski I. J., F. Dumetz, G. Jouvion, M. R. Huerre, M. Mock, and P. L. Goossens. 2008. Inhaled noncapsulated Bacillus anthracis in A/J/mice: nasopharynx and alveolar space as dual portals of entry, delayed dissemination, and specific organ targeting. Microbes Infect. 10:1398-1404. [PMID: 18762267]
45. Glomski I. J., J. P. Corre, M. Mock, and P. L. Goossens. 2007. Noncapsulated toxinogenic Bacillus anthracis presents a specific growth and dissemination pattern in naïve and protective antigen-immune mice. Infect. Immun. 75:4754-4761.
46. Gold J. A., Y. Hoshino, S. Hoshino, M. B. Jones, A. Nolan, and M. D. Widen. 2004. Exogenous gamma and alpha/beta interferon rescues human macrophages from cell death induced by Bacillus anthracis. Infect. Immun. 72:1291-1297. [PMCID: PMC356021]
47. Gold J. A., Y. Hoshino, M. B. Jones, S. Hoshino, A. Nolan, and M. D. Weiden. 2007. Exogenous interferon-alpha and interferon-gamma increase lethality of murine inhalational anthrax. PLoS One 2:e736.
48. Goossens P. L. 2009. Animal models of human anthrax: The quest for the holy grail. Mol. Aspects Med. 30:467-480.
49. Guidi-Rontani C. 2002. The alveolar macrophage: the Trojan horse of Bacillus anthracis. Trends Microbiol. 10:405-409.
50. Guidi-Rontani C., M. Levy, H. Ohayon, and M. Mock. 2001. Fate of germinated Bacillus anthracis spores in primary murine macrophages. Mol. Microbiol. 42:931-938.
51. Guidi-Rontani C., M. Weber-Levy, E. Labruyere, and M. Mock. 1999. Germination of Bacillus anthracis spores within alveolar macrophages. Mol. Microbiol. 31:9-17.
52. Hancock W. W., B. Lu, W. Gao, V. Csizmadia, K. Faia, J. A. King, S. T. Smiley, M. Ling, N. P. Gerard, and C. Gerard. 2000. Requirement of the chemokine receptor CXCR3 for acute allograft rejection. J. Exp. Med. 192:1515-1520. [PMCID: PMC2193193]
53. Hardison J. L., et al., 2006. Infect. Immun. 74:125-134.
54. Harvill E.T., G. Lee, V. K. Grippe, and T. J. Merkel. 2005. Complement depletion renders C57BL/6 mice sensitive to the Bacillus anthracis Sterne strain. Infect. Immun. 73:4420-4422.
55. Hokeness K. L. et al. 2007. CXCR3-dependent recruitment of antigen-specific T lymphocytes to the liver during murine cytomegalovirus infection. J. Viol. 81:1241-1250.
56. Holty J-E. C., D. M. Bravata, H. Liu, R. A. Olshen, K. M. McDonald, and D. K. Owens. 2006. Systematic review: A century of inhalational anthrax cases from 1900 to 2005. Ann. Int. Med. 144:270-280.
57. Hsieh M. F., S. L. Lai, J. P. Chen, J. M. Sung, Y. L. Lin, B. A. Wu-Hsieh, C. Gerard, A. Luster, F. Liao. 2006. Both CXCR3 and CXCL10/IFN-inducible protein 10 are required for resistance to primary infection by dengue virus. J. Immunol. 177:1855-1863.
58. Hughes, M. A., C. W. Lee, C. F. Holm, S. Ghosh, L. A. Lockhart, S. L. Reed, and B. J. Mann. 2003.
   Identification of Entamoeba histolytica thiol-specific antioxidant as a GalNAc lectin-associated protein. Mol. Biochem. Parasitol. 127:113-120.
59. Hvorup R. N., B. A. Goetz, M. Niederer, K. Hollenstein, E. Perozo, and K. P. Locher. 2007. Asymmetry in the structure of the ABC trasnporter-binding protein complex BtuCD-BtuF. Science 317:1387-1390.
60. James J. S. 2004. AIDS Treat News 7.
61. Janes B. K., and S. Stibitz. 2006. Infect. Immun. 74:1949-1953.
62. Jernigan J. A., et al., 2001, Emerg. Inf., Dis. 7:933-944.
63. Jernigan D. B., et al., and the National Anthrax Epidemiologic Investigation Team. 2002. Investigation of bioterrorism-related anthrax, United States, 2001: Epidemiologic findings. Emerg. Inf. Dis. 8:1019-1028.
64. Joshi S. G., H. B. Cymet, G. Kerkvliet, and T. Cymet. Anthrax in America 2001-2003. J. Natl. Med. Assoc. 96:344-350.
65. Keane M. P., Belperio J. A., Y. Y. Xue., M. D. Burdick, and R. M. Strieter. 2004. Depletion of CXCR2 inhibits tumor growth and angiogenesis in a murine model of lung cancer. J. Immuol. 172:2853-2860. PMID: 14978086
66. Koprivnjak T., C. Weidenmaier, A. Peschel, and J. P. Weiss. 2008. Wall teichoic acid deficiency in Staphylococcus aureus confers selective resistance to mammalian group IIA phospholipase A2 and human b-defensin 3. Infect. Immun. 76:2169-2176.
67. Kovacs, M. et al., 2006. J. Bacteriol. 188:5797-5805.
68. Lande R., et al., 2003. J. Immunol. 170:1174-1182.
69. Liu L., M. K. Callahan, D. Huang, and R. M. Ransohoff. 2005. Chemokine receptor CXCR3: an unexpected enigma. Curr. Top. Dev. Biol. 68:149-181.
70. Liu M. T., D. Armstrong, T. A. Hamilton, and T. E. Lane. 2001. Expression of MIG (monokine induced by interferon-gamma) is important in T lymphocyte recruitment and host defense following viral infection of the central nervous system. J. Immunol. 166:1790-1795.
71. Loetscher M., B. Gerber, P. Loetscher, S. A. Jones, L. Piali, I. Clark-Lewis, M. Baggiolini, and B. Moser. 1996. Chemokine receptor specific for IP10 and MIG: structure, function, and expression in activated T lymphocytes. J. Exp. Med. 184:963-969. [PMCID: PMC2192763]
72. Loetscher M., P. Loetscher, N. Brass, R. Meese, and B. Moser. 1998. Lymphocyte-specific chemokine receptor CXCR3: regulation, chemokine binding and gene localization. Eur. J. Immunol. 28:3696-3705.
73. Loving C. L., M. Kennett, G. M. Lee, V. K. Grippe, and T. J. Merkel. 2007. Murine aerosol challenge model of anthrax. Infect. Immun. 75:2689-2698.
74. Loving C. L. et al., 2009. Infect. Immun. 77:255-265.
75. Luster A. D., R. Alon, and U. H. von Andrian. 2005. Immune cell migration in inflammation: present and future therapeutic targets. Nat. Immunol. 6:1182-1190.
76. Luster A. D. 2002. The role of chemokines in linking innate and adaptive immunity. Curr. Opin. Immunol. 14:129-135.
77. Luster A. and J. Ravetch. 1987. Biochemical characterization of a gamma interferon-inducible cytokine (IP-10). J. Exp. Med. 166:1084-1097.
78. Luster A. D. 1998. Chemokines—chemotactic cytokines that mediate inflammation. N. Engl. J. Med. 33 8:436-445.
79. Lyons C. R., J. Lovchik, J. Hutt, M. F. Lipscomb, E. Wang, S. Heninger, L. Berliba, and K. Garrison. 2004. Murine model of pulmonary anthrax: kinetics of dissemination, histopathology, and mouse strain susceptibility. Infect. Immun. 72: 4801-4809. [PMCID: PMC470666]
80. MacKenzie F. M., M. J. Struelens, K. J. Towner, and I. M. Gould. 2005. Report of the Consensus Conference on Antibiotic Resistance; Prevention and Control (ARPAC). Clin. Microbiol. Infect. 11:938-954.
81. Mehrad B., M. P. Keane, and R. M. Strieter. 2007. Chemokines as mediators of angiogenesis. Thromb. Haemost. 97:755-762. PMID: 17479186 [PubMed—indexed for MEDLINE]
82. Moberly B. J., F. Shafa, and P. Gerhardt. 1966. Structural details of anthrax spores during stages of transformation into vegetative cells. J. Bacteriol. 92:220-228.
83. Mock M., and A. Fouet. 2001. Anthrax. Annu Rev. Microbiol. 55:647-671.
84. Moser B., and P. Loetscher. 2001. Lymphocyte traffic control by chemokines Nat. Immunol. 2:123-128.
85. Oliva C. R., M. K. Swiecki, C. E. Griguer, M. W. Lisanby, D. C. Bullard, C. L. Turnbough Jr., and J. F. Kearney. 2008. The integrin Mac-1 (CR3) mediates internalization and directs Bacillus anthracis spores into professional phagocytes. Proc. Natl. Acad. Sci. USA 105:1261-1266.
86. Oppenheim J. J., A. Biragyn, L. W. Kwak, and D. Yang. 2003. Roles of antimicrobial peptides such as defensins in innate and adaptive immunity. Ann. Rheum. Dis. 62:17-21.
87. Popham D. L. 2002. Specialized peptidoglycan of the bacterial spore: the inner wall of the lockbox. Cell Mol. Life Sci. 59:426-433.
88. Puliti M. C. von Hunolstein, F. Bistoni, P. Mosci, G. Orefici, and L. Tissi. 2000. Influence of interferon-γ administration on the severity of experimental group B streptococcal arthritis. Arthritis Rheum. 43:2678-2686.
89. Qin S., J. B. Rottman, P. Myers, N. Kassam, M. Weinblatt, M. Loetscher, A. E. Koch, B. Moser, and C. R. Mackay. 1998. The chemokine receptors CXCR3 and CCR5 mark subsets of T cells associated with certain inflammatory reactions. J. Clin. Invest. 101:746-754.
90. Rabin R. L., M. K. Park, F. Liao, R. Swofford, D. Stephany, and J. M. Farber. 1999. Chemokine receptor responses on T cells are achieved through regulation of both receptor expression and signaling. J. Immunol. 162:3840-3850.
91. Ross J. M. 1957. The pathogenesis of anthrax following the administration of spores by the respiratory route. J. Pathol. Bacteriol. 73: 485-494.
92. Rossi D. and A. Zlotnik. 2000. The biology of chemokines and their receptors. Annu Rev. Immunol. 18:217-242.
93. Russell B. H., R. Vasan, D. R. Keene, and Y. Xu. 2007. Bacillus anthracis internalization by human fibroblasts and epithelial cells. Cell. Microbiol. 9:1262-1274.
94. Sanz P., L. D. Teel, F. Alem, H. M. Carvalho, S. C. Darnell, and A. D. O'Brien. 2008. Detection of Bacillus anthracis spore germination in vivo by bioluminescence imaging. Infect. Immun. 76:1036-1047.
95. Sauty A., M. Dziejman, R. A. Taha, A. S. Iarossi, K. Neote, E.A. Garcia-Zepeda, Q. Hamid, and A. D. Luster. 1999. The T cell-specific CXC chemokines IP-10, MIG, and I-TAC are expressed by activated human bronchial epthithelial cells. J. Immunol. 162:3549-3558.
96. Seiler P., P. Aichele, S. Bandermann, A. E. Hauser, B. Lu, N. P. Gerard, C. Gerard, S. Ehlers, H. J. Mollenkopf, and S. H. Kaufmann. 2003. Early granuloma formation after aerosol Mycobacterium tuberculosis infection is regulated by neutrophils via CXCR3-signaling chemokines Eur. J. Immunol. 33:2676-2686.
97. Shahabuddin S., R. Ji, P. Wang, E. Brailoiu, N. Dun, Y. Yang, M. O. Aksoy, and S. G. Kelsen. 2006. CXCR3 chemokine receptor-induced chemotaxis in human airway epithelial cells: role of p38 MAPK and PI3K signaling pathways. Am. J. Physiol. Cell Physiol. 291:C34-9.
98. Shibaya M., M. Kubomichi, and T. Watanabe. 1991. The genetic basis of host resistance to Bacillus anthracis in inbred mice. Vet. Microbiol. 26:309-312.
99. Spurrell J. C., S. Wiehler, R. S. Zaheer, S. P. Sanders, and D. Proud. 2005. Human airway epithelial cells produce IP-10 (CXCL10) in vitro and in vivo upon rhinovirus infection. Am. J. Physiol. 289:L85-95.
100. Svensson M., S. Zubairi, A. Maroof, F. Kazi, M. Taniguchi, and P. M. Kaye. 2005. Invariant NKT cells are essential for the regulation of hepatic CXCL10 gene expression during Leishmania donovani infection. Infect. Immun. 73:7541-7547.
101. Takamatsu H., and K. Watabe. 2002. Assembly and genetics of spore protective structures. Cell. Mol. Life Sci. 59:434-444.
102. Tsai W. C., R. M. Strieter, J. M. Wilkowski, K. A. Bucknell, M. D. Burdick, S. A. Lira, and T. J. Standiford. 1998. Lung specific transgenic expression of KC enhances resistance to Klebsiella pneumoniae in mice. J. Immunol. 16:2435-2440.
103. Tsai W. C., R. M. Strieter, D. A. Zisman, J. M. Wilkowski, K. A. Bucknell, G.-H. Chen, and T. J. Standiford. 1997. Nitric oxide is required for effective innate immunity against Klebsiella pneumoniae. Infect. Immun. 65:1870-1875. [PMCID: PMC175233]
104. Turk B. E. 2007. Manipulation of host signalling pathways by anthrax toxins. Biochem. J. 402:405-417.
105. Vasquez R. E., and L. Soong. 2006. CXCL10/gamma interferon-inducible protein 10-mediated protection against Leishmania amazonensis infection in mice. Infect. Immun. 74:6769-6777. [PMCID: PMC1698098]
106. Villar C. C., and A. Dongari-Bagtzoglou. 2006. Therapeutic modulation of cytokines in chronic infectious diseases. Curr. Pharm. Design 12:2329-2348.
107. Walberg K., S. Baron, J. Poast, B. Schwartz, L. Izotova, S. Pestka, and W. Peterson. 2008. Interferon protects mice against inhalation anthrax. J. Interferon Cytokine Res. 28:597-601.
108. Wallace K. L., M. A. Marshall, S. I. Ramos, J. A. Lannigan, J. J. Field, R. M. Strieter, and J. Linden. 2009. NKT cells mediate pulmonary inflammation and dysfunction in murine sickle cell disease through production of IFN-{gamma} and CXCR3 chemokines Blood 114:667-676. [PMCID: PMC2713467]
109. Weidenmaier C., A. Peschel, V. A. J. Kempf, N. Lucindo, M. R. Yeaman, and A. S. Bayer. 2005. DltABCD- and MprF-mediated cell envelope modifications of Staphylococcus aureus confer resistance to platelet microbicidal proteins and contribute to virulence in a rabbit endocarditis model. Infect. Immun. 73:8033-8038.
110. Welkos S. L., T. J. Keener, and P. H. Gibbs. 1986. Differences in susceptibility of inbred mice to Bacillus anthracis. Infect. Immun. 51:795-800.
111. Xanthou G., T. J. Williams, J. E. Pease. 2003. Molecular characterization of the chemokine receptor CXCR3: evidence for the involvement of distinct extracellular domains in a multi-step model of ligand binding and receptor activation. Eur. J. Immunol. 33:2927-2936.
112. Yang D., Q. Chen, D. M. Hoover, P. Staley, K. D. Tucker, J. Lubkowski, and J. Oppenheim. 2004. Many chemokines including CCL20/MIP-3a display antimicrobial activity. J. Leuk. Biol. 74:448-455.
113. Yeaman M. R., and N. Y. Yount. 2007. Unifying themes in host defense effector polypeptides. Nat. Rev. Microbiol. 5:727-740.
114. Yount N. Y., A. S. Bayer, Y. Q. Xiong, and M. R. Yeaman. 2006. Advances in antimicrobial peptide immunobio logy. Biopolymers 84:435-458.
115. Zaman M. S., A. Goyal, G. P. Dubey, P. K. Gupta, H. Chadra, T. K. Das, M. Ganguli, and Y. Singh. 2005. Imaging and analysis of Bacillus anthracis spore germination. Microscopy Res. Techniq. 66:307-311.
116. Zemansky J., B.C. Kline, J.J. Woodward, J.H. Leber, H. Marquis, and D.A. Portnoy. 2009. Development of a mariner-based transposon and identification of Listeria monocytogenes determinants, including the peptidyl-prolyl isomerase PrsA2, that contribute to its hemolytic phenotype. J. Bacteriol. 191:3950-3964.
117. Zeng X., et al., 2005. Infect. Immun. 73:8226-8236.
118. Zisman D. A. et al., 1998. Alcohol Clin. Exp. Res. 22:621-627.
119. Zlotnik A., and 0. Yoshie. 2000. Chemokines: a new classification system and their role in immunity. Immunity 12:121-127.

1. A method of neutralizing spores of a prokaryotic pathogenic organism, said method comprising
contacting said pathogenic organisms with a composition comprising an interferon-inducible (ELR−) CXC chemokine.

2. The method of claim 1 wherein the interferon-inducible (ELR−) CXC chemokine comprises
a peptide sequence selected from the group consisting of SEQ ID NO: 3, SEQ ID NO: 6 and SEQ ID NO: 9, or a peptidomimetic derivative thereof.

3. The method of claim 1 wherein the spores are from an organism selected from the group consisting of Bacillus anthracis, Bacillus cereus, Clostridium difficile, Clostridium botulinum, Clostridium perfringens, Clostridium tetani and Clostridium sordellii.

4. The method of claim 3 wherein the spores are Bacillus anthracis or Clostridium difficile spores.

5. The method of claim 2 wherein the peptide, or peptidomimetic derivative thereof comprises i) SEQ ID NO: 1, SEQ ID NO: 2, SEQ ID NO: 4, SEQ ID NO: 5, SEQ ID NO: 7 or SEQ ID NO: 8, ii) a peptide fragment of SEQ ID NO: 1, SEQ ID NO: 2, SEQ ID NO: 4 SEQ ID NO: 5, SEQ ID NO: 7 and SEQ ID NO: 8, or a peptide having at least 90% amino acid sequence identity with i) or ii).

6. The method of claim 5 wherein the peptide, or peptidomimetic derivative thereof comprises SEQ ID NO: 1, SEQ ID NO: 2, SEQ ID NO: 4, SEQ ID NO: 5 or a peptide having at least 95% amino acid sequence identity with SEQ ID NO: 1, SEQ ID NO: 2, SEQ ID NO: 4 or SEQ ID NO: 5.

7. The method of claim 2 wherein the peptide, or peptidomimetic derivative thereof, comprises a sequence selected from the group consisting of SEQ ID NO: 15 and SEQ ID NO: 16.

8. The method of claim 5 wherein the peptide, or peptidomimetic derivative thereof, comprises a sequence selected from the group consisting of SEQ ID NO: 1, SEQ ID NO: 2, SEQ ID NO: 4 and SEQ ID NO: 7.

9. (canceled)

10. (cancled)

11. The method of claim 5 wherein said composition further comprises a lipid vesicle and said peptide or peptidomimetic derivative is encapsulated within the lipid vesicle, or linked to the surface of said lipid vesicle.

12. The method of claim 11 wherein said composition further comprises a supplemental anti-microbial agent.

13. The method of claim 12 wherein the supplemental anti-microbial agent is an antibiotic.

14. An antimicrobial composition, said composition comprising
a non-native peptide, said peptide comprising a sequence selected from i) SEQ ID NO: 3, SEQ ID NO: 6 or SEQ ID NO: 9, or ii) a peptide having at least 90% amino acid sequence identity with SEQ ID NO: 3, SEQ ID NO: 6 or SEQ ID NO: 9, or peptidomimetic derivative of i) or ii), with the proviso that said peptide does not consist of SEQ ID NO: 1, SEQ ID NO: 2, SEQ ID NO: 3, SEQ ID NO: 4, SEQ ID NO: 5, SEQ ID NO: 7 or SEQ ID NO: 8.

15. The antimicrobial composition of claim 14 wherein said peptide comprises a sequence selected from i) SEQ ID NO: 3 or SEQ ID NO: 6 or a peptide having at least 95% amino acid sequence identity with SEQ ID NO: 3 or SEQ ID NO: 6.

16. The antimicrobial composition of claim 14 wherein said peptide comprises a sequence of SEQ ID NO: 15 or SEQ ID NO: 16.

17. (canceled)

18. (canceled)

19. The antimicrobial composition of claim 14 wherein said composition further comprises a lipid vesicle and said peptide or peptidomimetic derivative is encapsulated within the lipid vesicle, or linked to the surface of said lipid vesicle.

20. The antimicrobial composition of claim 19 wherein said composition further comprises a supplemental anti-microbial agent.

21. (canceled)

22. A pharmaceutical composition comprising
the non-native peptide of claim 14; and
a pharmaceutically acceptable carrier.

23. (canceled)

24. The method of claim 6 wherein said spores of the prokaryotic pathogenic organism have colonized a host organism and have entered into a stationary growth phase.

25-34. (canceled)

35. An antigenic composition comprising an isolated peptide comprising the sequence of SEQ ID NO: 10 or an contiguous 8 amino acid fragment of SEQ ID NO: 10.

36. The antigenic composition of claim 35 further comprising an adjuvant.

37-50. (canceled)
